(19) United States
(12) Patent Application Publication
(10) Pub. No.: US 2025/0102493 A1
(43) Pub. Date: Mar. 27, 2025

(54) COMPOSITIONS AND METHODS FOR TREATMENT OF ANEUPLOID CANCERS

(71) Applicants: Yale University, New Haven, CT (US); Cold Spring Harbor Laboratory, Cold Spring Harbor, NY (US)

(72) Inventors: Jason Sheltzer, New Haven, CT (US); Asad Aziz Lakhani, Huntington Station, NY (US); Christine Scaduto, New York, NY (US); Vishruth Girish, Baltimore, MD (US); Sarah Thompson, Hamden, CT (US); Leanne Brown, Shamong, NJ (US)

(21) Appl. No.: 18/893,726

(22) Filed: Sep. 23, 2024

Related U.S. Application Data
(60) Provisional application No. 63/584,783, filed on Sep. 22, 2023.

(51) Int. Cl. G01N 33/50; C12N 5/071; C12N 5/09; C12N 9/22; C12N 15/11; C12N 15/90

(52) U.S. Cl. CPC G01N 33/5011; C12N 5/0629; C12N 5/0631; C12N 5/0679; C12N 5/0682; C12N 5/0693; C12N 9/22; C12N 15/111; C12N 15/90; C12N 2310/20; C12N 2510/00

(57) ABSTRACT

Systems and methods for the selective ablation of a target chromosome or fragment thereof in a cell have been established. Methods for the production of isogenic diploid-like cells, derived from aneuploid precursor cells have been developed. The isogenic diploid-like cells are genetically equivalent to the precursor cells, except for the loss of a target chromosome or fragment thereof. The systems provide isogenic “aneuploidy-loss” cancer cells. The cells can be used to, e.g., screen aneuploidy-selective therapies. In some forms, the methods target aneuploid cancers harboring extra copies of Chr1q, which encodes the UCK2 gene, by contacting the cells with agents activated by UCK2. Exemplary agents include RX-3117 and 3-deazauridine. Methods targeting aneuploid cancers harboring extra copies of Chr7p, which encodes the AHR gene, by contacting the cells with agents that bind to AHR, such as CGS-15943.

### CROSS-REFERENCE TO RELATED APPLICATIONS

This application claims the benefit of and priority to U.S. Provisional Application No. 63/584,783 filed Sep. 22, 2023, and which is incorporated herein by reference in its entirety.

### STATEMENT REGARDING FEDERALLY SPONSORED RESEARCH

This invention was made with government support under CA276666 awarded by National Institutes of Health. The government has certain rights in the invention.

### REFERENCE TO SEQUENCE LISTING

The Sequence Listing XML submitted as a file named “YU_8572_US.xml,” created on Sep. 23, 2024, and having a size of 43,668 bytes is hereby incorporated by reference pursuant to 37 C.F.R. § 1.834 (c) (1).

### FIELD OF THE INVENTION

The invention is generally related to the field of cancer, and compositions and methods of characterization and treatment thereof.

### BACKGROUND OF THE INVENTION

Chromosome copy number changes, otherwise known as aneuploidy, are a ubiquitous feature of tumor genomes (Taylor, et al., Cancer Cell. 33, 676-689.e3 (2018), Gordon, et al., Nat. Rev. Genet. 13, 189-203 (2012)). Nearly 90% of cancers exhibit aneuploidy, and it affects a greater percentage of the cancer genome than any other somatic genetic alteration (Taylor, et al., Cancer Cell. 33, 676-689.c3 (2018)). Whereas the most frequent focal copy number alterations were observed in just 14% of cancer specimens (Beroukhim, et al., Nature. 463, 899-905 (2010)), an analysis of over 10,500 patient datasets from The Cancer Genome Atlas (TCGA) revealed the most frequent chromosomal arm alterations were present in 30% of tumors, and a typical tumor exhibits a median of 3 gains and 5 losses of chromosomal arms (Ben David & Amon, Nat Rev Genet, 21(1):44-62. doi: 10.1038/s41576-019-0171-x. Epub 2019 Sep. 23). Thus, tumors must select for favorable aneuploid karyotypes, and chromosomal gain or loss must provide a fitness advantage beyond that provided by focal amplifications or deletions (Davoli, et al., Cell. 155, 948-962 (2013)). Not only can different cancer types be identified by their aneuploidy profiles (Auslander et al., Nat Commun., 11(1):1234. doi: 10.1038/s41467-020-15094-2 (2020)), but focal copy number alterations also harbor significantly greater prognostic power than mutational profiles (Smith & Sheltzer, eLife. 7, e39217 (2018)).

While the pervasiveness of aneuploidy in cancer has been known for over a century (Boveri, J. Cell Sci. 121, 1-84 (2008), Hardy & Zacharias, Cell Biol. Int. 29, 983-992 (2005)), the role of aneuploidy in tumor development has remained controversial (Vasudevan, et al., Nat. Rev. Cancer. 21, 89-103 (2021), Sheltzer & Amon, Trends Genet. TIG. 27, 446-453 (2011), Sheltzer, et al., Cancer Cell. 31, 240-255 (2017), Weaver, et al., Cancer Cell. 11, 25-36 (2007)). Chromosome gains have been proposed to serve as a mechanism for increasing the dosage of tumor-promoting genes that are found within altered regions (Davoli, et al., Cell. 155, 948-962 (2013), Sack, et al., Cell. 173, 499-514.e23 (2018)). However, proof of this hypothesis is lacking, and it has alternately been proposed that aneuploidy could arise as a result of the loss of checkpoint control that frequently occurs in advanced malignances (Zimonjic, et al., Cancer Res. 61, 8838-8844 (2001)). Indeed, individuals with Down syndrome, which is caused by the triplication of chromosome 21, have a markedly decreased risk of developing most solid cancers, indicating that in certain cases aneuploidy may actually have tumor-suppressive properties (Hasle, et al., Genet. Med. 18, 1151-1157 (2016)).

The ability to directly interrogate the role of aneuploidy in cancer has historically been limited by the experimental difficulties involved in manipulating entire chromosome arms. For decades, cancer researchers have used the standard tools of molecular genetics, including gene overexpression, knockdown, and mutagenesis, to develop a deep understanding of many individual oncogenes and tumor suppressors (Bister, Proc. Natl. Acad. Sci. U.S.A 112, 15259-15260 (2015), O'Loughlin, Annu. Rev. Cancer Biol. 3, 345-363 (2019)). For instance, the biological functions of genes like KRAS and TP53 were elucidated in part by creating and analyzing isogenic cell lines that express or lack these genes (Bunz, et al., Science. 282, 1497-1501 (1998), Shirasawa, et al., Science. 260, 85-88 (1993)). However, existing approaches for single-gene manipulations are insufficient to interrogate the chromosome-scale changes that affect hundreds of genes simultaneously. The consequences of eliminating specific aneuploid chromosomes from human cancer cells have not previously been established.

Studies of individual cancer driver genes led to the discovery of a phenomenon called “oncogene addiction”, in which loss or inhibition of a single oncogene is sufficient to induce cancer regression (Weinstein, Science. 297, 63-64 (2002)). For example, mutations in KRAS cause the development of pancreas cancer, and genetically ablating KRAS in a “KRAS-addicted” pancreas tumor blocks growth and triggers apoptosis (Waters, Cold Spring Harb. Perspect. Med. 8 (2018)). Previous cancer genome sequencing projects have revealed that the aneuploidy patterns observed in human tumors are non-random, and specific events like the gain of chromosome 1q and 8q occur more often than expected by chance (Taylor, et al., Cancer Cell. 33, 676-689.e3 (2018), Zack, et al., Nat. Genet. 45, 1134-1140 (2013)).

The systematic characterization of the pan-cancer consequences of aneuploidy, however, remains challenging for a variety of reasons. The simultaneous alteration in the dosage of hundreds of genes on the aneuploid chromosome means that differentiating between driver and passenger genes is difficult, and there is no established method for targeting aneuploid cancers.

Moreover, there is no currently established method to target aneuploidy as a therapeutic strategy.

Therefore, there is an urgent need for methods to characterize the pan-cancer consequences of aneuploidy.

Therefore, it is an object of the invention to provide methods to target aneuploidy as a therapeutic strategy.

It is another object of the invention to provide systems for differentiating between driver and passenger genes for characterization of the pan-cancer consequences aneuploidy.

It is another object of the invention to provide systems for identifying drugs targeting dosage-sensitive genes located on aneuploid chromosomes.

It is another object of the invention to provide systems for identifying drugs that elicit an additive or more than additive effect in targeting dosage-sensitive genes located on aneuploid chromosomes.

### SUMMARY OF THE INVENTION

Systems and methods for the selective ablation of a target chromosome or fragment thereof in a cell have been established. Methods for the production of isogenic diploid-like cells, derived from aneuploid precursor cells have been developed. The isogenic diploid-like cells are genetically equivalent to the precursor cells, except for the loss of a target chromosome or fragment thereof. A platform for the screening of active agents that are selectively toxic to aneuploid cells is also described, and drug discovery platforms that can yield drug targets and synergistic drug combinations for aneuploidy-stratified patient populations are provided.

Methods for creating a viable isogenic cell or tissue having an ablated chromosome or a fragment thereof from a precursor cell or tissue have also been developed. Typically, the methods include (i) introducing into a precursor cell or tissue a gene editing composition configured to cleave a target chromosome or target chromosome arm and optionally a nucleic acid configured to integrate into the target chromosome or target chromosome arm to form an isogenic cell or tissue including a cleaved target chromosome or cleaved target chromosome arm; and (ii) optionally isolating the isogenic cell. In some forms, the cleaved target chromosome or target chromosome arm is lost from the cell or tissue. Useful gene editing compositions are selected from Clustered Regularly Interspaced Short Palindromic Repeats/CRISPR associated protein (CRISPR/Cas), zinc finger nucleases (ZFN), and transcription activator-like effector nucleases (TALEN). Typically, the gene editing composition is a CRISPR/Cas composition, such as CRISPR/Cas composition having a guide RNA (gRNA) configured to cleave nucleotides proximal to a centromere in a chromosome or a chromosome arm, such as an aneuploid chromosome or aneuploid chromosome arm. In some forms, the CRISPR/Cas composition including a nucleic acid configured to integrate into an aneuploid chromosome arm also includes a reporter gene. Typically, the CRISPR/Cas composition is configured to cleave the aneuploid chromosome arm between the integrated reporter gene and a centromere of the aneuploid chromosome arm. Therefore, in some forms, the step of isolating the isogenic cell includes selecting a cell or tissue lacking the reporter gene. Exemplary reporter genes are selected from herpesvirus thymidine kinase (HSV-TK), Cytosine Deaminase, Diphtheria Toxin, and a fluorescent protein. In some forms, the reporter gene is herpesvirus thymidine kinase (HSV-TK) and the step of isolating includes contacting the cell or tissue with ganciclovir, and isolating an isogenic cell or tissue that is not susceptible to ganciclovir. In other forms, the gene editing composition includes a CRISPR/Cas composition, having a nucleic acid configured to integrate into a chromosome or a chromosome arm, such as an aneuploid chromosome or aneuploid chromosome arm includes a telomere repeat sequence. Therefore, in some forms, the CRISPR/Cas composition is configured to cleave nucleotides proximal to a centromere in the in a chromosome or a chromosome arm, such as an aneuploid chromosome or aneuploid chromosome arm, and integrate the telomere repeat sequence to provide a de novo telomere; and the step of isolating the isogenic cells or tissue includes selecting cells having the de novo telomere. An exemplary telomere repeat sequence includes ˜ 100 repeats of the human telomere seed sequence. In some forms, a telomere repeat sequence also includes a reporter gene, and the step of isolating the isogenic cell includes selecting a cell or tissue expressing the reporter gene. Typically, the cell is a human cell or tissue. Exemplary target chromosome include any one or more of chromosome 1, 2, 3, 4, 5, 6, 7, 8, 9, 10, 11, 12, 13, 14, 15, 16, 17, 18, 19, 20, 21, 22, X, or Y. Exemplary chromosome arms include any one or more selected from 1q, 1p, 2q, 2p, 3q, 3p, 4q, 4p, 5q, 5p, 6q, 6p, 7p, 7q, 8p, 8q, 9p, 9q, 10p, 10q, 11p, 11q, 12p, 12q, 13p, 13q, 14q, 14p, 15q, 15p, 16p, 16q, 17p, 17q, 18p, 18q, 19q, 19q, 20q, 20p, or 21q, 21p, 22q, 22p, Xp, Xq, Yp, or Yq. In certain forms, the target chromosome arm is selected from chromosome 1q, chromosome 7p, and chromosome 8q, for example, chromosome 1q. In some forms, the target chromosome or target chromosome arm expresses one or more genes selected from CYP2J2, MFSD2A, UCK1, UCK2, SLC16A1, UGT1A10, DCK, SLC26A2, SLC29A1, ACTB, ABCB1, SLC39A4, ADK, FADS2, SLC35F2, PDE3A, ABCC1, SULT1A1, NQO1, SLC47A1, SLFN11, SLFN12, DCAF15, SLC6A16, SLC19A1, and AHR. In some forms, the precursor cell or tissue is a cancer cell or cancer tissue, such as an aneuploid cancer cell or tissue. In some forms, the cancer cell or tissue is derived from a subject with cancer, such as an aneuploid cancer. Therefore, in some forms, the methods further include one or more steps of obtaining a cancer cell or tissue from a subject. Exemplary cancers are selected from f anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer, uterine cancer, acute myeloid leukemia, and adrenocortical carcinoma.

Isogenic cells, isogenic tissue and isogenic organoids formed from a precursor cell or tissue according to the described methods are also provided.

Methods for screening of aneuploid cancer cells or tissue for susceptibility to an active agent are also described. Typically, the methods include (a) contacting a viable aneuploid cancer cell or tissue with one or more active agents under conditions suitable for cell or tissue culture, whereby the aneuploid cell or tissue includes an additional chromosome or chromosome arm relative to an isogenic control cell or tissue, whereby the additional chromosome or chromosome arm includes one or more expressed genes, optionally whereby the one or more genes are differently expressed in the aneuploid cancer cell or tissue relative to the control cell or tissue; and (b) selecting the one or more active agents as selectively toxic to the aneuploid cancer cell or tissue when the agent reduces the viability of the aneuploid cancer cell or tissue to a greater extent than the reduction in viability of the isogenic control cell or tissue contacted by the same active agent(s) under the same conditions. In some forms, an isogenic control cell or tissue that lacks the additional chromosome or arm is derived from the aneuploid cancer cell or tissue by selective inactivation of the aneuploid chromosome or arm. In some forms, an isogenic control cell or tissue is derived from a precursor aneuploid cancer cell or tissue according to the described methods for producing isogenic cells. In some forms, the active agent(s) inhibit, reduce or otherwise interfere with the one or more differently expressed genes and/or expressed gene products. Exemplary differently expressed genes and/or expressed gene products include one or more importer pumps and/or enzymes, such as importer pumps and/or enzymes that are active on the active agent. In other forms, the enzyme modifies the structure of the active agent from a first form to a second form, for example, whereby the second form is more toxic to the aneuploid cell relative to the first form. Exemplary active agent(s) include one or more selected from austocystin D, tunicamycin, 3-bromopyruvate, Curcumin, Gemcitabine, decitabine, bis (maltolato) oxovanadium (IV), Gemcitabine, atorvastatin, simvastatin, pitavastatin, lovastatin, fluvastatin, tepoxalin, AICAR, 6-azacytidine and 5-methylcytidine, Triciribine, ML239, YM155, DCEBIO, zardaverine, Dasatinib, Oncrasin-1, HSP90 inhibitors, Mitoxantrone, disulfiram, DCEBIO, zardaverine, Indisulam, Temsirolimus, RX-3117, 3-deazauridine, Methotrexate, and CGS-15943. In some forms, the aneuploid cancer cells or tissue are autologous cells or tissue derived from a subject having cancer. An exemplary cancer is selected from anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer and uterine cancer, acute myeloid leukemia, and adrenocortical carcinoma. In some forms, the methods include one or more steps of treating the subject for cancer by administering the one or more active agents to the subject in an amount effective to reduce or prevent one or more symptoms of the cancer when the active agent(s) is selectively toxic to the aneuploid cancer cell. In some forms, the methods include, prior to step (a), one or more steps to identify the subject having cancer. For example, in some forms, the methods include, prior to step (a), one or more steps to identify the cancer as an aneuploid cancer.

Methods of treating an aneuploid cancer in a subject in need thereof, including administering to the subject a therapeutic agent in an amount effective to reduce or prevent one or more symptoms of the aneuploid cancer in the subject, where the therapeutic agent reduces the viability of aneuploid cancer cells more than the viability of isogenic control cells, are also described. Typically, the therapeutic agent is administered to the subject in an amount effective to reduce the viability of aneuploid cancer cells in the subject. In some forms, the aneuploid cancer cells aberrantly expresses one or more genes associated with an aneuploid chromosome or arm, and the therapeutic agent is active against the one or more aberrantly-expressed genes, or the expression products thereof. An exemplary cancer is selected from anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer and uterine cancer, acute myeloid leukemia, and adrenocortical carcinoma.

Methods for creating a viable isogenic cell or tissue or organoid having an ablated aneuploid chromosome or a fragment thereof from a precursor aneuploid cell or tissue, are provided. Typically, the methods include the steps of (i) introducing into an aneuploid precursor cell or tissue a gene editing composition configured to cleave an aneuploid chromosome arm and optionally a nucleic acid configured to integrate into the aneuploid chromosome arm to form isogenic cell or tissue including a cleaved aneuploid chromosome arm; and (ii) optionally isolating the isogenic cell or tissue. In some forms, the cleaved aneuploid chromosome arm is lost from the cell or tissue or organoid. Typically, the gene editing composition is a CRISPR/Cas composition, where the nucleic acid configured to integrate into the aneuploid chromosome arm includes a reporter gene, where the CRISPR/Cas composition is configured to cleave the aneuploid chromosome arm between the integrated reporter gene and a centromere of the aneuploid chromosome arm; and whereby isolating the isogenic cell or tissue or organoid includes selecting a cell or tissue or organoid lacking the reporter gene. In an exemplary form, the reporter gene is herpesvirus thymidine kinase (HSV-TK); and the isolating in step (ii) includes contacting the cell or tissue or organoid with ganciclovir, and isolating an isogenic cell or tissue or organoid that is not susceptible to ganciclovir. In other forms, the gene editing composition includes a CRISPR/Cas composition, the nucleic acid configured to integrate into the aneuploid chromosome arm includes a telomere repeat sequence, the CRISPR/Cas composition is configured to cleave nucleotides proximal to a centromere in the aneuploid chromosome arm and integrate the telomere repeat sequence to provide a de novo telomere; and isolating the isogenic cell or tissue or organoid includes selecting a cell or tissue or organoid including the de novo telomere. In some forms, the telomere repeat sequence includes ˜ 100 repeats of the human telomere seed sequence, and optionally a reporter gene, for example, such that isolating the isogenic cell or tissue or organoid includes selecting a cell expressing the reporter gene.

An exemplary precursor cell or tissue is or is part of a tissue graft, a prosthesis or an organoid. Isogenic cell(s) or tissue(s), implant(s), tissue grafts, prostheses or organoids formed from or including a precursor cell or tissue produced by any of the described methods are also provided.

The experiments below exemplify use of the platforms to identify chromosome 1q aneuploidy an effective marker for sensitivity to UCK2-targeting chemotherapeutic compounds. UCK2-dependent chemotherapies and methods of use thereof to treat cancer subjects are provided. Also provided is 1q aneuploidy as a biomarker for sensitivity to UCK2-targeting compounds, and methods of use thereof to stratify patient populations by aneuploidy status and improve therapeutic benefit.

### BRIEF DESCRIPTION OF THE DRAWINGS

The accompanying drawings, which are incorporated in and constitute a part of this specification, illustrate several embodiments of the disclosed method and compositions and together with the description, explain the principles of the disclosed method and compositions.

FIGS. 1A-1C show that specific chromosome gains arise early in tumor development and are mutually exclusive with driver gene mutations. FIG. 1A shows graphs of recurrent early gains of chromosome 1q in BRCA (n=38 tumor samples) and MEL (n=37 tumor samples). For each tumor type, the timing of gains was converted into ranks for genomic bins within a patient and computed the rank sums across patients for each bin. The normalized rank sums for each genomic bin are shown for BRCA and MEL. The large negative values indicate recurrent early initiating gains. normalized rank sums were used to test against the null hypothesis (no regions show recurrent early gain across patients). Bins from chromosome 1q reject this null for both tumor types (with 90% confidence level). FIG. 1B shows a heat map representation of a pan-cancer analysis of mutual exclusivity between mutations in 25 commonly-mutated cancer genes and chromosome arm gain events. FIG. 1C is a graph showing mutual exclusivity and co-occurrence patterns between one representative chromosome gain (+13q, bars at the top), and point mutations in several different cancer driver genes.

FIGS. 2A-2D show phenotypic effects of losing chromosome 1q-aneuploidy. FIG. 2A is a schematic showing chromosomal engineering strategies for the targeted deletion of chromosome arms: (1) REDACT-NS: using CRISPR-Cas9 homology-directed repair, a positive-negative selection cassette encoding a fluorescent reporter, a positive selection marker, and a negative selection marker (HSV thymidine kinase) were integrated at a centromere-proximal region on chromosome 1q. Arm loss was induced by generating a dsDNA break centromere-proximal to the cassette with Cas9, and isolated clonal populations of cells that were ganciclovir-resistant. (2) REDACT-TR: arm loss was induced by generating a dsDNA break at a centromere-proximal location with Cas9 while providing cells with an ectopic telomere seed sequence for repair. (3) ReDACT-CO: arm loss was induced by generating a dsDNA break at a centromere-proximal location with Cas9, and isolated clonal populations of cells. For all three approaches, clonal populations of cells were screened for targeted chromosome loss through TaqMan CNV assays and validated their karyotypes through SMASH sequencing. FIG. 2B shows graphs of 1q-disomic clones that exhibit decreased anchorage-independent growth for1q-disomic clones vs 1q-trisomic clones. FIG. 2C shows graphs of 1q-disomic clones that exhibit impaired xenograft growth in vivo. 1q-trisomic and 1q-disomic cells were injected contralaterally and subcutaneously into immunocompromised mice. The graphs display the mean±SEM for each trial. FIG. 2D shows graphs of 1q-disomic MCF10A clones transduced with HRASG12V that exhibit decreased anchorage-independent growth relative to 1q-trisomic MCF10A cells. FIG. 2E shows graphs of 1q disomic MCF10A clones transduced with HRASG12V clones exhibit impaired xenograft growth in vivo. 1q-trisomic and 1q-disomic cells were injected contralaterally and subcutaneously into immunocompromised mice. The graphs display the mean±SEM for each trial.

FIGS. 3A-3D show variable degrees of addiction to aneuploidy of chromosome 1q, 7p, and 8q. FIG. 3A shows a graph of 7p-disomic and 8q-disomic clones in A2058 that exhibit a milder deficit in anchorage-independent growth as compared to 1q-disomic clones. Boxes represent the 25th, 50th, and 75th percentiles of colonies per field, while the whiskers represent the 10th and 90th percentiles. Unpaired t-test, n=15 fields of view, data from representative trial (n≥2 total trials). ***p<0.0005. FIG. 3B shows graphs of 7p-disomic and 8q-disomic clones in A2058 that exhibit a moderate defect in xenograft growth. Wild-type (7p-trisomic and 8q-trisomic) cells and either 7p-disomic or 8q-disomic cells were injected contralaterally and subcutaneously into immunocompromised mice. The graphs display the mean±SEM for each trial. FIG. 3C shows graphs and related images of 8q-disomic clones in HCT116 that exhibit decreased anchorage-independent growth, and micrographs that display representative images of colony formation for the indicated cell lines. ***p<0.0005. FIG. 3D shows graphs of 8q-disomic clones in HCT116 that exhibit variable xenograft growth. 8q-trisomic and 8q-disomic cells were injected contralaterally and subcutaneously into immunocompromised mice. The graphs display the mean±SEM for each trial.

FIGS. 4A-4G show cancers rapidly recover chromosome 1q aneuploidy. FIG. 4A shows graphs of A2058 1q-disomic cells frequently evolve to recover a third copy of chromosome 1q during xenograft growth, with Tumor volume (0-2500 mm³) over time (0-25 Days) shown for each of A2058 ( [symbol] ) and 7p-loss c21 ( [symbol] ), respectively. Cell lines were rederived from tumors at the endpoint of the xenograft assays, and chromosome copy number was determined through TaqMan copy number assays. Mean±SEM, n=3 probes on targeted chromosome, data from representative trials are shown (n=2 total trials). The corresponding xenograft assays are shown on the left. FIG. 4B shows a graph of 1q-disomic clones that have evolved to regain 1q trisomy following xenograft growth and exhibit increased anchorage-independent growth relative to the pre-xenograft 1q-disomic parental cells. Boxes represent the 25th, 50th, and 75th percentiles of colonies per field, while the whiskers represent the 10th and 90^{th} percentiles. Unpaired t-test, n=15 fields of view, data from representative trial (n >2 total trials). FIG. 4C shows graphs of variable evolution of 7p-disomic cells that recover a third copy of chromosome 7p during xenograft growth, with Tumor volume (0-2000 mm³) over time (0-25 Days) shown for each of A2058 ( [symbol] ) and 7p-loss c1 ( [symbol] ), respectively. Mean±SEM, n=3 probes on targeted chromosome, data from representative trials are shown (n=2 total trials). The corresponding xenograft assays are shown on the left. FIG. 4D shows graphs of variable evolution of 8q-disomic cells that recover a third copy of chromosome 8q during xenograft growth. Mean±SEM, n=3 probes on targeted chromosome, data from representative trials are shown (n=2 total trials). The corresponding xenograft assays are shown on the left. FIG. 4E shows a graph of a regain of trisomy 1q occurs more frequently than regain of trisomy 7p or trisomy 8q. Tumors were classified as exhibiting regain if the mean copy number of the targeted chromosome was ≥ 2.5, as determined through TaqMan copy number assays. n=213 tumors, chi-squared test. FIG. 4F shows graphs of HCT116 8q-disomic clones evolved to gain a copy of chromosome 12 during xenograft assays, resulting in the acquisition of an extra copy of the KRASG13D allele. Cell lines were rederived from tumors harvested at the endpoint of xenograft assays and subjected to SMASH karyotyping and Sanger sequencing of KRAS. The xenograft growth curve is shown on the left, and representative SMASH karyotype profiles and Sanger sequencing chromatograms pre- and post-xenograft are shown on the right. Chromosome 8q and chromosome 12 are highlighted. FIG. 4G shows a graph of 8q-disomic clones that have evolved to acquire trisomy of chromosome 12 following xenograft growth exhibit increased anchorage-independent growth relative to the pre-xenograft 8q-disomic parental cells.

FIGS. 5A-5M show a single extra copy of MDM4 suppresses p53 signaling and contributes to the 1q trisomy addiction. FIG. 5A shows a graphical GSEA analysis of A2780 RNA-seq data. The graph reveals upregulation of the p53 pathway in the 1qdisomic clones, relative to the parental trisomy. FIG. 5B shows a heatmap displaying the upregulation of 10 p53 target genes in A2780 1q-disomic clones. The TK+ clone indicates a clone that harbors the CRISPR-mediated integration of the HSV-TK transgene but that was not treated to induce chromosome 1q-loss. FIG. 5C shows an image of Western blot analysis demonstrating activation of p53 signaling in 1q-disomic clones. GAPDH was analyzed as a loading control. The TK+ clone indicates a clone that harbors the CRISPR mediated integration of the HSV-TK transgene but that was not treated to induce chromosome 1q-loss. FIG. 5D shows a waterfall plot highlighting the most-significant instances of mutual exclusivity between chromosome arm gains and mutations in cancer-associated genes. FIG. 5E shows a boxplot displaying the TP53-mutation phenocopy signature (Fito-Lopez, et al., Prevalence, causes and impact of TP53-loss phenocopying events in human tumors (2022)) in cancers from TCGA, split based on whether the cancers harbor a non-synonymous mutation in TP53. FIG. 5F shows a scatterplot comparing the association between chromosome arm gains and the TP53-mutation phenocopy signature (Fito-Lopez, et al., Prevalence, causes and impact of TP53-loss phenocopying events in human tumors (2022)) in TP53-wildtype cancers from TCGA. Cancers with chromosome 1q gains are indicated. FIG. 5G shows a boxplot displaying the TP53-mutation phenocopy signature (Fito-Lopez, et al., Prevalence, causes and impact of TP53-loss phenocopying events in human tumors (2022)) in cancers from TCGA, split based on whether tumors harbor a gain of chromosome 1q. Only TP53-wildtype cancers are included in this analysis. FIG. 5H shows boxplots displaying the expression of three p53 target genes—CDKN1A (p21), RRM2B, and GADD45A—in cancers from TCGA split based on the copy number of chromosome 1q. Only TP53-wildtype cancers are included in this analysis. Boxplots represent the 25th, 50th, and 75th percentiles of the indicated data, while the whiskers represent the 10th and 90th percentiles of the indicated data. FIG. 5I shows a graph of a CRISPRi competition assay that demonstrates that gRNAs targeting MDM4 drop out over time in A2780 cells. In contrast, gRNAs targeting AAVS1 and PIP5K1A, another gene encoded on chromosome 1q, exhibit minimal depletion. FIG. 5J shows a schematic displaying the strategy for using paired CRISPR gRNAs to delete a single copy of MDM4 in a cell line with a trisomy of chromosome 1q. FIG. 5K shows a heatmap of 1q-disomic clones and MDM4+/+/KO clones in A2780 that exhibit comparable upregulation of p53 transcriptional targets, as determined through TaqMan gene expression assays. FIG. 5L shows a graph and accompanying images of MDM4+/+/KO clones that exhibit decreased anchorage-independent growth relative to the MDM4+/+/+ parental cell line. FIG. 5M shows graphs and accompanying images showing that induction of MDM4 cDNA in 1q-disomic clones in A2780 increases anchorage-independent growth. For the soft agar experiments in the data are from n=15 fields of view, and a representative trial is shown (n ≥2 total trials). Scale bars=250 μm. ***p<0.0005.

FIGS. 6A-6F show that gaining chromosome 1q increases sensitivity to UCK2 substrates. FIG. 6A shows a schematic of the metabolism of two pyrimidine analogs, RX-3117 and 3-deazauridine. UCK2, a kinase encoded on chromosome 1q, phosphorylates these compounds to produce cytotoxic derivatives that can poison DNA and RNA synthesis. FIG. 6B shows boxplots displaying the expression of UCK2 from the cancer cell line encyclopedia (left) and TCGA (right), divided based on the copy number of chromosome 1q. The boxplots represent the 25th, 50th, and 75th percentiles of the indicated data, while the whiskers represent the 10th and 90th percentiles of the indicated data. Data were analyzed using unpaired t-tests; n=10,331 samples from TCGA and 942 samples from CCLE. FIG. 6C shows a graph of expression of UCK2 protein in cancer cell lines with 1q trisomies or following aneuploidy elimination. FIG. 6D is a series of bar graphs showing cellular sensitivity of A2780 and MCF10A treated with different concentrations of RX-3117 or 3-deazauridine. Mean±SEM, data from representative trials are shown (n≥3 total trials). FIG. 6E is a schematic displaying cellular competition between trisomic and disomic cells. Under normal conditions, certain trisomies enhance cellular fitness, allowing these cells to overtake the population and enhance malignant growth (top). However, treatment with an “anti-trisomy” compound could selectively impair the growth of the aneuploid cells, keeping the population in a low-malignant state (bottom). FIG. 6F is a graph of cellular competition between fluorescently-labeled MCF10A 1q-trisomic and unlabeled 1q-disomic cells. These cells were mixed at a ratio of 20% to 80% and then cultured in either DMSO or 3-deazauridine. While the trisomic cells quickly dominate the population in drug-free medium, treatment with 3-deazauridine prevents the outgrowth of the 1q-trisomic subpopulation. Data from representative trial are shown (n=2 total trials). *p<0.05, **p<0.005, ***p<0.0005.

FIGS. 7A-7E show the identification of CGS-15943 as a compound that exhibits increased activity against cancer cells that are trisomic for chromosome 7p. FIG. 7A is a waterfall plot displaying all negative correlations between the expression of genes on chromosome 7p and drug sensitivity in the PRISM dataset (Corsello, et al., Nat. Cancer. 1, 235-248 (2020)). The AHR-CGS15493 dyad is indicated. FIG. 7B is a scatterplot displaying the correlation between AHR expression and CGS-15943 sensitivity. A linear regression line is plotted against the data. FIG. 7C is a schematic displaying how AHR expression is required for CGS-15943 sensitivity (O'Donnell, et al., Apoptosis Int. J. Program. Cell Death. 26, 307-322 (2021)). CGS-15943 binds to AHR and Arnt and causes this complex to upregulate the expression of proapoptotic genes. FIG. 7D is a series of graphs displaying the relationship between chromosome 7p copy number and AHR expression. (Left and center): Boxplots displaying the expression of AHR from the cancer cell line encyclopedia (left) and TCGA (center), divided based on the copy number of chromosome 7p. (Right): A bar graph displaying the expression of AHR in wild-type A2058 cells and A2058 7pdisomic cells. The boxplots represent the 25th, 50th, and 75th percentiles of the indicated data, while the whiskers represent the 10th and 90th percentiles of the indicated data. The bar graph represents the mean±SEM. Data were analyzed using unpaired t-tests. FIG. 7E is a graph showing cellular sensitivity of A2058 and A2058 7p-loss treated with different concentrations of CGS-15943. *p<0.05, **p<0.005, ***p<0.0005.

### DETAILED DESCRIPTION OF THE INVENTION

The disclosed method and compositions can be understood more readily by reference to the following detailed description of embodiments and the Examples included therein and to the Figures and their previous and following description.

### I. Definitions

“Introduce” in the context of genome modification refers to bringing in to contact. For example, to introduce a gene editing composition to a cell is to provide contact between the cell and the composition. The term encompasses penetration of the contacted composition to the interior of the cell by any suitable means, e.g., via transfection, electroporation, transduction, gene gun, nanoparticle delivery, etc.

“Homologous” refers to the sequence similarity or sequence identity between two polypeptides or between two nucleic acid molecules. When a position in both of two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or are homologous, then the two sequences are 60% homologous. By way of example, the DNA sequences ATTGCC and TATGGC share 50% homology. Generally, a comparison is made when two sequences are aligned to give maximum homology.

The term “operably linked” or “operationally linked” refers to functional linkage between a regulatory sequence (e.g., promoter, enhancer, silencer, polyadenylation signal, 5′ or 3′ untranslated region (UTR), splice acceptor, IRES, triple helix, 2A self-cleaving peptides such as F2A, E2A, P2A and T2A) and a heterologous nucleic acid sequence permitting them to function in their intended manner (e.g., resulting in expression of the latter). The term encompasses positioning of a regulatory region (sequence), a sequence to be transcribed, and/or a sequence to be translated in a nucleic acid so as to influence transcription or translation of such a sequence. The regulatory sequence can be positioned at any suitable distance from the sequence being regulated (e.g., 1 nucleotide-10,000 nucleotides). For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically includes at least a core (basal) promoter.

The term “complementary” refers to the degree of anti-parallel alignment between two nucleic acid strands. Complete complementarity requires that each nucleotide be across from its opposite. No complementarity requires that each nucleotide is not across from its opposite. The degree of complementarity determines the stability of the sequences to be together or anneal/hybridize. Furthermore various DNA repair functions as well as regulatory functions are based on base pair complementarity.

As used herein, a DNA or RNA nucleotide sequence as recited refers to a polynucleotide molecule including the indicated bases in a 5′ to 3′ direction, from left to right.

The term “CRISPR/Cas” or “clustered regularly interspaced short palindromic Repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR associated systems (Cas) that use short RNA to direct degradation of foreign nucleic acids. In bacteria, the CRISPR system provides acquired immunity against invading foreign DNA via RNA-guided DNA cleavage. The “CRISPR/Cas” system or “CRISPR/Cas-mediated gene editing” refers to a CRISPR/Cas system that has been modified for genome editing/engineering. For a type II CRISPR/Cas system, it is typically composed of a “guide” RNA (gRNA) and a non-specific CRISPR-associated endonuclease (Cas9). “Guide RNA (gRNA)” is used interchangeably herein with “short guide RNA (sgRNA)” or “single guide RNAi” (sgRNA). The sgRNA is a short synthetic RNA composed of a “scaffold” sequence necessary for Cas9-binding and a user-defined, ˜20 nucleotide “spacer” or “targeting” sequence which defines the genomic target to be modified. The genomic target of Cas9 can be modified by changing the targeting sequence present in the sgRNA.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides can be used for targeting cleaved double stranded DNA.

The term “knockdown” refers to a decrease in gene expression of one or more genes. The term “knockout”, or “KO” refers to the ablation of gene expression of one or more genes.

“Endogenous” refers to any material from or produced inside an organism, cell, tissue or system. “Exogenous” refers to any material introduced from or produced outside an organism, cell, tissue or system.

“Encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

“Tumor burden” or “tumor load” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms tumor burden can be assessed using guidelines provided in the Response Evaluation Criteria in Solid Tumors (RECIST).

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the terms “variant” or “active variant” refers to a polypeptide or polynucleotide that differs from a reference polypeptide or polynucleotide, but retains essential properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### II. Methods of Providing Isogenic Cells or Tissues Having an Ablated Chromosome or Chromosome Arm, and Cells or Tissues Created Thereby

Methods for manipulating copy numbers of chromosomes and fragments or arms thereof in cells and tissues have been established. The methods are effective to alter the number of copies of a chromosome, or a fragment thereof, such as a chromosome arm, in an aneuploid cell or tissue, or a diploid cell or tissue.

In some forms, the methods reduce the number of copies of a chromosome or a fragment thereof in a cell having multiple copies of a chromosome. For example, in some forms the described methods are used to change the number of copies of one or more chromosomes in a cell or tissue from 2, 3, 4, or more than 4 copies, to fewer copies, such as to 3 copies, or 2 copies, or one copy. For example, in other forms the described methods are used to change the number of copies of one or more chromosomes in a triploid, tetraploid or pentaploid cell or tissue, to two or one copy, effectively providing a diploid or monoploid cell or tissue derived from the aneuploid precursor cell or tissue. In some forms, the genotype of the resulting diploid-like cell or tissue is equivalent to the “parent” aneuploid cell or tissue from which it is derived, except for the lack of the additional copy or copies of the ablated chromosome or fragment thereof. Therefore, methods for providing diploid-like cells or tissues derived from aneuploid precursor cells or tissues are described.

Typically, the methods ablate a single copy of an aneuploid chromosome or fragment thereof in a cell or tissue. Therefore, the methods enable step-wise ablation of multiple copies of an aneuploid chromosome or fragment thereof in a tetrasomic cell or tissue by removing a single copy in each “step”, such that subsequent repeated “rounds” of the method are required to reduce multiple copies. In an exemplary form, the methods reduce the number of copies of a given chromosome, such as an aneuploid chromosome in a tetrasomic cell or tissue from four copies to three copies; in a further exemplary form, the methods reduce the number of copies of an aneuploid chromosome in (e.g., the same) trisomic cell or tissue from three copies to two copies; in a further exemplary form, the methods reduce the number of copies of a chromosome in (e.g., the same) diploid cell or tissue from two copies to one copy, thereby producing a monosomic cell or tissue. Thus, in some forms the methods effectively eliminate one or more than one copy of an aneuploid chromosome in a cell or tissue. In some forms, the methods provide aneuploid cells or tissues having more than two copies of a chromosome. In other forms, the methods provide diploid-like cells or tissues having two copies of a chromosome. In further forms, the methods provide monosomic cells or tissues having only one copy of a chromosome (i.e., by reducing the number of copies of a chromosome in a cell from 2 copies to 1 copy). All of the described methods can include one or more further steps of screening cells or tissues prepared according to the described methods for susceptibility to one or more active agents.

In exemplary forms, the methods provide isogenic diploid or diploid-like cells or tissues derived from an aneuploid trisomic cell or tissue having an aneuploid chromosome or part thereof, such as an aneuploid chromosome arm. Cells or tissue created according to the methods and compositions thereof are provided.

The methods typically include introducing into the aneuploid cell a gene editing composition configured to cleave the aneuploid chromosome arm. The gene editing composition cuts the aneuploid chromosome arm, rendering the aneuploid chromosome arm non-functional. In some forms, the methods also introduce into the aneuploid cell a nucleic acid that is configured to integrate into the aneuploid chromosome arm. Exemplary nucleic acids include genes, or fragments thereof, such as reporter genes, and telomere repeat sequences. The methods typically also include one or more steps for isolating the isogenic diploid-like cells.

If the cells or tissue began as aneuploid for only one chromosome, with the remaining chromosomes being disomic, the result of the methods can be fully diploid cells or tissue. For example, if the cells or tissue began as aneuploid for two or more chromosomes, with the remaining chromosomes being diploid, the result of the methods can be fully diploid cells or tissue, or cells in which fewer than all of the aneuploid chromosomes become diploid. In some forms, altered cells or tissue include one or more genetically-altered, aneuploid chromosome arm.

### A. Restoring Disomy in Aneuploid Cells or Tissue Using CRISPR Targeting (ReDACT) Methodologies

Methods for Restoring Disomy in Aneuploid cells or tissue using CRISPR Targeting (also referred as “ReDACT”) are provided. REDACT is a set of chromosome engineering tools that allows elimination of specific aneuploidies from cancer genomes.

As described in the Examples, a set of approaches collectively called ReDACT has been developed to conduct comparable cellular assays with aneuploidy. The ReDACT methods all include one or more steps for genetic engineering of aneuploid cells or tissue to disable an aneuploid chromosome, or a fragment thereof, such as an aneuploid chromosome arm within the cell or tissue, however, various concepts can be applied to identify and isolate the resulting isogenic, diploid cells or tissue, including Negative Selection (“ReDACT-NS”); and Telomere Replacement (“ReDACT-TR”). A third method uses CRISPR only (“ReDACT-CO”), without a step for integrating nucleic acids into the aneuploid chromosome or chromosome arm. Typically, the methods employ CRISPR/Cas—based genetic engineering, however any system for designing selective genetic changes in viable cells or tissue in vitro can be employed for genetic manipulation of aneuploid cells or tissue. In alternative forms, the methods apply a negative-selection (NS) system to select for cells or tissue lacking the entire aneuploid chromosome, for example, by targeted insertion of an integrant such as a lethal “suicide” gene into an aneuploid chromosome, then applying the negative selection methodology to screen for cells or tissue lacking the integrant (i.e., lacking the aneuploid chromosome).

i. ReDACT-Negative Selection (NS) Methodology

In an exemplary form of a REDACT Negative Selection (NS) method, a nucleic acid encoding a selection cassette is engineered into an aneuploid chromosome or chromosome arm. Gene editing is then used to cut the aneuploid arm between the selection cassette and the centromere. The cells are then treated with a compound toxic to cells expressing the selection cassette to select for cells that have lost the targeted aneuploid chromosome.

Exemplary reporter genes/systems that can be implemented into the described ReDACT-NS methods include Cytosine Deaminase, Diphtheria Toxin, negative selection via fluorophore (e.g., integrating a GFP cassette onto the chromosome of interest and then using FACS to isolate non-GFP-expressing cells), and ganciclovir/HSV-TK.

In an exemplary form, a reporter gene is or includes herpesvirus thymidine kinase (HSV-TK), whereby the selecting includes contacting the cells with ganciclovir in an amount and time effective to kill cells expressing the HSV-TK gene, whereby the aneuploid cells expressing HSV-TK are susceptible to ganciclovir and the isogenic control cells lacking HSV-TK are not susceptible to ganciclovir, and whereby selecting includes isolating viable cells.

a. ReDACT-NS to Remove a Chromosome Arm

In an exemplary form of the Negative Selection method, aneuploid cells or tissue are transfected herpesvirus thymidine kinase (HSV-TK), that is integrated onto a target chromosome arm, such as an aneuploid chromosome arm. The cells or tissue are then transfected with a gRNA that cuts between the integrant and the centromere to cleave and ablate the function of the target chromosome arm, such as an aneuploid chromosome arm. The cells or tissue are then treated with ganciclovir, which is toxic to cells or tissue that express HSV-TK. Loss of the target chromosome arm, such as an aneuploid chromosome harboring HSV-TK allows cells or tissue to survive ganciclovir selection. As demonstrated in the Examples, the ReDACT-NS method created isogenic diploid-like clones derived from human cell lines that had lost specific aneuploid chromosomes.

An exemplary method for ReDACT-NS to remove a target chromosome arm includes:
(i) introducing into a target cell or tissue, such as an aneuploid cell, a gene editing composition configured to cleave a target chromosome arm and integrate a nucleic acid encoding a reporter gene that is configured to integrate into the target chromosome arm;
(ii) introducing into the target cell or tissue a gene editing composition configured to cleave the aneuploid chromosome arm between the integrant and the centromere of the target chromosome arm; and
(iii) selecting isogenic cells or tissue having a cleaved and/or ablated and/or lost target chromosome arm.

Typically, when the methods target removal of a specific target chromosome arm for removal, the gene editing composition includes a CRISPR/Cas composition including a guide RNA (gRNA) configured to cleave between nucleotides within the target chromosome arm between the integrated reporter gene and a centromere of the aneuploid chromosome arm; and whereby selecting isogenic cells includes selecting cells lacking the reporter gene.

In some forms, the target cell or tissue is an aneuploid cell or tissue, for example, including one or more aneuploid chromosome arms.

An exemplary method for ReDACT-NS to remove an aneuploid chromosome arm includes:
(i) introducing into an aneuploid cell or tissue, a gene editing composition configured to cleave an aneuploid chromosome arm and integrate a nucleic acid encoding a reporter gene that is configured to integrate into the aneuploid chromosome arm;
(ii) introducing into the aneuploid cell or tissue a gene editing composition configured to cleave the aneuploid chromosome arm between the integrant and the centromere of the target chromosome arm; and
(iii) selecting isogenic cells or tissue having a cleaved and/or ablated and/or lost aneuploid chromosome arm.
   b. ReDACT-NS to Remove an Entire Chromosome

In some forms, the methods do not include the second step of expressing a crRNA configured to cut between the integrant and the centromere. Cells naturally mis-segregate chromosomes at a low level. Depending on the cell type, between 1 in 100 and 1 in 10,000 cells will lose a chromosome due to random errors in mitosis. Through the use of the selection marker, the methods isolate cells that have spontaneously lost a chromosome, such as an aneuploid chromosome, that was targeted with HSK-TK or another marker. Therefore, in some forms, the methods apply a negative-selection (NS) system to select cells or tissue lacking the entire aneuploid chromosome. For example, in some forms, the methods insert of an integrant gene, such as a lethal “suicide” gene, selectively into one or more aneuploid chromosomes, then apply negative selection to screen for cells or tissue lacking the integrant (i.e., lacking the aneuploid chromosome). In this manner, methods for restoring diploidy in trisomic or multi-omic aneuploid cells or tissue effectively identify cells or tissue lacking an entire aneuploid chromosome amongst the population of aneuploid cells or tissue.

Therefore, methods to target removal of an entire chromosome, such as an aneuploid chromosome are also provided. Typically, when the methods target removal of an entire chromosome, such as an aneuploid chromosome, the gene editing composition includes a CRISPR/Cas composition including a guide RNA (gRNA) configured to introduce an integrant for negative selection between nucleotides within a target chromosome.

When an intact chromosome is targeted the methods do not include a second CRISPR step of cutting between the integrant and the centromere; and whereby selecting isogenic cells includes selecting cells lacking the reporter gene.

An exemplary method for ReDACT-NS to remove an entire target chromosome includes:
(i) introducing into a target cell or tissue, such as an aneuploid cell or tissue, a gene editing composition configured to cleave an aneuploid target chromosome and integrate a nucleic acid encoding a reporter gene that is configured to integrate into the target chromosome; and
(ii) selecting isogenic cells or tissue having a cleaved and/or ablated and/or lost target chromosome.

Typically, when the methods target removal of a specific target chromosome for removal, the gene editing composition includes a CRISPR/Cas composition including a guide RNA (gRNA) configured to cleave between nucleotides within the target chromosome, whereby selecting isogenic cells or tissue includes selecting cells lacking the reporter gene. Therefore, in some forms, the methods produce isogenic cells lacking an entire chromosome as compared to the precursor cell or tissue. The precursor cell or tissue can be a diploid cell or tissue, triploid cell or tissue, or pentaploid, hexaploid cell, etc. Therefore, in some forms, the methods provide monoploid, diploid, triploid, pentapolid cells or tissue, etc.

In some forms, the target cell or tissue is an aneuploid cell or tissue, for example, including one or more aneuploid chromosome arms.

An exemplary method for ReDACT-NS to remove an entire aneuploid chromosome includes:
(i) introducing into an aneuploid cell or tissue, a gene editing composition configured to cleave an aneuploid chromosome and integrate a nucleic acid encoding a reporter gene that is configured to integrate into the aneuploid chromosome;
(iii) selecting isogenic cells or tissue having a cleaved and/or ablated and/or lost aneuploid chromosome.

The methods produce isogenic cells or tissue derived from aneuploid cells or tissue, but lacking an entire chromosome as compared to the precursor aneuploid cell or tissue. The precursor aneuploid cell or tissue can be a trisomic cell or tissue, or pentasomic, hexasomic cell or tissue, etc. Therefore, in some forms, the methods provide isogenic diploid-like cells or tissue derived from aneuploid cells or tissue, or trisomy-like cells or tissue derived from aneuploid cells or tissue, etc.

ii. ReDACT-Telomere Replacement (TR) Methodology

In an exemplary form of the Telomere Replacement (TR) method target cells or tissues are co-transfected with a gRNA that cuts near the centromere of a target chromosome along with a nucleic acid encoding a telomere sequence.

In some forms, the nucleic acid encodes ˜100 repeats of the human telomere seed sequence. CRISPR cleavage coupled with integration of the telomeric seed sequence leads to loss of a target chromosome or arm and formation of a de novo telomere.

An exemplary method for ReDACT-TR includes:
(i) introducing into a target cell or tissue a gene editing composition configured to cleave a target chromosome or chromosome arm and a nucleic acid including a sequence encoding a telomere repeat sequence that is configured to integrate into the target chromosome or chromosome arm; and
(ii) selecting isogenic cells or tissue having a cleaved and/or ablated and/or lost target chromosome or chromosome arm.

Typically, the gene editing composition includes a CRISPR/Cas composition configured to cleave between nucleotides proximal to a centromere in the target chromosome or arm and integrate the telomere repeat sequence. In some forms, the CRISPR/Cas composition is configured to cleave the target chromosome or arm and insert a reporter gene associated with the telomere repeat sequence, and the selecting in step (ii) includes selecting isogenic cells or tissue including the reporter gene. Thus, in some forms, selecting isogenic cells or tissue includes selecting cells or tissue including the de novo telomere by treating cells or tissue with a compound that is toxic to the cells or tissue in the absence of selection cassette. In the experiments below, a telomere seed sequence was linked with a puromycin resistance gene which provided a means for selection of aneuploidy loss cells using the drug puromycin.

In an exemplary form of the Telomere Replacement (TR) method, aneuploid cells are co-transfected with a gRNA that cuts near the centromere of an aneuploid chromosome along with a nucleic acid encoding a telomere sequence.

As demonstrated in the Examples, the ReDACT-TR method created isogenic diploid-like clones derived from human cell lines that had lost specific aneuploid chromosomes.

An exemplary method for ReDACT-TR for the ablation of an aneuploid chromosome arm includes:
(i) introducing into an aneuploid cell or tissue a gene editing composition configured to cleave an aneuploid chromosome arm and a nucleic acid including a sequence encoding a telomere repeat sequence that is configured to integrate into the aneuploid chromosome arm; and
(ii) selecting isogenic cells or tissue having a cleaved and/or ablated and/or lost aneuploid chromosome arm.

Typically, the gene editing composition includes a CRISPR/Cas composition configured to cleave between nucleotides proximal to a centromere in the aneuploid chromosome arm and integrate the telomere repeat sequence. In some forms, the CRISPR/Cas composition is configured to cleave the aneuploid chromosome arm and insert a reporter gene associated with the telomere repeat sequence, and the selecting in step (ii) includes selecting isogenic cells or tissue including the reporter gene. Thus, in some forms, selecting isogenic cells or tissue includes selecting cells or tissue including the de novo telomere by treating the cells or tissue with a compound that is toxic to the cells or tissue in the absence of selection cassette.

iii. ReDACT-CRISPR Only (CO) Methodology

In an exemplary form of the CRISPR Only (CO) method, target cells, such as aneuploid cells or tissue, such as a tissue sample from an aneuploid tumor are co-transfected with a gRNA that cuts near the centromere of a target chromosome, such as an aneuploid chromosome. In some forms, CRISPR cleavage by itself is sufficient to trigger chromosome loss, or loss of a chromosome arm. Therefore, in some forms, target cells, such as aneuploid cells or tissue, such as a tissue sample from an aneuploid tumor are transfected with a gRNA targeting a specific chromosome or chromosome arm without any other selection markers.

An exemplary method for ReDACT-CO includes:
(i) introducing into the target cell or tissue:
a gene editing composition configured to cleave a target chromosome or a target chromosome arm;
and a nucleic acid configured to integrate into the target chromosome or chromosome arm;
and
(ii) selecting isogenic cells or tissues having a cleaved and/or ablated and/or lost target chromosome or chromosome arm.

As demonstrated in the Examples, the ReDACT-CO method created isogenic diploid-like clones derived from human cell lines that had lost specific aneuploid chromosomes.

An exemplary method for ReDACT-CO for targeted removal of an aneuploid chromosome arm in an aneuploid cell or tissue includes:
(i) introducing into the aneuploid cell or tissue:
a gene editing composition configured to cleave an aneuploid chromosome arm;
and a nucleic acid configured to integrate into the aneuploid chromosome arm;
and
(ii) selecting isogenic cells or tissues having a cleaved and/or ablated and/or lost aneuploid chromosome arm.

In some forms, the step of selecting includes one or more methods of cell sequencing, cell viability, cell adhesion, and cell proliferation/reproduction assays, for example, wherein isogenic cells or tissues having a cleaved aneuploid chromosome arm include a cleaved target chromosome or arm, such as an aneuploid chromosome arm sequence, and/or reduced cell viability, and/or reduced cell adhesion, and/or reduced cell proliferation as compared to the target cells or tissues having an intact target chromosome, or arm, such as an aneuploid chromosome arm.

a. Methods for Genetic Manipulation of Target Cells or Tissue

The described methods introduce into the target cell or tissue, such as aneuploid cells or tissue) a gene editing composition configured to cleave the target chromosome arm. Preferred gene manipulation systems include protein-guided systems, such as CRISPR-Cas. Therefore, in some forms, the gene editing composition is a CRISPR/Cas-mediated genetic manipulation composition, configured to cleave the aneuploid chromosome arm. However, alternative forms of genetic manipulation are also contemplated. Exemplary alternative gene editing systems include, but are not limited, to triplex-forming oligonucleotides, pseudo-complementary oligonucleotides, zinc finger nucleases, and TALENs.

In some forms, the gene editing composition is configured to specifically and selectively cleave an aneuploid chromosome arm. In some forms, cleavage of the aneuploid chromosome arm is directed to a region proximal to the centromere of the aneuploid chromosome arm.

iv. CRISPR/Cas

In some forms, the gene editing system that induces a single or a double strand break in the target cell's genome is CRISPR/Cas, or a nucleic acid construct encoding the Cas nuclease.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing, or changing specific genes) for use in eukaryotes (see, for example, Cong, Science, 15:339(6121):819-823 (2013) and Jinek, et al., Science, 337(6096):816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some forms, a tracrRNA and crRNA are linked and form a chimeric crRNA-tracrRNA hybrid where a mature crRNA is fused to a partial tracrRNA via a synthetic stem loop to mimic the natural crRNA: tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the “target sequence” and the tracrRNA is often referred to as the “scaffold.”

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences.

In some forms, the methods employ one or more Cas nucleases that is complexed with one or more single guide RNA (sgRNA) to form CRISPR/Cas ribonucleoproteins (RNPs). In other embodiments, the methods employ one or more Cas nucleases, and/or one or more single guide RNA (sgRNA) that are administered to the same cell as separate components. The Cas can be contacted with cell as a protein, or a nucleic acid (e.g., a vector) encoding the protein. In some forms, the Cas nuclease is modified with one or more nuclear localization signals (NLS), preferably two NLS. In some forms, the Cas nuclease is a Cas9 nuclease such as Streptococcus pyogenes Cas9 nuclease, or variants thereof. The sgRNA can be in the form of an RNA, or nucleic acid encoding the sgRNA. When nucleic acids (e.g., vectors) are used to deliver the sgRNA(s) and/or Cas protein, one or more vectors can be used. Thus, sgRNA(s) and Cas protein can be encoded by the same or different vectors.

a. gRNAs

The methods employ one or more gRNAs designed to selectively cut nucleic acid sequences within an aneuploid chromosome.

Typically, the methods employ centromere-targeting CRISPR gRNAs. Typically, gRNAs are provided in CRISPR plasmids, for example, transfected into cells using any technique known in the art for the transformation of viable cells. Exemplary transfection reagents include Lipofectamine 3000 or Fugene HD.

Examples of gRNA sequences that can be used in the disclosed methods and compositions are provided in the experiments below, see e.g., Table 4A and 4B.

gRNAs can be designed according to systems known in the art for the design of sgRNAs. In an exemplary form, sgRNAs for use in the described ReDACT methods are designed using Benchling cloud-based platform software for biotechnology research and development. Design criteria include targeting a unique, non-repetitive region between the integrant and the centromere. In some forms, gRNAs are configured to perfectly match the targeted chromosome. In other forms, gRNAs are configured to target a genomic region including one or more Single Nucleotide Polymorphism (SNP), e.g., so that they specifically recognize one copy of a target chromosome, but not its homolog(s).

v. Alternative Genetic Manipulation Systems

In some forms, the methods employ one or more alternative gene editing systems. The alternative gene editing systems can be used independently or in combination with CRISPR/Cas for all or part of the described methods. Exemplary alternative gene editing systems include, but are not limited, to triplex-forming, pseudo-complementary oligonucleotides, zinc finger nucleases, and TALENs.

a. Zinc Finger Nucleases

In some forms, the gene editing system that induces a single or a double strand break in the target cell's genome is zinc finger nuclease (ZFN), or a nucleic acid construct encoding ZFN. ZFNs are typically fusion proteins that include a DNA-binding domain derived from a zinc-finger protein linked to a cleavage domain.

The most common cleavage domain is the Type IIS enzyme Fokl. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li, et al., Proc., Natl. Acad. Sci. USA 89 (1992):4275-4279; Li, et al., Proc. Natl. Acad. Sci. USA, 90:2764-2768 (1993); Kim, et al., Proc. Natl. Acad. Sci. USA. 91:883-887 (1994a); Kim, et al., J. Biol. Chem. 269:31,978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys₂His₂ zinc fingers, each of which generally recognizes three to four nucleotides in the target DNA sequence. The Cys₂His₂ domain has a general structure: Phe (sometimes Tyr)-Cys-(2 to 4 amino acids)-Cys-(3 amino acids)-Phe (sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

b. Transcription Activator-Like Effector Nucleases

In some forms, the gene editing system that induces a single or a double strand break in the target cell's genome is a transcription activator-like effector nuclease (TALEN), or a nucleic acid construct or constructs encoding TALEN. TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically, they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). U.S. Published Application No. 2011/0145940, which discloses TAL effectors and methods of using them to modify DNA. Miller et al. Nature Biotechnol 29:143 (2011) reported making TALENs for site-specific nuclease architecture by linking TAL truncation variants to the catalytic domain of Fokl nuclease. The resulting TALENs were shown to induce gene modification in immortalized human cells. General design principles for TALE binding domains can be found in, for example, WO 2011/072246.

vi. Integration of Nucleic Acids into Aneuploid cells or Tissue

In some forms, the methods include one or more steps for introducing one or more nucleic acids encoding a gene, telomere repeat, or other nucleic acid sequence into an aneuploid cell or tissue. In some forms, the step of genetic manipulation simultaneously cleaves an aneuploid chromosome and inserts one or more nucleic acids encoding a gene or fragment thereof into the genome of cells. In other forms, genetic manipulation is not simultaneous, but is step-wise. For example, in certain forms, the step of genetic manipulation includes a first or further step of introducing a nucleic acid (e.g., a gene) into the trisomic cell, and a second or further step of cleaving the aneuploid chromosome.

Typically, the one or more gene editing compositions are introduced into cells according to transfection, electroporation, or any other technique known for introducing genetic material into viable cells with the purified artificial telomere construct and centromere-targeting gRNA CRISPR plasmids using Lipofectamine 3000 for AGS cells, or Fugene HD for MCF10A cells. Telomere replacement was selected for with puromycin, and puromycin resistant cells were single cell sorted onto 96-well plates. Clonal cell lines were established and screened for targeted chromosome arm loss with TaqMan copy number assays. Chromosome elimination was confirmed through SMASH karyotyping. The aneuploidy-loss cell lines generated using this method are listed in Table 2.

a. Reporter Genes

In some forms, the methods include introducing one or more nucleic acids encoding one or more reporter genes into an aneuploid cell, for example a population of cells in culture or within a section of tissue. Exemplary reporter genes include antimicrobial resistance markers (i.e., according to the ReDACT-TR methodology), or toxin sensitivity markers (i.e., according to the ReDACT-NS methodology). An exemplary antimicrobial resistance marker is a puromycin resistance gene. An exemplary toxin sensitivity marker is the herpesvirus thymidine kinase (HSV-TK) gene. The placement and insertion sequence of nucleic acids encoding one or more reporter genes can be varied, according to the methodology that is applied. In some forms, when the methods are carried out according to the ReDACT-TR methodology, the nucleic acids encoding one or more reporter genes are incorporated into an aneuploid chromosome arm having a de novo telomere, e.g., at a position upstream of the telomere, such that the gene is expressed only in cells that are successfully modified to ablate the aneuploid chromosome arm. In some forms, when the methods are carried out according to the ReDACT-NS methodology, the nucleic acids encoding one or more reporter genes are incorporated into an aneuploid chromosome arm at a position down-stream of the intended cleavage site, such that the gene is expressed only in cells that are not successfully modified to ablate the aneuploid chromosome arm, and which still express genes present on the aneuploid chromosome arm.

Exemplary reporter genes/systems that can be implemented into the described ReDACT methods include toxic and/or sensitizing genes. Exemplary lethal or “suicide” genes are known in the art. In some forms the methods, further including exposing transformed cells of a sensitizing or activating compound, such as the use of Cytosine Deaminase, Diphtheria Toxin, and ganciclovir/HSV-TK.

In other forms, the methods for negative selection do not include introducing a toxic or lethal gene. For example, in some forms, the methods rely upon secondary detection of one or more characteristics, such as expression of a detectable marker, to implement negative selection.

In some forms, the detectable marker is a fluorophore such as GFP, Venus, mIFP, LssmOrange, TagRFP657, mOrange2, mApple, Sapphire, mTagBFP2 and mCerulean3, and the methods integrate a fluorescent protein (e.g., GFP) cassette onto the chromosome of interest and then using cell sorting and/or screening techniques, such as FACS to isolate non-fluor-expressing cells. In other forms, the methods label an expressed marker with a fluorophore and using FACS to remove the labelled cells, etc. Exemplary fluorophores that can be used include hydroxycoumarin, methoxycoumarin, aminocoumarin, Cy2, FAM, Fluorescein, FITC, HEX, Cy3, TRITC, R-phycoerythrin (PE), Rhodamine Red, Tamara, Cy3, Rox, and Allophycocyan

### i) Herpes Simplex Virus Thymidine Kinase (HSVTK)

In some forms, the reporter gene system implemented within the described methods is or includes selective expression of a lethal gene, such as a Herpes Simplex Virus Thymidine Kinase (HSVTK), followed by exposure to Ganciclovir (GCV).

HSVTK phosphorylates GCV forming GCV mono-phosphate, while the subsequent formation of GCV di- and triphosphates are catalyzed by cellular kinases. GCV triphosphate incorporates into DNA causing replication to be blocked.

Targeted suicide gene therapy based on insertion and expression of the HSVTK gene followed by exposure to Ganciclovir (GCV) is described in Poulsen, et al., Cancer Gene Ther. 2008; 15:563-75, the contents of which are hereby incorporated by reference in their entirety.

### ii) Cytosine Deaminase

In some forms, the reporter gene system implemented within the described methods is or includes selective expression of a lethal gene, such as a Cytosine Deaminase, followed by exposure to the 5-fluorocytosine (5-FC) prodrug.

The suicide gene cytosine deaminase (CD) from either bacteria or fungi converts the clinically safe and non-toxic prodrug 5-fluorocytosin (5-FC)(20) to 5-fluorouracil (5-FU), a clinically approved cytostatic. Cellular enzymes further metabolize 5-FU leading to the formation of active toxic metabolites, which block DNA and RNA synthesis by simultaneous inhibition of the thymidylate synthase and incorporation in DNA and RNA during replication and transcription.

Targeted suicide genes based on the yeast cytosine deaminase (YCD) gene alone or fused with the yeast uracil phosphoribosyl transferase (YUPRT) gene followed by administration of 5-fluorocytosine (5-FC) prodrug is described in Christensen, et al., Clin Cancer Res. 2010 16(8):2308-19, the contents of which are hereby incorporated by reference in their entirety.

iii) Diphtheria Toxin

In some forms, the reporter gene system implemented within the described methods is or includes selective expression of a lethal gene, such as a Diphtheria toxin. Diphtheria toxin is a single chain, 62 kDa protein consisting of 535 amino acid residues that is produced by Corynebacterium diphtheria containing lysogenic beta phage. DT mediates its cytolethal effect through the inhibition of protein synthesis in susceptible cells; in the cytoplasm of susceptible cells, the catalytic domain of DT first binds to nicotinamide dinucleotide (NAD) and then transfers an adenosine diphosphate ribosyl (ADPR) moiety to elongation factor 2, which subsequently inhibits protein synthesis. Exemplary Diphtheria toxins and expression systems are described in Shafiee, et al., Front Microbiol. 2019; 10:2340, the contents of which are hereby incorporated by reference in their entirety. In some forms, the genetic material that codes for the catalytic domain of DT (DTA) is delivered instead of and/or in addition to the intact DT gene.

b. Telomere Repeats

In some forms, the methods include one or more steps for introducing one or more nucleic acids encoding one or more telomere repeat sequences into an aneuploid cell (i.e., according to the ReDACT-TR methodology). In some forms, the nucleic acid is or includes a purified artificial telomere construct. In some forms, the ReDACT-TR methods include one or more steps to integrate a nucleic acid encoding a telomere seed sequence into trisomic cells to induce loss of trisomy. In some forms, the methods introduce the telomere repeat sequence into trisomic cells together with one or more selection markers. An exemplary selection marker is an antibiotic resistance gene. Therefore, in exemplary forms, the methods transfect a plasmid linking a telomere seed sequence with an antibiotic resistance gene selection marker into trisomic cells. An exemplary antibiotic resistance gene is a puromycin resistance gene. Therefore, in some forms, the methods transfect a plasmid linking a telomere seed sequence with a selection marker such as a puromycin resistance gene into the trisomic cells.

vii. Selection of Genetically Modified Cells

In some forms, the methods for Restoring Disomy in Aneuploid cells using CRISPR Targeting (termed “ReDACT”) include one or more steps for selecting and/or isolating the genetically modified cells that result from the described methods of genetic manipulation to selectively cleave an aneuploid chromosome or fragment thereof, such as a chromosome arm. In an exemplary form, the genetically-modified cells produced according to the described ReDACT methods are diploid-like cells having two copies of a chromosome.

In some forms, the methods for genetic engineering to disable an aneuploid chromosome arm in a population of aneuploid (e.g., trisomic) cells are 100% efficient. However, in other forms, methods for genetic engineering to disable an aneuploid chromosome arm in a population of aneuploid (e.g., trisomic) cells are less than 100% efficient, for example, in some forms, only a proportion of the total number of aneuploid (e.g., trisomic) cells is effectively manipulated for cleavage of the aneuploid chromosome arm, such as 90%, 80%, 70%, 60%, 50%, 40%, 30%, 20%, 10%, or less than 10% of the total number of aneuploid (e.g., trisomic) cells is effectively manipulated for cleavage of the aneuploid chromosome arm. Therefore, in some forms, the methods create a mixture of aneuploid (e.g., trisomic) and disomic cells. Methods for selecting and isolating disomic cells from a mixture of aneuploid (e.g., trisomic) and disomic cells are described.

Typically, methods for selecting and/or isolating distinguish the genetically modified diploid-like cells that result from successful genetic manipulation to disable the aneuploid chromosome arm from the “parent” aneuploid cells. Selection systems based on the absence or presence of reporter genes and the expressed reporter proteins are well known in the art.

a. Sequence Analysis

In some forms, the methods for selecting and/or isolating the genetically modified diploid-like cells that result from the described methods of genetic manipulation to selectively cleave an aneuploid chromosome arm include sequence analysis to determine DNA copy number. As demonstrated in the Examples, chromosome loss causes a substantial downregulation of genes encoded on an aneuploid chromosome, though these effects can be buffered to some extent by cellular dosage compensation. Therefore, in some forms, the methods include one or more steps to assess DNA copy number in cells to select disomic cells from a mixture of aneuploid (e.g., trisomic) and disomic cells. An exemplary system for assessing DNA copy number includes the SMASH-Seq program.

In some forms, the methods for sequence analysis include heterozygosity analysis. In an exemplary form, the methods identify one or more single nucleotide polymorphisms (SNPs) on the aneuploid chromosome that are heterozygous in the parental aneuploid (e.g., trisomic) cell line and that exhibit loss-of-heterozygosity (LOH) following ablation of the aneuploid chromosome. Therefore, in some forms, the methods assess the heterozygosity of one or more SNPs to identify disomic and aneuploid (e.g., trisomic) cells.

b. Proliferation Assays

In some forms, the methods for selecting and/or isolating the genetically modified diploid-like cells include a cell proliferation assay(s), for example, to measure the doubling time of aneuploid (e.g., trisomic) and the disomic cells in culture. As demonstrated in the Examples, it has been established that aneuploidy-loss clones divide more slowly in vitro compared to the 1q-trisomic cells. In an exemplary form, trisomic cells have a doubling time that is at least ˜35% greater than that of isogenic disomic cells. Therefore, in some forms, the methods include a cell proliferation assay to select disomic cells from a mixture of aneuploid (e.g., trisomic) and disomic cells.

c. Negative Selection of Disomic Cells

In some forms, the methods for selecting and/or isolating the genetically modified diploid-like cells include one or more steps to negatively-select cells that lack a reporter gene. In preferred forms, the negative selection includes a toxin-sensitivity gene that is, for example, engineered into the aneuploid chromosome arm. When the methods include insertion of a toxin-sensitivity gene into the aneuploid chromosome arm, one or more steps for selection of disomic cells from a mixture of aneuploid (e.g., trisomic) and disomic cells includes culturing the cells in the presence of the toxin to which only the aneuploid (e.g., trisomic) cells are sensitive. An exemplary toxin-sensitivity gene is the herpesvirus thymidine kinase (HSV-TK) gene. Ganciclovir is toxic to cells that express HSV-TK. Therefore, in some forms, when the methods engineer the HSV-TK gene selectively into the aneuploid chromosome arm, the methods include one or more steps to culture cells in the presence of a suitable amount of Ganciclovir to kill cells expressing HSV-TK. In some forms, the methods select and/or isolate disomic cells from a mixture of aneuploid (e.g., trisomic) and disomic cells in the presence of Ganciclovir for a time sufficient to kill cells that express the HSV-TK, while permitting modified disomic cells lacking this gene to survive.

d. Positive Selection of Aneuploid (e.g., Trisomic) Cells

In some forms, the methods for selecting and/or isolating the disomic cells include one or more steps to positively select the aneuploid (e.g., trisomic) cells, for example, cells including one or more reporter genes that are engineered selectively into the aneuploid chromosome arm. These cells can be isolated from the pool of disomic and aneuploid (e.g., trisomic) cells, for example, based on cell-sorting techniques or other cell selection methods, and the remaining cells are the disomic cells, lacking the reporter gene on the aneuploid chromosome arm/expressed gene product.

e. De Novo Telomere-Based Selection of Disomic Cells

In some forms, when the methods include insertion of a telomere repeat sequence in place of the aneuploid chromosome arm, one or more steps for selection of disomic cells from a mixture of aneuploid (e.g., trisomic) and disomic cells includes positive selection of disomic cells, for example, including and/or expressing a reporter gene expressed only by the disomic cells. In some forms, as described in the Examples, the reporter gene is linked to the telomere repeat sequence. An exemplary reporter gene is an antimicrobial resistance gene. Therefore, in some forms, the methods select disomic-like cells having a de novo telomere by positive selection for cells expressing a selection cassette (e.g., antimicrobial resistance gene) that is associated with the de novo telomere. For example, in some forms selection includes culturing the cells in the presence of the antimicrobial for a time sufficient to kill aneuploid (e.g., trisomic) cells that do not express the antimicrobial resistance gene, whilst permitting modified disomic cells having a de novo telomere and the resistance gene to survive. An exemplary antimicrobial resistance gene is a puromycin resistance gene. In an exemplary form, selection includes culturing the cells in the presence of puromycin for a time sufficient to kill aneuploid (e.g., trisomic) cells that do not express the puromycin resistance gene, whilst permitting modified disomic cells having a de novo telomere and the puromycin resistance gene to survive.

### III. Compositions of Cells

Compositions of cells including a modified number of chromosomes derived according to the described methods are provided. The modified cells can include 1, 2, 3, 4, or more copies of a chromosome or fragment thereof, such as an aneuploid chromosome arm. Disomic cells derived from aneuploid (e.g., trisomic) cells are provided. Generally, disomic cells derived from aneuploid (e.g., trisomic) cells are isogenic with the aneuploid “parent” cells from which they are derived, but have at least one ablated aneuploid chromosome arm. In some embodiments, the aneuploid cells are aneuploid for one chromosome, and the disclosed methods induce formation fully diploid cells (i.e., fully cured of aneuploidy). In other embodiments, the parent cells are aneuploid for two or more chromosomes, and the disclosed method induce reduction of aneuploidy relative to the parent cells. Such cells can be fully diploid cells (i.e., fully cured of aneuploidy) or partially diploid cells provided they are less aneuploidy than the parent (i.e., partially cured of aneuploidy). Cells modified to reduce aneuploidy including both fully and partially diploid cells are referred to herein as diploid-like cells, even where they are still aneuploidy.

In some forms, aneuploid (e.g., trisomic) cells are obtained from a human subject. Therefore, human aneuploid cells expressing and/or including an aneuploid chromosome are described. In some forms, cells are obtained from a subject having little or no symptoms of a disease, and/or who has not been diagnosed with a disease. In other forms, cells are obtained from a subject identified as having or at increased risk of having a disease or disorder, such as cancer. Therefore, in some forms, the human aneuploid cells are tumorigenic or cancer cells. In some forms the cells are immortalized before genetic manipulation. Thus, the parent cells can be a cell line, e.g., a commercially available cell line.

In some forms, the cells are frozen prior to or after genetic manipulation according to the described methods. Methods and compositions for freezing and thawing viable eukaryotic cells are known in the art. In some forms, the cells are viable, thawed cells.

In preferred forms, the introduction of gene editing compositions, such as CRISPR/Cas to the cells occurs through genetic modification of the cells. In some forms, genetic modification of the cell includes introduction of nucleic acids, such as genes or vectors encoding genes to the cell for expression of the genes within the cell. Therefore, genetically modified (transgenic) cells according to the described methods are provided.

Compositions including disomic cells derived from aneuploid (e.g., trisomic) cells together with one or more reagents, excipients, culture media, or other components are also provided. Typically, the disomic cells derived from aneuploid (e.g., trisomic) cells are produced according to the described methods for ablation of an aneuploid chromosome in “parent” aneuploid/trisomic cells.

### A. Precursor Cells and Tissues

Precursor cells and tissues are provided. Typically, the described methods ablate a chromosome or fragment thereof, such as a chromosome arm, from a precursor cell or tissue to provide an isogenic cell or tissue derived from the precursor cell or tissue. Precursor cells can be in the form of a population of cells, such as a homogeneous population of cells or a heterogeneous population of cells, or cells within a tissue or organoid. The cells or tissue can be diseased cells or tissue, such as cancerous cells or tissue. Exemplary tissues include a section or sample from one or more organs of the body. Methods of obtaining, maintaining and storing tissue and biological samples are known in the art. In some forms, the tissue includes mixtures of cell types, having the same or different disease state. For example, in some forms, the tissue includes a mixture of cancerous and non-cancerous cells. In some forms, the tissue includes a mixture of aneuploid and diploid cells. In some forms, the precursor tissue and/or cells are present in the form of a tissue graph, prosthesis or organoid.

Exemplary cells and tissue for use as precursor cells or tissue in the described methods include aneuploid cells and tissue. Therefore, aneuploid cells and tissues are provided. Typically, aneuploid “parent” cells that are useful in of the described methods for aneuploidy reversion include one or more aneuploid chromosomes.

By definition, aneuploid cells have an abnormal number of chromosomes. Each chromosome contains hundreds of genes and the addition or loss of a single chromosome arm disrupts the existing equilibrium in cells. Aneuploidy typically occurs during meiosis when pairs of chromosomes do not complete the correct process of cell division and fail to separate. Each species of plants and animals has a set number of chromosomes. Humans have 23 pairs of chromosomes, for a total of 46 chromosomes. Typically, the aneuploid cell is a human aneuploid cell, e.g., including a doubling or additional component of any one of the 23 chromosomes.

In other forms, the methods provide modified organoids and patient-derived xenografts, in addition to cell lines.

### 1. Trisomic Arm Aneuploidy

Each chromosome is divided into two sections (arms) based on the location of a narrowing (constriction) called the centromere. By convention, the shorter arm is called p, and the longer arm is called q. The chromosome arm is the second part of a gene's address. For example, 5q is the long arm of chromosome 5, and Xp is the short arm of the X chromosome. This nomenclature is also used herein to identify the location(s) of aneuploidy.

For example, in some forms, the aneuploid cells are trisomic cells, including an additional copy of a q arm of one or more chromosomes as compared with wild-type, disomic cells. Additionally or alternatively, the aneuploid cells are trisomic cells, including an additional copy of a p arm of one or more chromosomes as compared with wild-type, disomic cells.

Chromosome arm aneuploidy (CAA) is characterized by copy number gain or loss of chromosome arms. Aneuploid cells can include an additional aneuploid chromosome p and/or q arm associated with any one or more of chromosomes 1-22, X, and/or Y. Some cells have combinations of two, three, four, or more different aneuploid arms.

In some forms, the aneuploid cell includes gain of one or more of chromosomes 1, 2, 3, 4, 5, 6, 7, 8, 9, 10, 11, 12, 13, 14, 15, 16, 17, 18, 19, 20, 21, 22, X, or Y. In some embodiments the aneuploid cell includes gain of one or more chromosome arms, including one or more of 1q, 1p, 2q, 2p, 3q, 3p, 4q, 4p, 5q, 5p, 6q, 6p, 7p, 7q, 8p, 8q, 9p, 9q, 10p, 10q, 11p, 11q, 12p, 12q, 13p, 13q, 14q, 14p, 15q, 15p, 16p, 16q, 17p, 17q, 18p, 18q, 19q, 19q, 20q, 20p, 21q, 21p, 22q, 22p, X, or Y.

In particular forms, the aneuploid cell includes gain of one or more chromosome arms selected from 1q, 1p, 2q, 4q, 5q, 6p, 7p, 7q, 8q, 9q, 10q, 11q, 12p, 16p, 16q, 17p, 17q, 19q, 19q, or 21q.

In some forms, one or more genes are expressed or aberrantly expressed by a cell having an additional copy of a chromosome or part thereof. In an exemplary form, an aneuploid cell over-expresses one or more genes on an additional copy of a chromosome as compared to a corresponding disomic cell that does not include an additional copy of the chromosome. Exemplary genes that are over-expressed in trisomic cells as compared with disomic cells include, but are not limited to CYP2J2, MFSD2A, SLC16A1, UGT1A10, DCK, SLC26A2, SLC29A1, ACTB, ABCB1, SLC39A4, UCK1, UCK2, ADK, FADS2, SLC35F2, PDE3A, ABCC1, SULT1A1, NQO1, SLC47A1, SLFN11, SLFN12, DCAF15, SLC6A16 and SLC19AL. In some forms, the one or more genes that is over-expressed in trisomic cells as compared with disomic cells include genes that encode or express importer pumps and/or enzymes.

In some forms, the trisomic cell has a 1q aneuploidy, or 7p aneuploidy, or an 8q aneuploidy.

In some forms, the cell having a q arm and/or p arm aneuploidy is a cancer cell.

### 2. Trisomic Aneuploid Cancer Cells

In some forms, the aneuploid “parent” cells for use in the described methods are trisomic cancer cells, for example, aneuploid cancer cells including an additional chromosome arm on one or more chromosomes.

Around 90% of tumors have cancer cells with extra or missing chromosomes (aneuploidy), and CAA is the most prevalent type of genomic alteration in solid tumors, occurring in 90% of cases. Both hematological and solid cancers initially gain chromosome arms, while only solid cancers subsequently preferentially lose multiple arms.

Both aneuploidy burden as well as specific CAAs are associated with clinical outcome in a number of cancers. Chromosomes 1q, 6p, 7, 8q, 17, and 20q are frequently gained in CMM, encompassing oncogenes such as MYC, CDK4, MITF, MDM2, and CCND1, while chromosomes 6q, 8p, 9p, and 10q are usually lost, including the tumor-suppressor genes P14, P15, P16, and PTEN.

Trisomic aneuploid cancer cells for use in the described methods for reversion of trisomic cells to diploid-like cells can be derived from any tumor type, and/or can be derived from a biological sample obtained from a subject having cancer. The cancer can be of any type, including but not limited to leukemias including, but not limited to acute lymphoblastic leukemia, acute myeloid leukemia, myelodysplastic syndromes, chronic lymphocytic leukemia, chronic myelogenous leukemia, hairy cell leukemia; polycythemia vera; lymphomas including, but not limited to, Burkitt's lymphoma, follicular lymphoma, Hodgkin's lymphoma, non-Hodgkin's lymphoma; multiple myelomas including, but not limited to, monoclonal gammopathy of undetermined significance (MGUS), isolated plasmacytoma, extramedullary plasmacytoma, smoldering multiple myeloma, Waldenstrom's macroglobulinemia; bone cancers including, but not limited to, chondrosarcoma, chordoma, Ewing's sarcoma, osteosarcoma, giant cell tumor of bone, fibrosarcoma; soft-tissue sarcomas including, but not limited to, angiosarcoma (hemangiosarcoma), desmoplastic small round cell tumors, Kaposi's sarcoma, gastrointestinal stromal tumor, leiomyosarcoma, liposarcoma, lymphangiosarcoma, myxosarcoma, rhabdomyosarcoma, synovial sarcoma; brain tumors and nervous system cancers including, but not limited to, astrocytoma, chordoma, gliomas, ependymomas, glioblastoma, gliomas, medulloblastoma, malignant peripheral nerve sheath tumors, neuroblastoma, optic nerve glioma, pineoblastoma, primary brain lymphoma, primitive neuroectodermal tumor; breast cancers including, but not limited to, inflammatory breast cancer, invasive ductal carcinoma, invasive lobular carcinoma, medullary breast cancer, mucinous breast cancer, Paget's disease, Phyllode's tumor, tubular breast cancer; adrenal cancer including, but not limited to, adrenocortical carcinoma, pheochromocytoma; thyroid cancer including, but not limited to, anaplastic thyroid cancer, follicular thyroid cancer, Hurthle cell thyroid cancer, medullary thyroid cancer, papillary thyroid cancer; parathyroid cancer including, but not limited to, parathyroid carcinoma; pancreatic cancer including, but not limited to, adenocarcinoma, acinar cell carcinoma, pancreatoblastoma, pancreatic neuroendocrine neoplasms; ocular cancers including, but not limited to, ocular melanoma, retinoblastoma; vaginal cancers including but not limited to, vaginal adenocarcinoma, clear cell adenocarcinoma, squamous cell carcinoma, germ cell tumors, rhabdomyosarcoma, vaginal melanoma; vulvar cancer including but not limited to, adenocarcinoma, Bartholin gland carcinoma, basal cell carcinoma, squamous cell carcinoma; cervical cancers including, but not limited to, adenocarcinoma, squamous cell carcinoma; endometrial cancers including, but not limited to, endometrioid adenocarcinoma, serous carcinoma, clear cell carcinoma, mucinous carcinoma, mixed or undifferentiated carcinoma, endometrial stromal sarcoma; uterine cancers including, but not limited to, uterine sarcoma; ovarian cancers including, but not limited to, epithelial ovarian carcinomas, primary peritoneal carcinoma, fallopian tube cancer, germ cell tumors, stromal tumors, ovarian sarcoma, borderline tumor; esophageal cancers including, but not limited to, squamous cell carcinoma, adenocarcinoma, small cell carcinoma; gastric cancers including, but not limited to, adenocarcinoma, lymphoma, gastrointestinal stromal tumors, neuroendocrine neoplasms, hereditary diffuse gastric cancer, familial intestinal gastric cancer; small bowel cancer including, but not limited to, adenocarcinoma, neuroendocrine neoplasm, lymphoma, gastrointestinal stromal tumor; appendiceal cancers including, but not limited to, adenocarcinoma, neuroendocrine neoplasms; colon cancers including, but not limited to, adenocarcinoma, neuroendocrine neoplasm, lymphoma, squamous cell carcinoma; rectal cancers including, but not limited to, adenocarcinoma, neuroendocrine neoplasm, lymphoma; anal cancers including, but not limited to, squamous cell carcinoma, adenocarcinoma, small cell carcinoma, melanoma; liver cancers including, but not limited to, hepatocellular carcinoma, angiosarcoma, hemangiosarcoma, and hepatoblastoma; gallbladder cancers including, but not limited to, adenocarcinoma, squamous cell carcinoma; cholangiocarcinoma including, but not limited to, intrahepatic cholangiocarcinoma, perihilar cholangiocarcinoma, distal cholangiocarcinoma; lung and pleural cancers including but not limited to, non-small cell lung cancer, squamous cell carcinoma, adenocarcinoma, large-cell carcinoma, small-cell lung cancer, mesothelioma; testicular cancers including, but not limited to, germ cell tumor, sex-cord stromal tumor, lymphoma; prostate cancers including, but not limited to, adenocarcinoma, squamous cell carcinoma, transitional cell carcinoma, neuroendocrine neoplasm, sarcoma; penile cancer including, but not limited to, squamous cell carcinoma, melanoma, basal cell carcinoma, adenocarcinoma, sarcoma; kidney or ureteral cancer including, but not limited to, renal cell carcinoma, transitional cell cancer, squamous cell carcinoma, adenocarcinoma, sarcoma, lymphoma, Wilms tumor, neuroendocrine neoplasm, carcinosarcoma; bladder cancer including, but not limited to, transitional cell carcinoma, squamous cell carcinoma, adenocarcinoma, carcinosarcoma, small cell carcinoma; oropharyngeal cancers including, but not limited to, squamous cell carcinoma, verrucous carcinoma; basal cancers, melanoma; salivary gland cancers including, but not limited to, adenocarcinoma, mucoepidermoid carcinoma, and adenoidcystic carcinoma; skin cancers including, but not limited to, basal cell carcinoma, squamous cell carcinoma, Merkel cell carcinoma, and melanoma.

In certain forms, a trisomic aneuploid cancer cell is derived from a subject having a cancer selected from ovarian cancer, prostate cancer, acute myeloid leukemia, endometrial carcinoma, gastric cancer, melanoma pancreatic cancer, non-small cell lung cancer, Adrenocortical carcinoma, sarcoma, renal cancer, breast cancer and colorectal cancer.

In certain forms, a trisomic aneuploid cancer cell includes an additional 1q and/or 8q chromosome arm, and genetic modified cells are 1q-disomic and/or 8q-disomic cells.

i. Biological Samples

Biological samples including aneuploid (e.g., trisomic) cancer cells are described. In some forms, the aneuploid “parent” cells for use in the described methods are trisomic cancer cells, for example, aneuploid cancer cells including one or more additional chromosome arms, that are isolated from a biological sample. Exemplary biological samples include tumor biopsies from a subject having cancer. Methods of isolating viable tumor cells from a biological sample are known in the art.

B. Isogenic Cells and Tissues Derived from Aneuploid Cells

Disomic-like cells derived from aneuploid (e.g., trisomic) cells according to the described methods, and compositions thereof are provided. Typically, the disomic cells are genetically equivalent to the aneuploid (e.g., trisomic) cells from which they are derived, albeit lacking a functional aneuploid chromosome arm. Therefore, diploid-like cells derived from aneuploid “parent” cells according to the described “ReDACT” methods are termed “isogenic” with respect to the aneuploid (e.g., trisomic) cells from which they are derived. These isogenic cells have a “diploid-like” genotype, since they typically possess a genetically-engineered genome, for example, including a cleaved aneuploid chromosome arm. In some forms, the isogenic diploid-like cells include one or more recombinant nucleic acids, such as one or more engineered reporter genes, and/or one or more engineered de novo telomeres.

The isogenic diploid-like cells are particularly useful in methods for detecting aneuploid-specific genotypic and/or phenotypic features of aneuploid cells. For example, in some forms, the isogenic diploid-like cells are less sensitive to one or more chemotherapeutic agents than the corresponding “parent” cells. Therefore, in some forms, the isogenic diploid-like cells are useful in methods for screening for effective aneuploid-specific chemotherapy agents. In some forms, the isogenic diploid-like cells are derived from aneuploid cancer cells. In some forms, the isogenic diploid-like cells share some phenotypic features with the parent aneuploid cells from which they are derived but differ from the parent cells in one or more measurable ways. For example, in some forms, the isogenic diploid-like cells have a reduced doubling rate as compared with the parent cells. Typically, the isogenic diploid-like cells exhibit reduced expression of one or more genes relative to the parent cells.

Exemplary, non-limiting diploid-like cells are those formed from a trismic parental cell line, such as A2058, A2780, AGS, MCF10A, or HCT116.

In more particular embodiments, diploid-like cells are:
mutant A2058 cells having loss of a trisomic chromosome relative to A2058 parental cells, optionally loss of 1q trisomy, 7p trisomy, 8q trisomy, or a combination thereof;
mutant A2780 cells having loss of a trisomic chromosome relative to A2780 parental cells, optionally loss of 1q trisomy;
mutant AGS cells having loss of a trisomic chromosome relative to AGS parental cells, optionally loss of 1q trisomy;
mutant MCF10A cells having loss of a trisomic chromosome relative to MCF10A parental cells, optionally loss of 1q trisomy; or
mutant HCT116 cells having loss of a trisomic chromosome relative to an HCT116 parental cells, optionally loss of 8q trisomy.

Exemplary diploid-like cells are provided in the experiments below. Thus, in specific embodiments, the diploid-like cells are a clone selected from the clones of Table 2 below.

### 1. Cells and Cell Lines

Compositions of isogenic cells and cell-lines including the described chromosome-altered cells are provided. In some forms, the cells produced according to the described methods are diploid-like cells. Therefore, diploid-like isogenic cells derived from aneuploid cells are provided. In some forms, the cells produced according to the described methods are trisomic-like cells (i.e., having three copies of a chromosome or a fragment thereof, such as a chromosome arm). Therefore, trisomic isogenic cells derived from aneuploid cells, such as tetrasomic cells, are provided. In some forms, the cells produced according to the described methods are tetrasomic-like cells (i.e., having four copies of a chromosome or a fragment thereof, such as a chromosome arm). Therefore, tetrasomic isogenic cells derived from aneuploid cells, such as pentasomic cells, are provided. In some forms, the cells produced according to the described methods are pentasomic-like cells (i.e., having five copies of a chromosome or a fragment thereof, such as a chromosome arm). Therefore, pentasomic isogenic cells derived from aneuploid cells, such as hexasomic cells, are provided. In other forms, the cells produced according to the described methods are monosomic-like cells (i.e., having one copy of a chromosome or a fragment thereof, such as a chromosome arm). Therefore, monosomic isogenic cells derived from diploid cells, are provided. In some forms, the isogenic cells are expanded to provide populations of isogenic cells, and/or cultures of isogenic cells. In some forms, the viable isogenic cells are frozen, for example, in a suitable freezing medium for maintaining viable, frozen cells. In other forms, the described isogenic cells and cell lines derived from aneuploid cells can be formulated into compositions, such as pharmaceutical compositions, implants, grafts and organoids.

2. Biological implants

Biological implants, such as tissue grafts, prostheses and body fluids including the described isogenic cells are described.

Grafting refers to a surgical procedure to move tissue from one site to another on the body, or from another creature, without bringing its own blood supply with it. Instead, a new blood supply grows in after it is placed. Tissue grafts can be categorized as autograft, allograft, or xenograft. Autograft is tissue moved from one location within the body to another. An example is the use of the semitendinosus tendon from a patient to repair an anterior cruciate tendon in the same patient. Allograft is when tissue is donated from one person to use for tissue reconstruction and repair in another person. This tissue may be fresh (e.g., fresh osteochondral grafts for the osteochondral autograft transfer system [OATS]procedure), frozen (e.g., frozen cortical struts to augment repair of a fracture), or processed (e.g., demineralized bone pastes or gels). Allograft tissues undergo rigorous screening, processing (washing, irradiation), and robust testing to decrease the potential for disease transmission. Allograft tissues are obtained from licensed tissue banks throughout the United States.

Many tissue grafts and prostheses are known in the art, including connective tissue grafts, free soft tissue autogenous grafts, double papillae subepithelial connective tissue grafts, and pedicle grafts. The described isogenic cells derived from aneuploid cells can be incorporated into any tissue grafts or prostheses. Therefore, autograft tissue grafts including isogenic cells derived from aneuploid cells are described. Allograft tissue grafts including isogenic cells derived from aneuploid cells are also provided. Xenograft tissue grafts including isogenic cells derived from aneuploid cells are also provided. In an exemplary form, a patient-derived xenograft includes the described isogenic cells derived from aneuploid cells.

### 3. Organoids/Organ-On-a-Chip Models

Organoids including the described isogenic cells derived from aneuploid cells are also described.

Organoids are simple tissue-engineered cell-based in vitro models that recapitulate many aspects of the complex structure and function of the corresponding in vivo tissue. Data that cannot be readily obtained from published scientific articles, experiments, and/or gathered from animal model(s) can be obtained from in vitro model(s), including organoids and organ-on-a-chip models. Compositions and methods of creating, using and maintaining organoids are known in the art. An exemplary organoid is a human organoid that is constructed from, or includes a percentage of the described isogenic cells derived from aneuploid cells. In an exemplary form, a human intestinal organoid, breast tissue organoid, lung tissue organoid or prostate tissue organoid can be established using biopsied human tissues from patients who underwent surgical operations, and supplemented with or created using the described isogenic cells derived from aneuploid cells. In some forms, organoids are created to simulate a diseased tissue, such as a cancer, in a specific organ, e.g., prostate, breast, lung, etc., created in duplicate, e.g., with aneuploid cells or isogenic cells derived from aneuploid cells.

### IV. Methods of Screening for Aneuploid-specific Chemotherapeutic Agents

Methods for screening of aneuploid cancer cells for susceptibility to an active agent, such as a chemotherapy agent, are provided. The screening methods employ isogenic diploid-like cells derived from the aneuploid cells to identify agents that have aneuploid-specific cytotoxicity. Therefore, methods for detecting aneuploid-specific anti-cancer active agents are provided. An exemplary method includes one or more steps of:
(a) contacting a viable aneuploid cancer cell with one or more active agents under conditions suitable for cell culture,
whereby the aneuploid cell includes an additional copy of a chromosome relative to an isogenic control cell,
whereby the additional chromosome includes one or more expressed genes,
whereby the one or more expressed genes are expressed to greater extent in the aneuploid cancer cell relative to the control cell; and
(b) selecting the one or more active agents as selectively toxic to the aneuploid cancer cell when the agent reduces the viability of the aneuploid cancer cell,
whereby the reduction in viability of the aneuploid cancer cell is greater than the reduction in viability of the isogenic control cell contacted by the same active agent(s) under the same conditions. The screening methods typically include an isogenic control cell that lacks the additional chromosome that is derived from the aneuploid cancer cell by selective inactivation of a single aneuploid chromosomal arm, for example, according to one or more of the described ReDACT methods.

### A. Screening Active Agents

Methods of screening aneuploid and isogenic cells derived therefore for susceptibility and/or resilience to one or more active agents, such as one or more drugs, are described. Typically, the methods of screening include contacting a viable aneuploid cancer cell with one or more active agents under conditions suitable for cell culture, such that the effects of the active agent upon the viability, reproduction and other characteristics of the viable cell can be identified and quantified. Typically, the methods assess effects of an active agent in both aneuploid cells, such as aneuploid cancer cells, as well as isogenic (e.g., diploid-like) cells derived from the aneuploid cells according to the described ReDACT methods. For example, in some forms, the methods screen toxicity of one or more active agents in a sample culture of viable aneuploid (e.g., trisomic) cancer cells and in a sample culture of viable isogenic diploid-like cells derived from the aneuploid cells, wherein the culture conditions and/or number of cells and/or other conditions are maintained in both samples.

In some forms, the methods select an active agent as having efficacy against the trisomic cancer cells when the agent has greater toxic effect upon the trisomic cells than on the isogenic diploid-like cells derived from the trisomic cells. For example, in some forms, the methods select the one or more active agents as selectively toxic to the aneuploid cancer cell when one or more active agent(s) reduces the viability of the aneuploid cancer cell to a greater extent than the isogenic diploid-like cells under the same conditions.

### 1. Candidate Active Agents

Compositions of candidate aneuploid-specific active agents are provided for use in the described methods for drug screening. It has been established that aneuploid cancer cells exhibit aberrant expression of genes associated with an aneuploid chromosome. Therefore, in some forms, candidate active agents are active agents that are known to exert a biological effect through the one or more aberrantly-expressed genes characteristic of the phenotype of the aneuploid target cell.

In some forms, candidate active agents are active agents that are known to exert a biological effect through one or more pathways or cellular processes that are characteristic of the phenotype of the aneuploid target cell, such as an aneuploid cancer cell. For example, the experiments below exploit overexpression of genes and/or proteins in the aneuploid cells relative to isogenic, (e.g., diploid-like) cells derived from the aneuploid (e.g., trisomic) cells. Thus, in some forms, candidate active agents are active agents that exert a biological effect through the one or more under-expressed genes and/or genetic pathways in the isogenic, (e.g., diploid-like) cells derived from the aneuploid (e.g., trisomic) cells. Typically, the candidate active agents do not exert the same, or any biological effect on isogenic, (e.g., diploid-like) cells derived from the aneuploid (e.g., trisomic) cells according to the described ReDACT methods. Exemplary candidate active agents include small molecules, proteins, peptides, nucleic acids, lipids, saccharides, or polysaccharides. In some forms, the methods screen a single candidate active agent for selective cytotoxicity against an aneuploid (e.g., trisomic) cell. In other forms, the methods screen a combination of two or more candidate active agents for selective cytotoxicity against an aneuploid (e.g., trisomic) cell.

In some forms, candidate active agents include one or more known chemotherapy agents. Exemplary chemotherapy agents include, but are not limited to amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5, and combinations thereof. In other forms, the candidate agents are not known to have cytotoxicity against cancer cells, or are not known to have selective cytotoxicity when combined with one or more other active agent(s).

In some forms, the methods screen one or more candidate active agents that are known to have cytotoxicity against one or more specific cellular or molecular targets. Therefore, in some forms, the methods select a subset of active agents that target one or more cellular and/or biomolecular pathways for a first screen. In some forms, the methods perform sequential rounds of screening using one or more libraries of molecules, for example, having similar or overlapping known methods of activity and/or therapeutic activity, and/or similar or overlapping molecular structures. Classes of active agents that can be used as candidate agents to screen for efficacy in selectively reducing viability of aneuploid cells include classes of chemotherapy drugs including alkylating agents, such as chlorambucil, cyclophosphamide, cisplatin, and carboplatin; nitrosoureas, such as carmustine and lomustine, anti-metabolites, such as fluorauracil, methotrexate and fudarabine; plant alkaloids and natural products such as vincristine, paclitaxel, and topotecan; anti-tumor antibiotics such as bleomycin, doxorubicin and mitoxantrone; hormonal agents such as corticosteroids (e.g., prednisone and dexamethasone), sex hormones such as tamoxifen and leuprolide; and biological response modifiers, such as herceptin and avastin, erbitux and rituxan.

In certain forms, the candidate active agent is one or more of austocystin D, tunicamycin, 3-bromopyruvate, Curcumin, Gemcitabine, decitabine, bis(maltolato)oxovanadium(IV), Gemcitabine, atorvastatin, simvastatin, pitavastatin, lovastatin, fluvastatin, tepoxalin, AICAR, 6-azacytidine and 5-methylcytidine, Triciribine, ML239, YM155, DCEBIO, zardaverine, Dasatinib, Oncrasin-1, HSP90 inhibitors, Mitoxantrone, disulfiram, DCEBIO, zardaverine, Indisulam, Temsirolimus, RX-3117, 3-deazauridine, Methotrexate, and CGS-15943.

In some forms, the methods screen a multiplicity of candidate active agents for efficacy (e.g., selective cytotoxicity of aneuploid cells) to identify potentially effective chemotherapeutic agents and/or combinations of agents for use in aneuploid cancers. Therefore, in some forms, the methods include one or more steps for selecting one or more candidate active agent(s) as effective when the candidate active agent(s) exert an aneuploid-specific (e.g., trisomic cell-specific) effect, such as reduced viability, reduced rate of reproduction, reduced adherence to a substrate, reduced cell size and/or reduced cell number. In some forms, the methods select one or more candidate active agent(s) as effective when the candidate active agent(s) exert little or no effect upon the isogenic (e.g., diploid-like) cells and/or wild-type cells. In some forms, all cells used in a screen are derived from the same individual. For example in some forms, aneuploid cancer cells and non-cancer (wild-type) cells are obtained from a subject with an aneuploid cancer, and isogenic diploid-like cells are derived from the aneuploid cancer cells according to the described ReDACT methods. In other forms, the cells used in the screen are obtained from multiple different sources.

### 2. Personalized Anti-Cancer Screening

Methods for personalized cancer screening, based on the described ReDACT methods for generating isogenic diploid-like cells from aneuploid (e.g., trisomic) cells obtained from a tumor sample from a subject are described. In some forms, the methods screen trisomic cancer cells and isogenic diploid-like cell derived from the aneuploid (e.g., trisomic) cells for selective toxicity by agents that are known or unknown to have cytotoxic effects in cancer cells, to identify optimal therapeutic regimens for treating the subject.

In some forms, the methods include one or more steps including (i) selecting a subject diagnosed as having cancer; (ii) obtaining a sample of cancer cells from the subject; (iii) determining the cancer cells are aneuploid cells; (iv) providing isogenic diploid-like cells derived from the aneuploid cells according to the described ReDACT methods; (v) screening one or more active agents for selective toxicity against the aneuploid cells and the isogenic diploid-like cells; (vi) selecting an active agent having selective toxicity against the aneuploid cells; and (vii) treating the subject for the cancer by administering to the subject the selected active agent in an amount effective to treat the cancer.

### V. Methods of Treatment

Methods of treatment of cancer based on administering therapeutic agents that selectively target aneuploid cancer are described.

As demonstrated in the Examples, it has been established that chromosome 1q harbors the UCK2 gene, which encodes a pyrimidine salvage kinase that controls a rate-limiting step in the activation of certain toxic nucleotide analogs, including RX-3117 and 3-deazauridine. It has been demonstrated that the UCK2 gene product is overexpressed in human cancers that contain extra copies of chromosome 1q. As shown by the described methods for producing isogenic diploid-like cells derived from trisomic aneuploid cells, elimination of the chromosome 1q trisomy consistently decreased UCK2 protein expression in the isogenic diploid-like cell lines. Therefore, it was shown that gaining chromosome 1q imparts a collateral sensitivity to UCK2-dependent nucleotide analogs. Similarly, AHR expression is upregulated in human cancer cell lines and tumors that are trisomic for chromosome 7p. Results below show that eliminating the 7p trisomy decreases the expression of AHR and cells with a 7p trisomy were moderately but significantly more sensitive to CGS-15943 compared to 7p-disomic cells.

Thus, methods for treating aneuploid cancers are provided. The methods target one or more therapies for cancer in a subject in need thereof, including steps for (i) identifying one or more aneuploidies in cancer cells from a subject; (ii) determining aneuploid-specific targets in the aneuploid cancer cells, for example, by providing isogenic diploid-like cells derived from the aneuploid cells and identifying therapeutic agents that are selectively effective in reducing viability of the aneuploid cells; and (iii) administering one or more therapeutic agents that are selectively effective against the aneuploid-specific targets to the subject in an amount effective to reduce the viability of cancer cells in the subject. Exemplary methods employ active agents that are active on target genes and or gene products that are expressed (i.e., aberrantly expressed) by an aneuploid chromosome in an aneuploid cancer cell. In some forms, the methods administer one or more active agent(s) that is active on one or more genes that are over-expressed by an aneuploid chromosome in aneuploid cancer cells in a subject in need thereof. Therefore, in some forms, the methods for treating aneuploid cancer(s) target one or more genes that are selectively over-expressed by the aneuploid chromosome in aneuploid cancer cells in a subject. In some forms, the methods administer active agent(s) that target one or more genes that is expressed (e.g., aberrantly expressed) by an aneuploid chromosome arm that is an aneuploid of any of the 23 human chromosomes. In some forms, the methods administer active agent(s) that target one or more genes that is expressed (e.g., aberrantly expressed) by an aneuploid chromosome that is or includes the short (q) arm of any one of the 23 human chromosomes.

In some forms, the methods administer active agent(s) that target (i.e., exerts activity through) one or more genes that is expressed by an aneuploid chromosome that is or includes chromosome 1, 2, 3, 4, 5, 6, 7, 8, 9, 10, 11, 12, 13, 14, 15, 16, 17, 18, 19, 20, 21, 22, X, or Y. In some forms, the methods administer active agent(s) that target (i.e., exerts activity through) one or more genes that is expressed by an aneuploid chromosome that is or includes chromosome arm 1q, 1p, 2q, 2p, 3q, 3p, 4q, 4p, 5q, 5p, 6q, 6p, 7p, 7q, 8p, 8q, 9p, 9q, 10p, 10q, lip, 11q, 12p, 12q, 13p, 13q, 14q, 14p, 15q, 15p, 16p, 16q, 17p, 17q, 18p, 18q, 19q, 19q, 20q, 20p, or 21q, 21p, 22q, 22p, Xp, Xq, Yp, or Yq.

In particular forms, the methods administer active agent(s) that target (i.e., exerts activity through) one or more genes that is expressed by an aneuploid chromosome that is or includes chromosome 1q, 1p, 2q, 4q, 5q, 6p, 7p, 7q, 8q, 9q, 10q, 11q, 12p, 16p, 16q, 17p, 17q, 19q, 19q, or 21q. In some forms, the methods administer active agent(s) that target one or more genes expressed by an aneuploid chromosome 1q and/or aneuploid chromosome 7p and/or aneuploid chromosome 8q.

Any of the described methods can include one or more steps of selecting a subject in need of treatment. In some forms, the methods include diagnosing a subject as having cancer, or as being at increased risk of having cancer. Symptoms and physiological biomarkers associated with cancer are known in the art. In some forms, the methods include one or more steps to determine whether a subject having cancer has aneuploid cancer cells. Methods for obtaining biological samples, including samples of viable cancer cells from a subject are known in the art. In some forms, the methods further include determining the genotypic and/or phenotypic characteristics of aneuploid cancer cells derived from a subject. For example, in some forms, the methods include one or more steps to determine the genetic basis for aneuploidy in one or more aneuploid cancer cells derived from a subject having aneuploid cancer. In some forms, the subject has previously been treated for cancer. In some forms, the subject is undergoing one or more additional treatments for one or more diseases, such as cancer.

An exemplary method of treating cancer associated with aneuploidy in a subject in need thereof includes administering to the subject a therapeutic agent in an amount effective to reduce tumor burden in the subject,
whereby the cancer associated with aneuploidy over-expresses one or more genes associated with an aneuploid chromosome, and
whereby the therapeutic agent is active against one or more products of the over-expressed genes. In some forms, the aneuploid chromosome arm expresses one or more genes selected from CYP2J2, MFSD2A, UCK1, UCK2, SLC16A1, UGT1A10, DCK, SLC26A2, SLC29A1, ACTB, ABCB1, SLC39A4, ADK, FADS2, SLC35F2, PDE3A, ABCC1, SULT1A1, NQO1, SLC47A1, SLFN11, SLFN12, DCAF15, SLC6A16, SLC19A1, AHR. Exemplary therapeutic agent(s) include one or more selected from austocystin D, tunicamycin, 3-bromopyruvate, Curcumin, Gemcitabine, decitabine, bis(maltolato)oxovanadium(IV), Gemcitabine, atorvastatin, simvastatin, pitavastatin, lovastatin, fluvastatin, tepoxalin, AICAR, 6-azacytidine and 5-methylcytidine, Triciribine, ML239, YM155, DCEBIO, zardaverine, Dasatinib, Oncrasin-1, HSP90 inhibitors, Mitoxantrone, disulfiram, DCEBIO, zardaverine, Indisulam, Temsirolimus, RX-3117, 3-deazauridine, Methotrexate, and CGS-15943.

### A. Exemplary Treatment Strategies

Exemplary methods for treating aneuploid-associated cancers associated with any one or more of aneuploid chromosomes in a subject in need thereof are described.

Exemplary aneuploid cancers that can be treated according to the described methods are associated with and/or caused by the development and reproduction of aneuploid cancer cells including a specific aneuploid chromosome, such as chromosome 1, 2, 3, 4, 5, 6, 7, 8, 9, 10, 11, 12, 13, 14, 15, 16, 17, 18, 19, 20, 21, 22, X, or Y. In some forms, aneuploid cancers that can be treated according to the described methods are associated with and/or caused by the development and reproduction of aneuploid cancer cells including a specific aneuploid chromosome arm, such as chromosome arm 1q, 1p, 2q, 2p, 3q, 3p, 4q, 4p, 5q, 5p, 6q, 6p, 7p, 7q, 8p, 8q, 9p, 9q, 10p, 10q, 1p, 1q, 12p, 12q, 13p, 13q, 14q, 14p, 15q, 15p, 16p, 16q, 17p, 17q, 18p, 18q, 19q, 19q, 20q, 20p, or 21q, 21p, 22q, 22p, Xp, Xq, Yp, or Yq. In particular forms, aneuploid cancers that can be treated according to the described methods are associated with and/or caused by the development and reproduction of aneuploid cancer cells including a specific aneuploid chromosome arm, such as chromosome arm1q, 1p, 2q, 4q, 5q, 6p, 7p, 7q, 8q, 9q, 10q, 11q, 12p, 16p, 16q, 17p, 17q, 19q, 19q, or 21q.

In some forms, the described methods of treating an aneuploid cancer in a subject in need thereof include administering active agent(s) that exert activity through gene(s) expressed by any one or more of aneuploid chromosomes 1, 2, 3, 4, 5, 6, 7, 8, 9, 10, 11, 12, 13, 14, 15, 16, 17, 18, 19, 20, 21, 22, Y, or Y. In some forms, the described methods of treating an aneuploid cancer in a subject in need thereof include administering active agent(s) that exert activity through gene(s) expressed by any one or more of aneuploid chromosome arm 1q, 1p, 2q, 2p, 3q, 3p, 4q, 4p, 5q, 5p, 6q, 6p, 7p, 7q, 8p, 8q, 9p, 9q, 10p, 10q, lip, 11q, 12p, 12q, 13p, 13q, 14q, 14p, 15q, 15p, 16p, 16q, 17p, 17q, 18p, 18q, 19q, 19q, 20q, 20p, or 21q, 21p, 22q, 22p, Xp, Xq, Yp, or Yq. In particular forms, the described methods of treating an aneuploid cancer in a subject in need thereof include administering active agent(s) that exert activity through gene(s) expressed by any one or more of aneuploid chromosome arm 1q, 1p, 2q, 4q, 5q, 6p, 7p, 7q, 8q, 9q, 10q, 11q, 12p, 16p, 16q, 17p, 17q, 19q, 19q, or 21q, for example, gene(s) that are aberrantly expressed by an aneuploid cancer cell derived from the subject as compared with an isogenic diploid or diploid-like cell derived from the aneuploid cancer cell.

Certain genes are aberrantly expressed (e.g., under-expressed or over-expressed) by certain aneuploid cancer cells. Further, it is contemplated that active agents, such as therapeutic agents, having known mechanisms of activity against specific cellular pathways and processes governed by one or more of the aberrantly expressed genes can be selectively and specifically toxic to the aneuploid cancer cells that aberrantly express these genes, for example, as compared to control cells. Exemplary control cells include non-cancer cells, and/or isogenic diploid-like cells derived from the aneuploid cancer cells, for example, according to the described ReDACT methods.

### 1. Methods for Treatment Targeting 1q Aneuploid Cancers

Methods for treating chromosome 1q aneuploid-associated cancers in a subject in need thereof are provided. An exemplary aneuploid cancer is associated with and/or caused by the development and reproduction of trisomic cancer cells including an aneuploid chromosome 1q. Therefore, in some forms, methods of treating cancer in a subject in need thereof include administering active agent(s) that exert activity through gene(s) expressed by an aneuploid chromosome 1q, for example, gene(s) that are aberrantly expressed by an aneuploid cancer cell derived from the subject as compared with an isogenic diploid or diploid-like cell derived from the aneuploid cancer cell. As demonstrated in the Examples, aneuploid cancer cells having an aneuploid chromosome 1q over express pyrimidine salvage kinase. Therefore, in some forms, methods of treating an aneuploid 1q-associated cancer in a subject in need thereof include administering to the subject one or more active agent(s) that target a pyrimidine salvage kinase. An exemplary pyrimidine salvage kinase gene is the UCK2 gene and its expressed gene product, Uridine-cytidine kinase 2. Active agents that target pyrimidine salvage kinase, such as Uridine-cytidine kinase 2, and associated biochemical pathways include toxic nucleotide analogs. Exemplary toxic nucleotide analogs include RX-3117 and 3-deazauridine. Therefore, in some forms, methods of treating an aneuploid 1q-associated cancer in a subject in need thereof include administering to the subject RX-3117 and/or 3-deazauridine in an amount effective to reduce viability of aneuploid 1q cancer cells in the subject.

An exemplary method of treating cancer associated with 1q aneuploidy in a subject in need thereof includes administering to the subject a therapeutic agent that is selectively toxic to 1q aneuploid cancer cells in an amount effective to reduce tumor burden in the subject, whereby the cancer associated with aneuploidy over-expresses a pyrimidine salvage kinase, and whereby the therapeutic agent is RX-3117 and/or 3-deazauridine.

### 2. Methods for Treatment Targeting 7p Aneuploid Cancers

Methods for treating chromosome 7p aneuploid-associated cancers in a subject in need thereof are provided. An exemplary aneuploid cancer is associated with and/or caused by the development and reproduction of trisomic cancer cells including an aneuploid chromosome 7p. Therefore, in some forms, methods of treating cancer in a subject in need thereof include administering active agent(s) that exert activity through gene(s) expressed by an aneuploid chromosome 7p, for example, gene(s) that are aberrantly expressed by an aneuploid cancer cell derived from the subject as compared with an isogenic diploid or diploid-like cell derived from the aneuploid cancer cell. As demonstrated in the Examples, aneuploid cancer cells having an aneuploid chromosome 7p over express the gene AHR, which encodes a ligand-activated transcription factor. Therefore, in some forms, methods of treating an aneuploid 7p-associated cancer in a subject in need thereof include administering to the subject one or more active agent(s) that target AHR gene or the gene product thereof. Active agents that target AHR protein include CGS-15943. Therefore, in some forms, methods of treating an aneuploid 7p-associated cancer in a subject in need thereof include administering to the subject CGS-15943 in an amount effective to reduce viability of aneuploid 7p cancer cells in the subject.

An exemplary method of treating cancer associated with 7p aneuploidy in a subject in need thereof includes administering to the subject a therapeutic agent that is selectively toxic to 7p aneuploid cancer cells in an amount effective to reduce tumor burden in the subject, optionally whereby the cancer associated with aneuploidy over-expresses AHR, and optionally whereby the therapeutic agent is CGS-15943.

3. Methods for Treatment Targeting other Aneuploid Cancers

Additional pairs of trisomies and active agents, and the corresponding methods of treatment are also provided. A listing of additional aneuploid cancers, grouped according to aneuploid chromosome type, together with the implicated gene(s) that are aberrantly or selectively expressed in the aneuploid cancer cells, together with active agent(s) that are associated with a corresponding mechanism of action that is selective to the aberrantly or selectively expressed genes, is presented in Table 1, below.

TABLE 1
Aneuploidy associated genes and targeting drugs

| Trisomy | Gene | Drug |
|---|---|---|
| 1p | CYP2J2 | austocystin D |
| 1p | MFSD2A | tunicamycin |
| 1p | SLC16A1 | 3-bromopyruvate |
| 2q | UGT1A10 | Curcumin |
| 4q | DCK | Gemcitabine, decitabine |
| 5q | SLC26A2 | bis(maltolato)oxovanadium(IV) |
| 6p | SLC29A1 | Gemcitabine |
| 7p | ACTB | Statins (e.g., atorvastatin; simvastatin, |
| | | pitavastatin, lovastatin, fluvastatin) |
| 7q | ABCB1 | tepoxalin |
| 8q | SLC39A4 | AICAR |
| 9q | UCK1 | 6-azacytidine; |
| | | 5-methylcytidine |
| 10q | ADK | Triciribine |
| 11q | FADS2 | ML239 |
| 11q | SLC35F2 | YM155 |
| 12p | PDE3A | DCEBIO, zardaverine |
| 16p | ABCC1 | Dasatinib |
| 16p | SULT1A1 | Oncrasin-1 |
| 16q | NQO1 | HSP90 inhibitors |
| 17p | SLC47A1 | Mitoxantrone, disulfiram |
| 17q | SLFN11 | DNA-damaging agents |
| 17q | SLFN12 | DCEBIO, zardaverine |
| 19p | DCAF15 | Indisulam |
| 19q | SLC6A16 | Temsirolimus |
| 21q | SLC19A1 | Methotrexate; |
| | | other anti-folates |

In some forms, methods of treating an aneuploid chromosome-associated cancer in a subject in need thereof include administering to the subject one or more active agent(s) that target one or more genes that are aberrantly expressed by the aneuploid cancer cells in an amount effective to reduce the viability of the cancer cells in the subject, wherein the active agent has minimal of no toxicity on cells that lack the aneuploid chromosome. In some forms, methods for treating cancer associated with an aneuploid chromosome set forth in Table 1 in a subject in need thereof include administering to the subject one or more active agent(s) set forth in Table 1 as being associated with a target gene set forth in Table 1 in an amount effective to reduce the viability of the cancer cells in the subject.

For example, methods of treating cancer associated with 1p aneuploidy in a subject in need thereof include administering to the subject a therapeutic agent that is selectively toxic to 1p aneuploid cancer cells in an amount effective to reduce tumor burden in the subject, whereby the cancer associated with aneuploidy over-expresses CYP2J2, and the therapeutic agent includes austocystin D.

In other forms, methods of treating cancer associated with 1p aneuploidy in a subject in need thereof include administering to the subject a therapeutic agent that is selectively toxic to 1p aneuploid cancer cells in an amount effective to reduce tumor burden in the subject, whereby the cancer associated with aneuploidy over-expresses MFSD2A and whereby the therapeutic agent includes tunicamycin.

In other forms, methods of treating cancer associated with 1p aneuploidy in a subject in need thereof include administering to the subject a therapeutic agent that is selectively toxic to 1p aneuploid cancer cells in an amount effective to reduce tumor burden in the subject, whereby the cancer associated with aneuploidy over-expresses SLC16A1, and whereby the therapeutic agent includes 3-bromopyruvate.

Methods of treating cancer associated with 2q aneuploidy in a subject in need thereof include administering to the subject a therapeutic agent that is selectively toxic to 2q aneuploid cancer cells in an amount effective to reduce tumor burden in the subject, whereby the cancer associated with aneuploidy over-expresses UGT1A10 and whereby the therapeutic agent includes Curcumin.

Methods of treating cancer associated with 4q aneuploidy in a subject in need thereof include administering to the subject a therapeutic agent that is selectively toxic to 4q aneuploid cancer cells in an amount effective to reduce tumor burden in the subject, whereby the cancer associated with aneuploidy over-expresses DCK, and whereby the therapeutic agent includes Gemcitabine, and/or decitabine.

Methods of treating cancer associated with 5q aneuploidy in a subject in need thereof include administering to the subject a therapeutic agent that is selectively toxic to 5q aneuploid cancer cells in an amount effective to reduce tumor burden in the subject, whereby the cancer associated with aneuploidy over-expresses SLC26A2, and whereby the therapeutic agent includes bis(maltolato)oxovanadium(IV).

Methods of treating cancer associated with 6p aneuploidy in a subject in need thereof include administering to the subject a therapeutic agent that is selectively toxic to 6p aneuploid cancer cells in an amount effective to reduce tumor burden in the subject, whereby the cancer associated with aneuploidy over-expresses SLC29A1, and whereby the therapeutic agent includes Gemcitabine.

Methods of treating cancer associated with 7p aneuploidy in a subject in need thereof include administering to the subject a therapeutic agent that is selectively toxic to 7p aneuploid cancer cells in an amount effective to reduce tumor burden in the subject, whereby the cancer associated with aneuploidy over-expresses ACTB, and whereby the therapeutic agent includes a statin, such as atorvastatin, and/or simvastatin and/or pitavastatin, and/or lovastatin, and/or fluvastatin.

Methods of treating cancer associated with 7q aneuploidy in a subject in need thereof include administering to the subject a therapeutic agent that is selectively toxic to 7q aneuploid cancer cells in an amount effective to reduce tumor burden in the subject, whereby the cancer associated with aneuploidy over-expresses ABCB1, and whereby the therapeutic agent includes tepoxalin.

Methods of treating cancer associated with 8q aneuploidy in a subject in need thereof include administering to the subject a therapeutic agent that is selectively toxic to 8q aneuploid cancer cells in an amount effective to reduce tumor burden in the subject, whereby the cancer associated with aneuploidy over-expresses SLC39A4, and whereby the therapeutic agent includes AICAR.

Methods of treating cancer associated with 9q aneuploidy in a subject in need thereof include administering to the subject a therapeutic agent that is selectively toxic to 9q aneuploid cancer cells in an amount effective to reduce tumor burden in the subject, whereby the cancer associated with aneuploidy over-expresses UCK1, and whereby the therapeutic agent includes 6-azacytidine and/or 5-methylcytidine.

Methods of treating cancer associated with 10q aneuploidy in a subject in need thereof include administering to the subject a therapeutic agent that is selectively toxic to 10q aneuploid cancer cells in an amount effective to reduce tumor burden in the subject, whereby the cancer associated with aneuploidy over-expresses ADK, and whereby the therapeutic agent includes Triciribine.

Methods of treating cancer associated with 11q aneuploidy in a subject in need thereof include administering to the subject a therapeutic agent that is selectively toxic to 11q aneuploid cancer cells in an amount effective to reduce tumor burden in the subject, whereby the cancer associated with aneuploidy over-expresses FADS2, and whereby the therapeutic agent includes ML239.

Methods of treating cancer associated with 11q aneuploidy in a subject in need thereof include administering to the subject a therapeutic agent that is selectively toxic to 11q aneuploid cancer cells in an amount effective to reduce tumor burden in the subject, whereby the cancer associated with aneuploidy over-expresses SLC35F2, and whereby the therapeutic agent includes YM155.

Methods of treating cancer associated with 12p aneuploidy in a subject in need thereof include administering to the subject a therapeutic agent that is selectively toxic to 12q aneuploid cancer cells in an amount effective to reduce tumor burden in the subject, whereby the cancer associated with aneuploidy over-expresses 12p, whereby the one or more over-expressed genes includes PDE3A and, whereby the therapeutic agent includes DCEBIO, and/or zardaverine.

Methods of treating cancer associated with 16p aneuploidy in a subject in need thereof include administering to the subject a therapeutic agent that is selectively toxic to 16p aneuploid cancer cells in an amount effective to reduce tumor burden in the subject, whereby the cancer associated with aneuploidy over-expresses ABCC1, whereby the therapeutic agent includes Dasatinib.

Methods of treating cancer associated with 16p aneuploidy in a subject in need thereof include administering to the subject a therapeutic agent that is selectively toxic to 16p aneuploid cancer cells in an amount effective to reduce tumor burden in the subject, whereby the cancer associated with aneuploidy over-expresses SULT1A1, whereby the therapeutic agent includes Oncrasin-1.

Methods of treating cancer associated with 16q aneuploidy in a subject in need thereof include administering to the subject a therapeutic agent that is selectively toxic to 16q aneuploid cancer cells in an amount effective to reduce tumor burden in the subject, whereby the cancer associated with aneuploidy over-expresses NQO1, and whereby the therapeutic agent includes an HSP90 inhibitor.

Methods of treating cancer associated with 17p aneuploidy in a subject in need thereof include administering to the subject a therapeutic agent that is selectively toxic to 17p aneuploid cancer cells in an amount effective to reduce tumor burden in the subject, whereby the cancer associated with aneuploidy over-expresses SLC47A1, and whereby the therapeutic agent includes Mitoxantrone and/or disulfiram.

Methods of treating cancer associated with 17q aneuploidy in a subject in need thereof include administering to the subject a therapeutic agent that is selectively toxic to 17q aneuploid cancer cells in an amount effective to reduce tumor burden in the subject, whereby the cancer associated with aneuploidy over-expresses SLFN11 and whereby the therapeutic agent(s) include DNA-damaging agent(s).

Methods of treating cancer associated with 17q aneuploidy in a subject in need thereof include administering to the subject a therapeutic agent that is selectively toxic to 17q aneuploid cancer cells in an amount effective to reduce tumor burden in the subject, whereby the cancer associated with aneuploidy over-expresses SLFN12, and whereby the therapeutic agent includes DCEBIO and/or zardaverine.

Methods of treating cancer associated with 19p aneuploidy in a subject in need thereof include administering to the subject a therapeutic agent that is selectively toxic to 19p aneuploid cancer cells in an amount effective to reduce tumor burden in the subject, whereby the cancer associated with aneuploidy over-expresses includes DCAF15, whereby the therapeutic agent includes Indisulam.

Methods of treating cancer associated with 19q aneuploidy in a subject in need thereof include administering to the subject a therapeutic agent that is selectively toxic to 19q aneuploid cancer cells in an amount effective to reduce tumor burden in the subject, whereby the cancer associated with aneuploidy over-expresses SLC6A16, and whereby the therapeutic agent includes Temsirolimus.

Methods of treating cancer associated with 21q aneuploidy in a subject in need thereof include administering to the subject a therapeutic agent that is selectively toxic to 21q aneuploid cancer cells in an amount effective to reduce tumor burden in the subject, whereby the cancer associated with aneuploidy over-expresses SLC19A1, and whereby the therapeutic agent includes an anti-folate, such as Methotrexate.

### B. Personalized Anti-Cancer Treatment Targeting Aneuploid Cancers

Methods for treating a specific genotype of aneuploid-associated cancer in a subject in need thereof are provided, including one or more steps to identify the aneuploid-specific sensitivity of aneuploid cancer cells derived from the subject. Therefore, methods for the “personalized” selection and administration of a therapeutic agent(s) to a subject having an aneuploid cancer are described, including one or more steps to identify the genotype and/or phenotype of an aneuploid cancer cell derived from the subject and/or to screen for aneuploid-specific toxicity of one or more active agents. Typically, the methods screen aneuploid cancer cells for aneuploid-specific toxicity according to the described in vitro ReDACT methods.

In preferred forms, one or more active agents that are toxic to and/or reduce the viability of the aneuploid cancer cells in a subject having an aneuploid cancer are less toxic or non-toxic to non-aneuploid cells in the subject. In some forms, the one or more active agents that are toxic to and/or reduce the viability of the aneuploid cancer cells in a subject having an aneuploid cancer have little or no effect upon the viability of diploid cells in the subject. Therefore, in some forms, the methods provide therapeutic regimens for the treatment of aneuploid-specific cancer in a subject in need thereof with minimal or no toxic side-effects for non-cancer cells in the subject. For example, in some forms, the methods include one or more steps to screen autologous aneuploid cancer cells derived from a subject with an aneuploid cancer to determine efficacy of one or more active agents to selectively reduce the viability and/or kill the aneuploid cancer cells as compared to isogenic diploid-like cells derived from the aneuploid cancer cells, according to the described ReDACT methods. An exemplary method includes one or more steps, including: (i) screening of aneuploid cancer cells derived from a subject for susceptibility to an active agent; (ii) selecting an active agent as selectively toxic to the aneuploid cancer cell when the agent reduces the viability of the aneuploid cancer cell, optionally whereby the reduction in viability of the aneuploid cancer cell is greater than the reduction in viability of the isogenic control cell contacted by the same active agent(s) under the same conditions; and (iii) administering to the subject the therapeutic agent that is selectively toxic to the aneuploid cancer cells in an amount effective to reduce tumor burden in the subject.

### C. Diseases to be Treated

Methods of treating a subject having a disease or disorder associated with aneuploidy are provided. Typically, the methods treat cancer associated with aneuploidy in a subject in need thereof.

### 1. Cancer

In some forms, the methods treat or prevent cancer associated with aneuploidy in a subject. Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell., 144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

Methods of treating a subject having cancer, including administering to the subject an effective amount of a pharmaceutical composition including an anticancer therapy including, but not limited to, those discussed herein.

The disclosed compositions and methods of treatment thereof are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth.

The types of cancer that can be treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some forms, the compositions are used to treat multiple cancer types concurrently. The compositions can also be used to treat metastases or tumors at multiple locations.

The cancer can be of any type, including but not limited to leukemias including, but not limited to acute lymphoblastic leukemia, acute myeloid leukemia, myelodysplastic syndromes, chronic lymphocytic leukemia, chronic myelogenous leukemia, hairy cell leukemia; polycythemia vera; lymphomas including, but not limited to, Burkitt's lymphoma, follicular lymphoma, Hodgkin's lymphoma, non-Hodgkin's lymphoma; multiple myelomas including, but not limited to, monoclonal gammopathy of undetermined significance (MGUS), isolated plasmacytoma, extramedullary plasmacytoma, smoldering multiple myeloma, Waldenstrom's macroglobulinemia; bone cancers including, but not limited to, chondrosarcoma, chordoma, Ewing's sarcoma, osteosarcoma, giant cell tumor of bone, fibrosarcoma; soft-tissue sarcomas including, but not limited to, angiosarcoma (hemangiosarcoma), desmoplastic small round cell tumors, Kaposi's sarcoma, gastrointestinal stromal tumor, leiomyosarcoma, liposarcoma, lymphangiosarcoma, myxosarcoma, rhabdomyosarcoma, synovial sarcoma; brain tumors and nervous system cancers including, but not limited to, astrocytoma, chordoma, gliomas, ependymomas, glioblastoma, gliomas, medulloblastoma, malignant peripheral nerve sheath tumors, neuroblastoma, optic nerve glioma, pineoblastoma, primary brain lymphoma, primitive neuroectodermal tumor; breast cancers including, but not limited to, inflammatory breast cancer, invasive ductal carcinoma, invasive lobular carcinoma, medullary breast cancer, mucinous breast cancer, Paget's disease, Phyllode's tumor, tubular breast cancer; adrenal cancer including, but not limited to, adrenocortical carcinoma, pheochromocytoma; thyroid cancer including, but not limited to, anaplastic thyroid cancer, follicular thyroid cancer, Hurthle cell thyroid cancer, medullary thyroid cancer, papillary thyroid cancer; parathyroid cancer including, but not limited to, parathyroid carcinoma; pancreatic cancer including, but not limited to, adenocarcinoma, acinar cell carcinoma, pancreatoblastoma, pancreatic neuroendocrine neoplasms; ocular cancers including, but not limited to, ocular melanoma, retinoblastoma; vaginal cancers including but not limited to, vaginal adenocarcinoma, clear cell adenocarcinoma, squamous cell carcinoma, germ cell tumors, rhabdomyosarcoma, vaginal melanoma; vulvar cancer including but not limited to, adenocarcinoma, Bartholin gland carcinoma, basal cell carcinoma, squamous cell carcinoma; cervical cancers including, but not limited to, adenocarcinoma, squamous cell carcinoma; endometrial cancers including, but not limited to, endometrioid adenocarcinoma, serous carcinoma, clear cell carcinoma, mucinous carcinoma, mixed or undifferentiated carcinoma, endometrial stromal sarcoma; uterine cancers including, but not limited to, uterine sarcoma; ovarian cancers including, but not limited to, epithelial ovarian carcinomas, primary peritoneal carcinoma, fallopian tube cancer, germ cell tumors, stromal tumors, ovarian sarcoma, borderline tumor; esophageal cancers including, but not limited to, squamous-cell carcinoma, adenocarcinoma, small cell carcinoma; gastric cancers including, but not limited to, adenocarcinoma, lymphoma, gastrointestinal stromal tumors, neuroendocrine neoplasms, hereditary diffuse gastric cancer, familial intestinal gastric cancer; small bowel cancer including, but not limited to, adenocarcinoma, neuroendocrine neoplasm, lymphoma, gastrointestinal stromal tumor; appendiceal cancers including, but not limited to, adenocarcinoma, neuroendocrine neoplasms; colon cancers including, but not limited to, adenocarcinoma, neuroendocrine neoplasm, lymphoma, squamous cell carcinoma; rectal cancers including, but not limited to, adenocarcinoma, neuroendocrine neoplasm, lymphoma; anal cancers including, but not limited to, squamous cell carcinoma, adenocarcinoma, small cell carcinoma, melanoma; liver cancers including, but not limited to, hepatocellular carcinoma, angiosarcoma, hemangiosarcoma, and hepatoblastoma; gallbladder cancers including, but not limited to, adenocarcinoma, squamous cell carcinoma; cholangiocarcinoma including, but not limited to, intrahepatic cholangiocarcinoma, perihilar cholangiocarcinoma, distal cholangiocarcinoma; lung and pleural cancers including but not limited to, non-small cell lung cancer, squamous cell carcinoma, adenocarcinoma, large-cell carcinoma, small-cell lung cancer, mesothelioma; testicular cancers including, but not limited to, germ cell tumor, sex-cord stromal tumor, lymphoma; prostate cancers including, but not limited to, adenocarcinoma, squamous cell carcinoma, transitional cell carcinoma, neuroendocrine neoplasm, sarcoma; penile cancer including, but not limited to, squamous cell carcinoma, melanoma, basal cell carcinoma, adenocarcinoma, sarcoma; kidney or ureteral cancer including, but not limited to, renal cell carcinoma, transitional cell cancer, squamous cell carcinoma, adenocarcinoma, sarcoma, lymphoma, Wilms tumor, neuroendocrine neoplasm, carcinosarcoma; bladder cancer including, but not limited to, transitional cell carcinoma, squamous cell carcinoma, adenocarcinoma, carcinosarcoma, small cell carcinoma; oropharyngeal cancers including, but not limited to, squamous cell carcinoma, verrucous carcinoma; basal cancers, melanoma; salivary gland cancers including, but not limited to, adenocarcinoma, mucoepidermoid carcinoma, and adenoidcystic carcinoma; skin cancers including, but not limited to, basal cell carcinoma, squamous cell carcinoma, Merkel cell carcinoma, and melanoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Exemplary 1q aneuploid-associated cancers that can be treated according to the described methods include, but are not limited to ovarian cancer, prostate cancer, acute myeloid leukemia, endometrial carcinoma, gastric cancer, melanoma pancreatic cancer, non-small cell lung cancer, Adrenocortical carcinoma, sarcoma, renal cancer, breast cancer and colorectal cancer.

### 2. Other Disease or Disorders

In some forms the methods treat one or more non-cancer disease or disorder associated with aneuploidy in a subject in need thereof. For example, in some forms the methods treat one or more genetic disease or disorders in a subject, such as a hereditary genetic disease or disorder, or a somatic genetic disease or disorder that is associated with aneuploidy in a subject.

### D. Effective Amounts

The effective amount or therapeutically effective amount of an active agent, and/or a pharmaceutical composition thereof for use in the described methods for treating a disease or disorder associated with aneuploidy in a subject can be a dosage sufficient to treat, inhibit, or alleviate one or more symptoms of a disease or disorder associated with aneuploidy, such as a cancer or proliferative disease, or to otherwise provide a desired pharmacologic and/or physiologic effect, for example, reducing, inhibiting, or reversing one or more of the underlying pathophysiological mechanisms underlying a disease or disorder associated with aneuploidy, such as cancer.

In some forms, when administration of an active agent, and/or a pharmaceutical composition thereof elicits an anti-cancer response, the amount administered can be expressed as the amount effective to achieve a desired anti-cancer effect in the recipient. For example, in some forms, the amount of the active agent, and/or a pharmaceutical composition thereof is effective to inhibit the viability or proliferation of cancer cells, such as aneuploid cancer cells in the recipient. In some forms, the amount of the active agent, and/or a pharmaceutical composition thereof is effective to reduce the tumor burden in the recipient, or reduce the total number of cancer cells, and combinations thereof. In other forms, the amount of the active agent, and/or a pharmaceutical composition thereof is effective to reduce one or more symptoms or signs of cancer in a cancer patient, or signs of disease in a patient having a disease or disorder.

The effective amount of the active agent, and/or a pharmaceutical composition thereof that is required will vary from subject to subject, depending on the species, age, weight and general condition of the subject, the severity of the disorder being treated, and its mode of administration. Thus, it is not possible to specify an exact amount for every active agent, and/or a pharmaceutical composition thereof. However, an appropriate amount can be determined by one of ordinary skill in the art using only routine experimentation given the teachings herein. For example, effective dosages and schedules for administering the active agent, and/or a pharmaceutical composition thereof can be determined empirically, and making such determinations is within the skill in the art. In some forms, the dosage ranges for the administration of the active agent, and/or a pharmaceutical composition thereof are those large enough to effect reduction in cancer cell proliferation or viability, or to reduce tumor burden for example.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the active agent, and/or a pharmaceutical composition thereof used for treatment can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions and can generally be estimated based on EC₅₀s found to be effective in in vitro and in vivo animal models.

The infusion can be repeated as often and as many times as the patient can tolerate until the desired response is achieved. Compositions can also be administered once or multiple times at these dosages. The optimal dosage and treatment regime for a particular patient can readily be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intra-tumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions including modified cells, such as therapeutic T cells, according to the methods described herein can be determined by evaluating the aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks, or months) following treatment.

In exemplary forms, one or more active agents are formulated in a pharmaceutically acceptable excipient for administration in vivo, and the amount of each active agent administered is from about 0.001 mg/kg body weight of the subject to about 100 mg/kg body weight of the subject, inclusive.

### E. Modes of Administration

Any active agent, and/or a pharmaceutical composition thereof can be used therapeutically in combination with a pharmaceutically acceptable carrier. The active agents and/or compositions described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions described herein can include, but are not limited to, carriers, thickeners, diluents, buffers, preservatives, surface active agents and the like in addition to the therapeutic(s) of choice.

Pharmaceutical compositions and optionally one or more additional therapeutic agents can be administered to the subject in a number of ways depending on whether local or systemic treatment is desired, and on the area to be treated. Thus, for example, a pharmaceutical composition can be administered as an intravenous infusion, or directly injected into a specific site, for example, into or surrounding a tumor. Moreover, a pharmaceutical composition can be administered to a subject as an ophthalmic solution and/or ointment to the surface of the eye, vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. In some forms, the compositions are administered directly into a tumor or tissue, e.g., stereotactically.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant including a porous, non-porous, or gelatinous material).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Administration of the pharmaceutical compositions can be localized (i.e., to a particular region, physiological system, tissue, organ, or cell type) or systemic.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### F. Combination Therapy

Combination treatments, including the described aneuploid-specific therapeutic regimens, together with one or more additional therapeutic agents and/or procedures are provided.

In some forms, one or more therapeutic agent is administered to the subject in combination with one or more additional therapeutic agents. For example, in some forms, the combination of two or more active agents reduces the viability of aneuploid cancer cells in vivo to a greater extent than the equivalent amount of any one of the active agents when administered alone to the subject. In some forms, the combination of the two or more active agents reduces the viability of aneuploid cancer cells in vivo to a greater extent than the additive amount of the same active agents when administered alone to the subject. In some forms, the combination of the two or more active agents reduces the viability of aneuploid cancer cells in vivo at a lower dosage than the dosage required for an equivalent reduction in viability of aneuploid cancer cells in vivo when the same active agents are administered alone to the subject. Preferably, the combination of the two or more active agents has no or minimal toxicity to non-aneuploid cells in vivo.

Any of the disclosed aneuploid cancer-specific active agent(s), and/or pharmaceutical composition(s) thereof can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one agent is given first followed by the second).

Examples of preferred additional therapeutic agents include other conventional therapies known in the art for treating the desired disease, disorder or condition. In some forms, the therapeutic agent is one or more other targeted therapies (e.g., a targeted cancer therapy) and/or immune-checkpoint blockage agents (e.g., anti-CTLA-4, anti-PD1, and/or anti-PDL1 agents such as antibodies).

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

### 1. Additional Anti-Cancer Agents

In some forms, the methods administer compositions in combination with one or more additional anti-cancer agents to a subject.

In the context of cancer, targeted therapies are therapeutic agents that block the growth and spread of cancer by interfering with specific molecules (“molecular targets”) that are involved in the growth, progression, and spread of cancer. Many different targeted therapies have been approved for use in cancer treatment.

These therapies include hormone therapies, signal transduction inhibitors, apoptosis inducers, angiogenesis inhibitors, immunotherapies, and toxin delivery molecules. Numerous antineoplastic drugs can be used in combination with the disclosed pharmaceutical compositions. In some forms, the additional therapeutic agent is a chemotherapeutic or antineoplastic drug. The majority of chemotherapeutic drugs can be divided into alkylating agents, antimetabolites, anthracyclines, plant alkaloids, topoisomerase inhibitors, monoclonal antibodies, and other anti-tumor agents.

### VI. Kits

The described compositions, reagents, and other materials for cellular genomic engineering can be packaged together in any suitable combination as a kit useful for performing, or aiding in the performance of, the methods. It is useful if the components in a kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

The disclosed compositions and methods can be further understood through the following examples.

The disclosed invention can be further illustrated by the following numbered paragraphs:
1. A method for creating a viable isogenic cell or tissue having an ablated chromosome or a fragment thereof from a precursor cell or tissue, including:
(i) introducing into a precursor cell or tissue a gene editing composition configured to cleave a target chromosome or target chromosome arm and optionally a nucleic acid configured to integrate into the target chromosome or target chromosome arm to form an isogenic cell or tissue including a cleaved target chromosome or cleaved target chromosome arm;
   and
(ii) optionally isolating the isogenic cell or tissue,
   optionally wherein the cleaved target chromosome or target chromosome arm is
      lost from the cell or tissue.
2. The method of paragraph 1, wherein the gene editing composition is selected from the group consisting of Clustered Regularly Interspaced Short Palindromic Repeats/CRISPR associated protein (CRISPR/Cas), zinc finger nucleases (ZFN), and transcription activator-like effector nucleases (TALEN).
3. The method of paragraph 1 or 2, wherein the gene editing composition includes a CRISPR/Cas composition.
4. The method of any one of paragraphs 1-3, wherein the gene editing composition includes a CRISPR/Cas composition including a guide RNA (gRNA) configured to cleave nucleotides proximal to a centromere in the target chromosome arm.
5. The method of paragraph 4, wherein the gene editing composition includes a CRISPR/Cas composition,
wherein the nucleic acid configured to integrate into the target chromosome arm includes a reporter gene,
wherein the CRISPR/Cas composition is configured to cleave the target chromosome arm between the integrated reporter gene and a centromere of the target chromosome arm; and
wherein isolating the isogenic cell or tissue includes selecting a cell or tissue lacking the reporter gene.
6. The method of paragraph 5, wherein the reporter gene is selected from the group consisting of herpesvirus thymidine kinase (HSV-TK), Cytosine Deaminase, Diphtheria Toxin, and a fluorescent protein.
7. The method of paragraph 6, wherein the reporter gene is herpesvirus thymidine kinase (HSV-TK); and
wherein the isolating in step (ii) includes contacting the cell or tissue with ganciclovir, and isolating an isogenic cell or tissue that is not susceptible to ganciclovir.
8. The method of any one of paragraphs 1-3, wherein the gene editing composition includes a CRISPR/Cas composition,
wherein the nucleic acid configured to integrate into the target chromosome arm includes a telomere repeat sequence,
wherein the CRISPR/Cas composition is configured to cleave nucleotides proximal to a centromere in the target chromosome arm and integrate the telomere repeat sequence to provide a de novo telomere; and
wherein isolating the isogenic cells or tissue includes selecting cells or tissue including the de novo telomere.
9. The method of paragraph 8, wherein the telomere repeat sequence includes ˜100 repeats of the human telomere seed sequence, and
optionally a reporter gene.
10. The method of paragraph 8 or 9, wherein the telomere repeat sequence includes a reporter gene, and wherein isolating the isogenic cell or tissue includes selecting a cell or tissue expressing the reporter gene.
11. The method of any one of paragraphs 1 to 10, wherein the cell or tissue is a human cell or tissue, and
wherein the target chromosome is selected from the group consisting of chromosome 1, 2, 3, 4, 5, 6, 7, 8, 9, 10, 11, 12, 13, 14, 15, 16, 17, 18, 19, 20, 21, 22, X, or Y;
or
wherein the target chromosome arm is selected from the group consisting of arm 1q, 1p, 2q, 2p, 3q, 3p, 4q, 4p, 5q, 5p, 6q, 6p, 7p, 7q, 8p, 8q, 9p, 9q, 10p, 10q, lip, 11q, 12p, 12q, 13p, 13q, 14q, 14p, 15q, 15p, 16p, 16q, 17p, 17q, 18p, 18q, 19q, 19q, 20q, 20p, or 21q, 21p, 22q, 22p, Xp, Xq, Yp, or Yq.
12. The method of any one of paragraphs 1 to 11, wherein the target chromosome arm is selected from the group consisting of chromosome 1q, chromosome 7p, and chromosome 8q,
optionally wherein the target chromosome arm is chromosome 1q.
13. The method of any one of paragraphs 1 to 12, wherein the target chromosome or target chromosome arm expresses one or more genes selected from the group consisting of CYP2J2, MFSD2A, UCK1, UCK2, SLC16A1, UGT1A10, DCK, SLC26A2, SLC29A1, ACTB, ABCB1, SLC39A4, ADK, FADS2, SLC35F2, PDE3A, ABCC1, SULT1A1, NQO1, SLC47A1, SLFN11, SLFN12, DCAF15, SLC6A16, SLC19A1, and AHR.
14. The method of any one of paragraphs 1 to 13, wherein the precursor cell or tissue is an aneuploid cell or tissue,
optionally wherein the target chromosome or target chromosome arm is an aneuploid chromosome or chromosome arm.
15. The method of any one of paragraphs 1 to 14, wherein the precursor cell or tissue is a cancer cell or cancer tissue,
optionally wherein the cancer cell or tissue is an aneuploid cancer cell or an aneuploid cancer tissue.
16. The method of paragraph 15, wherein the aneuploid cancer cell or an aneuploid cancer tissue is derived from a subject with cancer.
17. The method of paragraph 16, further including prior to step (i), obtaining the aneuploid cancer cell or tissue from the subject.
18. The method of any one of paragraphs 16 to 17, wherein the cancer is selected from the group consisting of anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer, uterine cancer, acute myeloid leukemia, and adrenocortical carcinoma.
19. An isogenic cell or tissue formed from a precursor cell or tissue by the method of any one of paragraphs 1 to 18.
20. A method for screening of one or more candidate active agent(s) for selective cytotoxicity to aneuploid cancer cells or aneuploid cancer tissue, including:
(a) contacting a viable aneuploid cancer cell or aneuploid cancer tissue with one or more active agents under conditions suitable for cell or tissue culture,
wherein the aneuploid cancer cell or aneuploid cancer tissue includes an additional chromosome or chromosome arm relative to an isogenic control cell or tissue, and
(b) selecting the one or more candidate active agent(s) as selectively toxic to the aneuploid cancer cell or aneuploid cancer tissue when the one or more candidate active agent(s) reduces the viability of the aneuploid cancer cell or aneuploid cancer tissue to a greater extent than the reduction in viability of the isogenic control cell or tissue contacted by the same candidate active agent(s) under the same conditions.
21. The method of paragraph 20, wherein the isogenic control cell or tissue that lacks the additional chromosome or arm is derived from the aneuploid cancer cell or tissue by selective inactivation of the aneuploid chromosome or arm.
22. The method of paragraph 21, wherein the isogenic control cell or tissue is derived from the aneuploid cancer cell or aneuploid cancer tissue according to a method including of any one of paragraphs 1-18.
23. The method of any one of paragraphs 20 to 22, wherein the one or more candidate active agent(s) inhibit, reduce or otherwise interfere with the one or more differently expressed genes and/or expressed gene products.
24. The method of any one of paragraphs 20-23, wherein the viable aneuploid cancer cell or aneuploid cancer tissue includes one or more differently expressed gene(s) and/or expressed gene product(s) relative to the isogenic control cell or tissue.
25. The method of paragraph 24, wherein the one or more differently expressed genes and/or expressed gene products include importer pumps and/or enzymes that are active on the one or more candidate active agent(s).
26. The method of paragraph 25, wherein the enzyme modifies the structure of the one or more candidate active agent(s) from a first form to a second form,
optionally wherein the second form is more toxic to the aneuploid cancer cell or aneuploid cancer tissue relative to the first form.
27. The method of any one of paragraphs 20 to 26, wherein the active agent(s) is one or more selected from the group consisting of austocystin D, tunicamycin, 3-bromopyruvate, Curcumin, Gemcitabine, decitabine, bis(maltolato)oxovanadium(IV), Gemcitabine, atorvastatin, simvastatin, pitavastatin, lovastatin, fluvastatin, tepoxalin, AICAR, 6-azacytidine and 5-methylcytidine, Triciribine, ML239, YM155, DCEBIO, zardaverine, Dasatinib, Oncrasin-1, HSP90 inhibitors, Mitoxantrone, disulfiram, DCEBIO, zardaverine, Indisulam, Temsirolimus, RX-3117, 3-deazauridine, Methotrexate, and CGS-15943.
28. The method of any one of paragraphs 20 to 27, wherein the aneuploid cancer cells or aneuploid cancer tissue are autologous cells or tissue derived from a subject having cancer.
29. The method of paragraph 28 wherein the cancer is selected from the group consisting of anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer and uterine cancer, acute myeloid leukemia, and adrenocortical carcinoma.
30. The method of paragraph 28 or 29, further including one or more steps of treating the subject for cancer by administering the selected one or more selectively toxic candidate active agent(s) to the subject in an amount effective to reduce or prevent one or more symptoms of the cancer in the subject.
31. The method of any one of paragraphs 28-30, further including, prior to step (a), one or more steps to identify the subject having cancer.
32. The method of any one of paragraphs 20-31, further including, prior to step (a), one or more steps to identify the cancer as an aneuploid cancer.
33. A method of treating an aneuploid cancer in a subject in need thereof, including administering to the subject a therapeutic agent in an amount effective to reduce or prevent one or more symptoms of the aneuploid cancer in the subject,
wherein the therapeutic agent reduces the viability of aneuploid cancer cells more than the viability of isogenic control cells.
34. The method of paragraph 33, wherein the isogenic control cells are created from the aneuploid cancer cells according to the method of any one of paragraphs 1-18.
35. The method of paragraph 33 or 34, wherein the therapeutic agent is administered to the subject in an amount effective to reduce the viability of aneuploid cancer cells in the subject,
wherein the aneuploid cancer cells aberrantly expresses one or more genes associated with an aneuploid chromosome or arm, and
wherein the therapeutic agent is active against the one or more aberrantly-expressed genes, or the expression products thereof.
36. The method of paragraph 34, wherein the cancer is selected from the group consisting of anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer and uterine cancer, acute myeloid leukemia, and adrenocortical carcinoma.
37. The method of any one of paragraphs 33 to 36, wherein the aneuploid chromosome is selected from the group consisting of chromosome 1, 2, 3, 4, 5, 6, 7, 8, 9, 10, 11, 12, 13, 14, 15, 16, 17, 18, 19, 20, 21, 22, X, or Y;
or
wherein the aneuploid chromosome arm is selected from the group consisting of arm 1q, 1p, 2q, 2p, 3q, 3p, 4q, 4p, 5q, 5p, 6q, 6p, 7p, 7q, 8p, 8q, 9p, 9q, 10p, 10q, lip, 11q, 12p, 12q, 13p, 13q, 14q, 14p, 15q, 15p, 16p, 16q, 17p, 17q, 18p, 18q, 19q, 19q, 20q, 20p, or 21q, 21p, 22q, 22p, Xp, Xq, Yp, or Yq.
38. The method of any one of paragraphs 33 to 37, wherein the aneuploid chromosome or arm expresses one or more genes selected from the group consisting of CYP2J2, MFSD2A, UCK1, UCK2, SLC16A1, UGT1A10, DCK, SLC26A2, SLC29A1, ACTB, ABCB1, SLC39A4, ADK, FADS2, SLC35F2, PDE3A, ABCC1, SULT1A1, NQO1, SLC47A1, SLFN11, SLFN12, DCAF15, SLC6A16 and SLC19A1.
39. The method of any one of paragraphs 33 to 37, wherein the therapeutic agent(s) is one or more selected from the group consisting of austocystin D, tunicamycin, 3-bromopyruvate, Curcumin, Gemcitabine, decitabine, bis(maltolato)oxovanadium(IV), Gemcitabine, atorvastatin, simvastatin, pitavastatin, lovastatin, fluvastatin, tepoxalin, AICAR, 6-azacytidine and 5-methylcytidine, Triciribine, ML239, YM155, DCEBIO, zardaverine, Dasatinib, Oncrasin-1, HSP90 inhibitors, Mitoxantrone, disulfiram, DCEBIO, zardaverine, Indisulam, Temsirolimus, RX-3117, 3-deazauridine, Methotrexate, and CGS-15943.
40. The method of any one of paragraphs 33 to 39, wherein the therapeutic agent is administered to the subject in combination with one or more additional therapeutic agents.
41. The method of paragraph 40, wherein the combination of the two or more active agents reduces the viability of aneuploid cancer cells in vivo to a greater extent than the equivalent amount of any one of the active agents when administered alone to the subject.
42. The method of paragraph 40 or 41, wherein the combination of the two or more active agents reduces the viability of aneuploid cancer cells in vivo to a greater extent than the additive amount of the same active agents when administered alone to the subject.
43. The method of any one of paragraphs 40 to 42, wherein the combination of the two or more active agents reduces the viability of aneuploid cancer cells in vivo at a lower dosage than the dosage required for an equivalent reduction in viability of aneuploid cancer cells in vivo when the same active agents are administered alone to the subject.
44. The method of any one of paragraphs 40 to 43, wherein the combination of the two or more active agents has no or minimal toxicity to non-aneuploid cells in vivo.
45. The method of any one of paragraphs 33-44, wherein the one or more active agents are formulated in a pharmaceutically acceptable excipient for administration in vivo, and
wherein the amount of each active agent administered is from about 0.001 mg/kg body weight of the subject to about 100 mg/kg body weight of the subject, inclusive.
46. The method of any one of paragraphs 33-45, wherein the aneuploid chromosome arm is 1q, 7p, or 8q.
47. The method of paragraph 46, wherein the aneuploid chromosome arm is 1q or 7p.
48. The method of paragraph 47, wherein
(a) the aneuploid chromosome arm is 1q and the one or more over-expressed genes include Uridine-Cytidine Kinase 2 (UCK2), or
(b) the aneuploid chromosome is 7p and the one or more over-expressed genes include AHR.
49. The method of paragraph 48, including
(a) wherein the therapeutic agent includes a UCK2-activated chemotherapy drug optionally selected from RX-3117 and/or 3-deazauridine.
50. The method of paragraph 48, including
(b) wherein the therapeutic agent includes a compound that binds to AHR and causes it to upregulate the expression of pro-apoptotic genes and optionally is CGS-15943.
51. The method of paragraph 47, wherein the one or more over-expressed genes includes CYP2J2.
52. The method of paragraph 51, wherein the therapeutic agent includes austocystin D.
53. The method of paragraph 47, wherein the one or more over-expressed genes includes MFSD2A.
54. The method of paragraph 53, wherein the therapeutic agent includes tunicamycin.
55. The method of paragraph 47, wherein the one or more over-expressed genes includes SLC16A1.
56. The method of paragraph 55, wherein the therapeutic agent includes 3-bromopyruvate.
57. The method of any one of paragraphs 33-45, wherein the aneuploid chromosome is 2q, and
wherein the one or more over-expressed genes includes UGT1A10.
58. The method of paragraph 57, wherein the therapeutic agent includes Curcumin.
59. The method of any one of paragraphs 33-45, wherein the aneuploid chromosome is 4q, and
wherein the one or more over-expressed genes includes DCK.
60. The method of paragraph 59, wherein the therapeutic agent includes Gemcitabine, and/or decitabine.
61. The method of any one of paragraphs 33-45, wherein the aneuploid chromosome is 5q, and
wherein the one or more over-expressed genes includes SLC26A2.
62. The method of paragraph 61, wherein the therapeutic agent includes bis(maltolato)oxovanadium(IV).
63. The method of any one of paragraphs 33-45, wherein the aneuploid chromosome is 6p, and
wherein the one or more over-expressed genes includes SLC29A1.
64. The method of paragraph 63, wherein the therapeutic agent includes Gemcitabine.
65. The method of any one of paragraphs 33-45, wherein the aneuploid chromosome is 7p or 7q.
66. The method of paragraph 65, wherein the one or more over-expressed genes includes ACTB.
67. The method of paragraph 66, wherein the therapeutic agent includes a statin.
68. The method of paragraph 67, wherein the statin includes atorvastatin, and/or simvastatin and/or pitavastatin, and/or lovastatin, and/or fluvastatin.
69. The method of paragraph 65, wherein the one or more over-expressed genes includes ABCB1.
70. The method of paragraph 69, wherein the therapeutic agent includes tepoxalin.
71. The method of any one of paragraphs 33-45, wherein the aneuploid chromosome is 8q, and
wherein the one or more over-expressed genes includes SLC39A4.
72. The method of paragraph 71, wherein the therapeutic agent includes AICAR.
73. The method of any one of paragraphs 33-45, wherein the aneuploid chromosome is 9q, and
wherein the one or more over-expressed genes includes UCK1.
74. The method of paragraph 73, wherein the therapeutic agent includes 6-azacytidine and/or 5-methylcytidine.
75. The method of any one of paragraphs 33-45, wherein the aneuploid chromosome is 10q, and
wherein the one or more over-expressed genes includes ADK.
76. The method of paragraph 75, wherein the therapeutic agent includes Triciribine.
77. The method of any one of paragraphs 33-45, wherein the aneuploid chromosome is 11q.
78. The method of paragraph 77, wherein the wherein the one or more over-expressed genes includes FADS2.
79. The method of paragraph 78, wherein the therapeutic agent includes ML239.
80. The method of paragraph 77, wherein the wherein the one or more over-expressed genes includes SLC35F2.
81. The method of paragraph 80, wherein the therapeutic agent includes YM155.
82. The method of any one of paragraphs 33-45, wherein the aneuploid chromosome is 12p, and
wherein the one or more over-expressed genes includes PDE3A.
83. The method of paragraph 82, wherein the therapeutic agent includes DCEBIO, and/or zardaverine.
84. The method of any one of paragraphs 33-45, wherein the aneuploid chromosome is 16p or 16q.
85. The method of paragraph 84, wherein the one or more over-expressed genes includes ABCC1, optionally wherein the therapeutic agent includes Dasatinib.
86. The method of paragraph 84, wherein the one or more over-expressed genes includes SULT1A1.
87. The method of paragraph 86, wherein the therapeutic agent includes Oncrasin-1.
88. The method of paragraph 84, wherein the one or more over-expressed genes includes NQO1.
89. The method of paragraph 88, wherein the therapeutic agent includes an HSP90 inhibitor.
90. The method of any one of paragraphs 33-45, wherein the aneuploid chromosome is 17p or 17q.
91. The method of paragraph 90, wherein the one or more over-expressed genes includes SLC47A1 or AHR.
92. The method of paragraph 91, wherein the one or more over-expressed genes includes SLC47A1 and the therapeutic agent includes Mitoxantrone, and/or disulfiram; or wherein the one or more over-expressed genes includes AHR and the therapeutic agent includes CGS-15943.
93. The method of paragraph 90, wherein the one or more over-expressed genes includes SLFN11.
94. The method of paragraph 93 wherein the therapeutic agent(s) include DNA-damaging agent(s).
95. The method of paragraph 90, wherein the one or more over-expressed genes includes SLFN12.
96. The method of paragraph 95, wherein the therapeutic agent includes DCEBIO, and/or zardaverine.
97. The method of any one of paragraphs 33-45, wherein the aneuploid chromosome is 19p or 19q.
98. The method of paragraph 97, wherein the one or more over-expressed genes includes DCAF15.
99. The method of paragraph 91, wherein the therapeutic agent includes Indisulam.
100. The method of paragraph 97, wherein the one or more over-expressed genes includes SLC6A16.
101. The method of paragraph 100 wherein the therapeutic agent includes Temsirolimus.
102. The method of any one of paragraphs 33-45, wherein the aneuploid chromosome is 21q, and
wherein the one or more over-expressed genes includes SLC19A1.
103. The method of paragraph 102, wherein the therapeutic agent includes an anti-folate.
104. The method of paragraph 103, wherein the anti-folate includes Methotrexate.
105. A mutant A2058 cell including loss of a trisomic chromosome relative to an A2058 parental cell, optionally loss of 1q trisomy, 7p trisomy, 8q trisomy, or a combination thereof.
106. A mutant A2780 cell including loss of a trisomic chromosome relative to an A2780 parental cell, optionally loss of 1q trisomy.
107. A mutant AGS cell including loss of a trisomic chromosome relative to an AGS parental cell, optionally loss of 1q trisomy.
108. A mutant MCF10A cell including loss of a trisomic chromosome relative to an MCF10A parental cell, optionally loss of 1q trisomy.
109. A mutant HCT116 cell including loss of a trisomic chromosome relative to an HCT116 parental cell, optionally loss of 8q trisomy.
110. A clone or cell thereof selected from the clones of Table 2.
111. An sgRNA encoded by a nucleic acid sequence Table 4A or 4B.
112. A method for creating a viable isogenic diploid-like cell or tissue from an aneuploid cell or tissue,
   including:
(i) introducing into an aneuploid cell or tissue including an aneuploid chromosome arm a gene editing composition configured to cleave the aneuploid chromosome arm; and optionally a nucleic acid configured to integrate into the aneuploid chromosome arm to form isogenic diploid-like cell or tissue disomic for the aneuploid arm; and
(ii) optionally isolating the isogenic diploid-like cell or tissue optionally wherein the aneuploid chromosome arm is lost from the cell or tissue.
113. A method of treating a disease or disorder associated with an aneuploidy in a subject in need thereof, including
(i) screening one or more candidate active agents for selective toxicity against an aneuploid cell or aneuploid tissue obtained from the subject,
wherein the candidate active agents(s) is selected as being selectively toxic to the aneuploid cell or aneuploid tissue when the agent reduces viability or reproduction of the aneuploid cell or aneuploid tissue to a greater extent than a viable isogenic diploid-like cell or tissue prepared according to the method of paragraph 112, and
(ii) administering the selected candidate active agent(s) to the subject in an amount effective to treat the disease or disorder associated with an aneuploidy in the subject.
114. The method of paragraph 113, wherein the selected candidate active agent(s) is activated selectively by the aneuploid cells, and/or is selectively imported into the aneuploid cells, and or has enhanced activity in the aneuploid cells to a greater extent than into isogenic diploid like cells.
115. The method of paragraph 113 or 114, wherein the active agent is non-toxic to isogenic diploid-like cells.
116. The method of paragraph 113 or 114 or 115, wherein the method further includes administering a second or further active agent to the subject to treat the disease or disorder associated with an aneuploidy in the subject.
117. The method of paragraph 116, wherein the selected candidate active agent(s) selectively sensitizes the aneuploid cancer cells to the second or further active agent.
118. The method of paragraph 116 or 117, wherein the selected candidate active agent(s) enhances the cellular import of the second or further active agent into the aneuploid cancer cells.
119. A method for creating a viable isogenic cell or tissue having an ablated chromosome from a precursor cell or tissue, including:
(i) integrating into a precursor cell or tissue including a target chromosome a gene editing composition configured to integrate a reporter gene onto the target chromosome;
   and
(ii) isolating isogenic cells or tissue that lack expression of the reporter gene.
120. The method of paragraph 119, wherein the gene editing composition is selected from the group consisting of Clustered Regularly Interspaced Short Palindromic Repeats/CRISPR associated protein (CRISPR/Cas), zinc finger nucleases (ZFN), and transcription activator-like effector nucleases (TALEN).
121. The method of paragraph 119 or 120, wherein the gene editing composition includes a CRISPR/Cas composition.
122. The method of any one of paragraphs 119-121, wherein the precursor cell or tissue is an aneuploid cell or tissue.
123. The method of any one of paragraphs 119-121, wherein the precursor cell is a disomic cell or tissue, and wherein the isogenic cell or tissue is a monosomic cell or tissue.
124. The method of any one of paragraphs 119-123, wherein the reporter gene is selected from the group consisting of herpesvirus thymidine kinase (HSV-TK), Cytosine Deaminase, Diphtheria Toxin, and a fluorescent protein.
125. The method of paragraph 119-124, wherein the reporter gene is herpesvirus thymidine kinase (HSV-TK), and
wherein isolating in step (ii) includes contacting the cell or tissue with ganciclovir, and isolating an isogenic cell or tissue that is not susceptible to ganciclovir.
126. The method of any one of paragraphs 119-125, wherein the cell or tissue is human, and
wherein the target chromosome is selected from the group consisting of chromosome 1, 2, 3, 4, 5, 6, 7, 8, 9, 10, 11, 12, 13, 14, 15, 16, 17, 18, 19, 20, 21, 22, X, or Y.
127. The method of any one of paragraphs 119-126, wherein the target chromosome expresses one or more genes selected from the group consisting of CYP2J2, MFSD2A, UCK1, UCK2, SLC16A1, UGT1A10, DCK, SLC26A2, SLC29A1, ACTB, ABCB1, SLC39A4, ADK, FADS2, SLC35F2, PDE3A, ABCC1, SULT1A1, NQO1, SLC47A1, SLFN11, SLFN12, DCAF15, SLC6A16, SLC19A1, and AHR.
128. The method of any one of paragraphs 119-127, wherein the precursor cell or tissue is a cancer cell or cancer tissue,
optionally wherein the cancer cell or tissue is an aneuploid cancer cell or tissue.
129. The method of paragraph 128, wherein the aneuploid cancer cell or tissue is derived from a subject with cancer.
130. The method of paragraph 129, further including prior to step (i), obtaining the aneuploid cancer cell or tissue from the subject.
131. The method of any one of paragraphs 119-130, wherein the cancer is selected from the group consisting of anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer, uterine cancer, acute myeloid leukemia, and adrenocortical carcinoma.
132. An isogenic cell or tissue formed from a precursor cell or tissue by the method of any one of paragraphs 119-131.
133. A method for creating a viable isogenic cell or tissue or organoid having an ablated aneuploid chromosome or a fragment thereof from a precursor aneuploid cell or tissue or organoid, including:
(i) introducing into an aneuploid precursor cell or tissue or organoid a gene editing composition configured to cleave an aneuploid chromosome arm and optionally a nucleic acid configured to integrate into the aneuploid chromosome arm to form isogenic cell or tissue or organoid including a cleaved aneuploid chromosome arm;
   and
(ii) optionally isolating the isogenic cell or tissue or organoid,
   optionally wherein the cleaved aneuploid chromosome arm is not present in the isogenic the cell or tissue or organoid.
134. The method of paragraph 133, wherein the gene editing composition includes a CRISPR/Cas composition,
wherein the nucleic acid configured to integrate into the aneuploid chromosome arm includes a reporter gene,
wherein the CRISPR/Cas composition is configured to cleave the aneuploid chromosome arm between the integrated reporter gene and a centromere of the aneuploid chromosome arm; and
wherein isolating the isogenic cell or tissue or organoid includes selecting a cell or tissue or organoid lacking the reporter gene.
135. The method of paragraph 134, wherein the reporter gene is selected from the group consisting of herpesvirus thymidine kinase (HSV-TK), Cytosine Deaminase, Diphtheria Toxin, and a fluorescent protein.
136. The method of paragraph 135, wherein the reporter gene is herpesvirus thymidine kinase (HSV-TK); and
wherein the isolating in step (ii) includes contacting the cell or tissue or organoid with ganciclovir, and isolating an isogenic cell or tissue that is not susceptible to ganciclovir.
137. The method of any one of paragraphs 133-136, wherein the gene editing composition includes a CRISPR/Cas composition,
wherein the nucleic acid configured to integrate into the aneuploid chromosome arm includes a telomere repeat sequence,
wherein the CRISPR/Cas composition is configured to cleave nucleotides proximal to a centromere in the aneuploid chromosome arm and integrate the telomere repeat sequence to provide a de novo telomere; and
wherein isolating the isogenic cell or tissue or organoid includes selecting a cell or tissue or organoid including the de novo telomere.
138. The method of paragraph 137, wherein the telomere repeat sequence includes ˜100 repeats of the human telomere seed sequence, and
optionally a reporter gene.
139. The method of paragraph 137 or 138, wherein the telomere repeat sequence includes a reporter gene, and wherein isolating the isogenic cell or tissue or organoid includes selecting a cell expressing the reporter gene.
140. The method of any one of paragraphs 133 to 139, wherein the cell or tissue is human, and
wherein the aneuploid chromosome arm is selected from the group consisting of arm 1q, 1p, 2q, 2p, 3q, 3p, 4q, 4p, 5q, 5p, 6q, 6p, 7p, 7q, 8p, 8q, 9p, 9q, 10p, 10q, lip, 11q, 12p, 12q, 13p, 13q, 14q, 14p, 15q, 15p, 16p, 16q, 17p, 17q, 18p, 18q, 19q, 19q, 20q, 20p, or 21q, 21p, 22q, 22p, Xp, Xq, Yp, or Yq.
141. The method of any one of paragraphs 133 to 140, wherein the target chromosome arm is selected from the group consisting of chromosome 1q, chromosome 7p, and chromosome 8q.
142. The method of any one of paragraphs 133 to 141, wherein the aneuploid chromosome arm is chromosome 1q.
143. The method of any one of paragraphs 133 to 142, wherein the aneuploid chromosome arm expresses one or more genes selected from the group consisting of CYP2J2, MFSD2A, UCK1, UCK2, SLC16A1, UGT1A10, DCK, SLC26A2, SLC29A1, ACTB, ABCB1, SLC39A4, ADK, FADS2, SLC35F2, PDE3A, ABCC1, SULT1A1, NQO1, SLC47A1, SLFN11, SLFN12, DCAF15, SLC6A16, SLC19A1, and AHR.
144. The method of any one of paragraphs 133 to 143, wherein the precursor cell or tissue is a cancer cell or tissue, optionally wherein the cancer cell or tissue is an aneuploid cancer cell or tissue from an aneuploid cancer.
145. The method of paragraph 144, wherein the aneuploid cancer cell or tissue is derived from a subject with cancer.
146. The method of paragraph 145, further including prior to step (i), obtaining the aneuploid cancer cell or tissue from the subject.
147. The method of any one of paragraphs 144 to 146, wherein the cancer is selected from the group consisting of anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer, uterine cancer, acute myeloid leukemia, and adrenocortical carcinoma.
148. The method of any one of paragraphs 1 to 32, or 119 to 131 or 133 to 147, wherein the precursor cell or tissue is or is part of a tissue graft, a prosthesis or an organoid.
149. An isogenic cell or tissue formed from a precursor cell or tissue by the method of any one of paragraphs 1 to 32, or 133 to 148.
150. A tissue graft, a prosthesis or an organoid comprising the cell or tissue of any one of paragraphs 132 or 149.

### EXAMPLES

Girish, et al., “Oncogene-like addiction to aneuploidy in human cancers,” Science 0,eadg4521DOI:10.1126/science.adg4521, and Version 1. bioRxiv. Preprint. 2023 Jan. 10. doi: 10.1101/2023.01.09.523344, pages 1-94, and including all supplement material are specifically incorporated by reference herein in its entirety.

### Example 1: Development of Chromosome Engineering Tools for Elimination of Aneuploidies from Cancer Genomes

### Strategy

An experimental approach to kill cancer cells carrying extra chromosomes was sought. Typically, these extra chromosomes encode certain genes whose overexpression increases sensitivity to specific anti-cancer compounds. Potential examples of sensitivity-enhancing genes include drug importer pumps and enzymes that convert pro-drugs to active drugs. Results presented below show that cancers that harbor extra copies of chromosomes encoding these genes can be more sensitive to drugs that rely on the affected pathways.

### Methods

### Cell Lines and Culture Conditions

The identities of all cell lines used in this study were confirmed using STR (short tandem repeat) profiling (University of Arizona Genetics Core). A2780 was grown in RPMI 1640 medium (Gibco, cat. no. 11875119) supplemented with 10% fetal bovine serum (FBS) (Sigma-Aldrich, cat. no. F4135), 2 mM glutamine (Lonza, cat. no. 17-605F), and 100 U/mL penicillin-streptomycin (Life Technologies, cat. no. 15140122). A2058, HCT116, HEK-293T, and PLAT-A cells were grown in DMEM (Gibco, cat. no. 11995073) supplemented with 10% FBS, 2 mM glutamine, and 100 U/mL penicillin-streptomycin. AGS was grown in F-12K (ATCC; cat. no. 30-2004) supplemented with 10% FBS, 2 mM glutamine, and 100 U/mL penicillin-streptomycin. HAP1 cells were grown in IMDM (Gibco, cat. no. 12440053) supplemented with 10% FBS, 2 mM glutamine, and 100 U/mL penicillin-streptomycin. MCF-10A was grown in DMEM/F-12 (Gibco, cat. no. 11320082), supplemented with 5% horse serum (Gibco, cat. no. 16050122), 20 ng/mL EGF (PeproTech, cat. no. AF-100-15), 0.5 ng/mL hydrocortisone (Sigma-Aldrich, cat. no. H0888), 100 ng/mL cholera toxin (Sigma-Aldrich, cat. no. C8052), 10 μg/mL insulin (Sigma-Aldrich, cat. no. I1882), 5 mM transferrin (Sigma-Aldrich, cat. no. T8158), and 100 U/mL penicillin-streptomycin. All cells were cultured in a humidified environment at 37° C. and 5% CO2.

### Production of Lentivirus and Retrovirus

HEK293T (lentivirus) or PLAT-A (retrovirus) cells were transfected using the calcium-phosphate method (Giuliano, et al., Curr. Protoc. Mol. Biol. 128, e100 (2019)). Virus-containing supernatant was harvested 48 to 72 hours post-transfection, filtered through a 0.45 m syringe, and then frozen at −80° C. for later use or applied directly to cells with 4-10 g/mL polybrene (Santa Cruz Biotechnology, Inc., cat no. SC-134220). The culture medium on target plates was changed 24 hours post-transduction.

### CRISPR Plasmid Cloning

Guide RNAs for CRISPR experiments were designed with Benchling (available at world wide web address: “benchling.com”). Guides were cloned into the Lenti-Cas9-gRNA-GFP vector (Addgene #124770) or LRCherry2.1 (Addgene #108099) using a BsmBI digestion as previously described (Giuliano, et al., Curr. Protoc. Mol. Biol. 128, e100 (2019)). Plasmids were transformed in Stbl3 E. coli (Thermo Fisher Scientific, cat. no. C737303) and sequence-verified to confirm the presence of the correct gRNA. CRISPR gRNA sequences are listed in Table 4A.

### CRISPRi Plasmid Cloning

Guide RNAs for CRISPRi experiments were chosen from refs (Horrbeck, et al., eLife. 5, e19760 (2016), Sanson, et al., Nat. Commun. 9 (2018)) and cloned into the LRG2.1 mCherqy vector (Addgene #108099) as described above. Plasmids were transformed in Stbl3 E. coli and sequenced to confirm the presence of the correct gRNA sequence. CRISPRi gRNA sequences are listed in Table 4B.

TABLE 4A
CRISPR gRNA sequences

| | | SEQ | |
|---|---|---|---|
| | | ID | Genomic |
| Guide RNA | Sequence | NO: | Target |
| AAVS1_gRNA | ACTGTTGACGGCGGCGATGT | 3 | chr19: 55112543 |
| Chr1q_Cassette-Integration_gRNA | AGAACAGTTGAAGTCCTT | 4 | chr1: 150092442 |
| Chr1q_Cassette-Proximal_gRNA | GTAACAACATGTAGTACCG | 5 | chr1: 150157781 |
| Chr1q_Centromere-Targeting_gRNA | CAGGTGGGTTACAAAAGAC | 6 | chr1: 150157781 |
| Chr7_Centromere-Targeting_gRNA | AGTAAACTCCAACAGACCTG | 7 | chr7: 51599239, |
| | TGATTCAATTGGAAGCCAAT | 8 | chr7: 61041866 |
| Chr8q_Centromere-Targeting_gRNA | CAGGTGGGTTACAAAAGAC | 9 | chr8: 47217869 |
| MDM4_Deletion_Downstream_gRNA_1 | CAGCTTCTGAGGCTCTACCG | 10 | chr1: 204566478 |
| MDM4_Deletion_Downstream_gRNA_2 | CTAGGTTACAGATAGTCTCG | 11 | chr1: 204566284 |
| MDM4_Deletion_Upstream_gRNA_1 | TCAATGGTAAAATAGATCGA | 12 | chr1: 204507400 |
| MDM4_Deletion_Upstream_gRNA_2 | AGCTATCGGGTTGCACTGTG | 13 | chr1: 204507505 |
| OR2M4_Deletion_gRNA | CAAATGACCCACTTGTCCGA | 14 | chr1: 248231419, |
| | TGTTCAATGGAGATGTCCCG | 15 | chr1: 248248568 |
| OR6K6_Deletion_gRNA_1 | GTAGGTATAGCAGACCACTG | 16 | chr1: 158749288, |
| | TGCCCCACATTGTACCACAG | 17 | chr1: 158774492 |
| OR6K6_Deletion_gRNA_2 | GATTTTGGTAGTCATGACGA | 18 | chr1: 158744885, |
| | ATTATACCCAGCCATTCCCG | 19 | chr1: 158772138 |
| OR6K6_Deletion_gRNA_3 | GCGGAATCCTAATGACCAGG | 20 | chr1: 158740163, |
| | ATGATGCCACAATCACGTGA | 21 | chr1: 158756793 |
| Rosa26_gRNA | ACAGCAAGTTGTCTAACCCG | 22 | chr3: 395266 |
| TP53-KO_gRNA_1 | AGATGGCCATGGCGCGGAC | 23 | chr17: 7675125 |
| TP53-KO_gRNA_2 | GCATGGGCGGCATGAACCG | 24 | chr17: 7674220 |

TABLE 4B
CRISPRi gRNA sequences

| | | SEQ ID | Target |
|---|---|---|---|
| Guide RNA | Sequence | NO: | Gene |
| AAVS1 gRNA_1 | ACTGTTGACGGCGGCGATGT | 25 | AAVS1 |
| AAVS1 gRNA_2 | GCTGATACCGTCGGCGTTGG | 26 | AAVS1 |
| MDM4 CRISPRi gRNA_1 | CTCCCCCGCCGAGCCCGGGT | 27 | MDM4 |
| MDM4 CRISPRi gRNA_2 | CAAAATGCTGCCGAGGCCCT | 28 | MDM4 |
| PIP5K1A CRISPRi gRNA_1 | GGTTAGGAAGGACGGAGAAG | 29 | PIP5K1A |
| PIP5K1A CRISPRi gRNA_2 | TTAACAGGCCGTGGTTAGGA | 30 | PIP5K1A |

### Cloning of Doxycycline-Inducible Plasmids

The MDM4 coding sequence (NM_002393.5) was cloned into Lenti-X™ Tet-One™ Inducible Expression System (Takara Bio, cat. no. 631847). The resulting plasmid was then subject to whole-plasmid sequencing for verification. A2780 cells were transduced with pLVX-TetOne-MDM4-Puro and selected for with 1.5 g/mL puromycin (InvivoGen, cat. no. ant-pr-1). Tight inducibility was confirmed through doxycycline (Sigma-Aldrich, cat. no. D3072) titration and quantitative real-time PCR. The final plasmid was deposited on Addgene (#195140). Similarly, coding sequences for BCL9 (NM_004326.4), MCL1 (NM_021960.5), PFDN2 (NM_012394.4), PPP1R15B (NM_032833.5), and PSMD4 (NM_002810.4) were cloned into the pLXV-TetOne backbone and subject to sequence verification. Transduction and selection were performed as described above, and the final plasmids were deposited on Addgene (#202662-202666).

Libraries for genomic copy number analysis were prepared as described in ref (Wang, et al., Genome Res. 26, 844-851 (2016)). In brief, genomic DNA was enzymatically fragmented to a mean size of ˜40 bp and ligated to generate long chimeric DNA molecules (˜300-700 bp) for sequencing. Fragment size selection and purification were done with Agencourt AMPure XP beads (Beckman Coulter, cat. no. A63881). Illumina-compatible NEBNext Multiplex Dual Index primer pairs and adapters (New England Biolabs, cat. no. E6440S) were added to each sample, and the products were pooled for next-generation sequencing (NGS). Libraries were sequenced using an Illumina MiSeq or NovoSeq sequencer. The generated reads were demultiplexed then mapped using a custom Nextflow (Di Tommaso, et al., Nat. Biotechnol. 35, 316-319 (2017)) wrapper running SMASH built from MUMdex (Andrews, ET AL., bioRxiv, 078261 (2016)) (commit: 25e1f2f). Plots were generated via a custom script utilizing tidyverse (Wickham, ET AL., J. Open Source Softw. 4, 1686 (2019)) (v1.3.1) components run in the R programming environment (R Core Team 2022; v4.2.0).

### G-Banding Karyotyping

A2780 samples were sent to Cell Line Genetics Inc. (available at world wide web address: “clgenetics.com”), and AGS and A2058 samples were sent to Karyologic Inc. (available at world wide web address: “karyologic.com”) for G-banding karyotyping. A minimum of 10 metaphase spreads per sample were counted and analyzed to prepare representative karyotype spreads.

### TaqMan Copy Number Analysis

Genomic DNA was extracted and isolated using the Qiagen DNeasy Blood & Tissue kit (Qiagen, cat. No. 69506). Reactions were prepared in quadruplicate, and target probes were duplexed with RNaseP (Applied Biosystems, cat. no. 4316831) or TERT (Applied Biosystems, cat. no. 4403316) as the reference assay. Quantitative PCR was performed using TaqPath ProAmp Master Mix (Applied Biosystems, cat. no. A30867) and quantified using the QuantStudio 6 Flex Real-Time PCR system (Applied Biosystems). Copy number analysis was performed as described in ref (Mayo, et al., Curr. Protoc. Hum. Genet., in press, doi:10.1002/0471142905.hg0213s67) and copy number calls were normalized to the near-diploid colorectal cancer cell line DLD1. To screen for chromosome arm loss, a minimum of three probes spanning the chromosome of interest were used, and the copy number calls for the individual probes were averaged to determine chromosome arm copy number. To screen for single copy gene deletions, two probes for the gene of interest were used, and the copy number calls for the individual probes were averaged to determine gene copy number.

### Gene Expression Analysis: RNA-Seq

Total cellular RNA was extracted using the Qiagen RNeasy Mini Kit (Qiagen, cat. no. 74106) and subjected to on-column DNase digestion (Qiagen, cat. no. 79254). Purified RNA samples were submitted to Novogene for RNA-seq and quantification. In brief, mRNA was purified from total cellular RNA using poly-T oligo-attached magnetic beads. After fragmentation, first strand cDNA was synthesized using random hexamer primers, followed by second strand cDNA synthesis. Subsequently, libraries were prepared with end repair, A-tailing, adapter ligation, size selection, amplification, and purification. Libraries were sequenced on an Illumina platform and paired-end reads were generated.

Raw data (raw reads) in fastq format were firstly processed through in-house Novogene Perl scripts to remove adapters, reads containing >10% undetermined bases, and low-quality reads (>50% of bases with Qscore≤5). Reference genome and gene model annotation files were downloaded from genome website directly. An index of the reference genome was built using Hisat2 v2.0.5 and paired-end clean reads were aligned to the hg38 reference genome using Hisat2 v2.0.5. featureCounts v1.5.0-p3 was used to count the reads numbers mapped to each gene. The FPKM of each gene was calculated based on the length of the gene and reads count mapped to this gene. Read counts were normalized across samples via TMM normalization using edgeR v3.40.2 and grouping by ploidy (Robinson, et al., Bioinforma. Oxf Engl. 26, 139-140 (2010)). Gene Set Enrichment analysis was performed with the local version of the GSEA analysis tool available at world wide web address: “broadinstitute.org/gsea/index.jsp”, all Hallmark gene sets, and genes were ranked according to Signal2Noise. Following analysis by the GSEA analysis tool, select plots were reproduced using enrichplot v1.18.3 and clusterProfiler v4.6.2 in addition to tabulating enrichment values for all Hallmark gene sets (Yu, et al., Visualization of Functional Enrichment Result (2023), Wu, et al., Innov. Camb. Mass. 2, 100141 (2021)).

### Mass Spectrometry

Proteomic analysis was conducted as previously described (Liu, et al., Nat. Biotechnol. 37, 314-322 (2019)). In short, cell pellets were thawed and a VialTweeter device (Hielscher-Ultrasound Technology) was used to sonicate the samples (4° C.; 1 min; two cycles). The samples were centrifuged at 20,000 g for 1 hour to remove insoluble material. Protein concentration was measured using the Bio-Rad protein assay dye (Bio-Rad, cat. no. 5000006). 800 μg of protein per sample were diluted (final concentration=2 μg/μL) using a 10 M urea/100 mM ammonium bicarbonate buffer, reduced by 10 mM DTT (1 hour; 56° C.), and alkylated by 20 mM IAA (1 hour; RT). The proteins were subjected to a precipitation-based digestion (Collins, et al., Nat. Commun. 8, 291 (2017)). Briefly, five volumes of precooled precipitation solution (50% acetone, 50% ethanol, and 0.1% acetic acid) were added to the samples. After overnight incubation at −20° C., the samples were centrifuged (20,000×g; 4° C.; 40 min). The precipitate was washed with precooled 100% acetone, centrifuged (20,000×g; 4° C.; 40 min), and the remaining acetone was evaporated in a SpeedVac. For protein digestion, 300 μL of 100 mM NH4HCO3 with sequencing grade porcine trypsin (Promega) at a trypsin-to-protein ratio of 1:20 were added and incubated overnight at 37° C. The resulting peptide samples were acidified with formic acid and desalted using a C18 column (MarocoSpin Columns, NEST Group INC.) according to the manufacturer's instructions. 1 μg of the peptide mixture was used for the LC-MS analysis as described previously (Liu, et al., Nat. Biotechnol. 37, 314-322 (2019), Mehnert, et al., Proteomics. 19, e1800438 (2019)). The LC separation was performed using an EASY-nLC 1200 system (Thermo Scientific) using a self-packed PicoFrit column (New Objective, Woburn, MA, USA; 75 μm×50 cm length) with a ReproSil-Pur 120A C18-Q 1.9 μm resin (Dr. Maisch GmbH, Ammerbuch, Germany). A 120-min gradient length was used to elute peptides from the LC; with buffer B (80% acetonitrile containing 0.1% formic acid) from 5% to 37% and corresponding buffer A (0.1% formic acid in H2O). The flow rate was 300 nL/min, and the temperature was controlled at 60° C. using a column oven (PRSO-V1, Sonation GmbH, Biberach, Germany). The Orbitrap Fusion Lumos Tribrid mass spectrometer (Thermo Scientific) coupled with a NanoFlex ion source (spray voltage of 2000 V, 275° C.) was used for the MS analysis. The method for DIA-MS consisted of a MS1 survey scan and 33 MS2 scans of variable windows (Bruderer, et al., Mol. Cell. Proteomics MCP. 16, 2296-2309 (2017), Bruderer, et al., Mol. Cell. Proteomics MCP. 18, 1242-1254 (2019)). The MS1 scan parameters were set as follows: scan range 350-1650 m/z, resolution 120,000 at m/z 200, the AGC target 2.0E6, and the maximum injection time 100 ms. The normalized HCD collision energy was 28%. The MS2 scan parameters were the following: resolution 30,000 at m/z 200, the AGC target 1.5E6, and the maximum injection time 50 ms. The default peptide charge state was set to 2. Both of MS1 and MS2 spectra were recorded in a profile mode.

DIA-MS data analysis was performed using Spectronaut v15 (Bruderer, et al., Mol. Cell. Proteomics MCP. 16, 2296-2309 (2017), Bruderer, et al., Mol. Cell. Proteomics MCP. 14, 1400-1410 (2015)) using the library-free DirectDIA workflow (Bruderer, et al., Mol. Cell. Proteomics MCP. 16, 2296-2309 (2017), Tsou, et al., Nat. Methods. 12, 258-264, 7 p following 264 (2015)) and the Swiss-Prot protein database (September 2020, 20,375 entries). The analysis was performed using default Spectronaut settings. Methionine oxidation and N-terminal acetylation were set as variable modifications, where carbamidomethylation at cysteine was set as a fixed modification. Both peptide- and protein-FDR were controlled at 1%, and the resulting data matrix was filtered by “Qvalue”. The DIA quantification was performed using the MS2 level peak areas. Protein intensities were exported, log 2-transformed and normalized using LOESS normalization prior to the subsequent analysis.

### Quantitative Real-Time PCR

Total cellular RNA was extracted and isolated using the Qiagen RNeasy Mini Kit (Qiagen, cat. no. 74106). cDNA synthesis was performed using SuperScript IV VILO Master Mix (Invitrogen, cat. no. 11756500). Quantitative PCR was performed for the target genes using TaqMan Fast Advanced Master Mix (Applied Biosystems, cat. no. 4444963) and quantified using the QuantStudio 6 Flex Real-Time PCR system (Applied Biosystems). qPCR primers are listed in Table 5.

TABLE 5
Primers

| Primer Name | Primer Sequence | SEQ ID NO: |
|---|---|---|
| Chr1q Forward Ultramer | TTTCATAGACGTTCTTCCTTAAGTCAGCTTG | 31 |
| | AATAAGTAGTAGGAGGAGGCTCAGGAAAACA | |
| | TTTATTAATGTTTTCATAACCAAATGTGACA | |
| | TAGTCATCTTTGCGAGGTACACAATTGAGCC | |
| | AAGCTCTAAGAGCCTATCCCTGTATCTAGGT | |
| | TTTGTCCTTACATGTATATAACCTAgacatt | |
| | gattattgactagt | |
| Chr1q Reverse Ultramer | TCACCATAGCTATCCTCTGACCATCACCTGC | 32 |
| | CTGGACAATATATCTGTCTCTTATGGAAAAG | |
| | AAAGGCAAGAACTGGGAAGGAACAAATTCAA | |
| | GTGGGCTTTTAGCATAGTAAGAGAACACTGT | |
| | CCTTCAGTCTGATCATTTCAGGGTGGCAGGG | |
| | TATTTTTGTACTACTGGAGAGGAAAccatag | |
| | agcccaccgcatcc | |
| MDM4 qPCR_1F | TGATTGTCGAAGAACCATTTCGG | 33 |
| MDM4 qPCR_1R | TGCAGGGATCAAAAAGTTTGGAG | 34 |
| MDM4 qPCR_2F | TTTTGATCCCTGCAACTCAGTG | 35 |
| MDM4 qPCR_2R | GTCTCGTGGTCTTTTCTCACAT | 36 |
| GAPDH qPCR_F | TGCACCACCAACTGCTTAGC | 37 |
| GAPDH qPCR_R | GGCATGGACTGTGGTCATGAG | 38 |
| Cassette Integration 5′ | GAGCTAGCTGCTGTTTTCTAATGTTTGC | 39 |
| Check_F | | |
| Cassette Integration 5′ | cataaaaggaaactttcggagcgc | 40 |
| Check_R | | |
| Cassette Integration 3′ | TCAGTTAGCCTCCCCCATCTC | 41 |
| Check_F | | |
| Cassette Integration 3′ | GTAATGACAAGCGCCCAGATAAC | 42 |
| Check_R | | |
| Cassette Deletion Check_F | ggactctcagcaattaaactg | 43 |
| Cassette Deletion Check_R | cctgtattctccactctacc | 44 |
| KRAS Exon 2_1F | cgtcgatggaggagtttgtaaatg | 45 |
| KRAS Exon 2_1R | gcacagagagtgaacatcatgg | 46 |
| KRAS Exon 2_2F | cgatacacgtctgcagtcaactg | 47 |
| KRAS Exon 2_2R | gacatactcccaaggaaagtaaagttccc | 48 |

### Western Blotting

One day prior to lysate harvest, 500,000 cells were seeded in a six-well plate. Whole cell lysates were harvested and resuspended in RIPA buffer [25 mM Tris, pH 7.4, 150 mM NaCl, 1% Triton X 100, 0.5% sodium deoxycholate, 0.1% sodium dodecyl sulfate, protease inhibitor cocktail (Sigma, cat. no. 4693159001), and phosphatase inhibitor cocktail (Sigma, cat. no. 4906845001)]. Protein concentration was quantified using the RC DC Protein Assay (Bio-Rad; cat. no. 500-0119). Equal amounts of lysate were denatured and loaded onto a 10% SDS-PAGE gel. The Trans-Blot Turbo Transfer System (Bio-Rad) and polyvinylidene difluoride membranes were used for protein transfer. Antibody blocking was done with 5% milk in TBST (19 mM Tris base, NaCl 137 mM, KCl 2.7 mM and 0.1% Tween-20) for 1 hour at room temperature. The following antibodies and dilutions were used: p21 (Abeam; cat. no. ab109520) at a dilution of 1:1000 in 5% milk, p53 (Abeam; cat. no. ab1101) at a dilution of 1:1000 in 5% milk, p53 (Abeam; cat. no. ab32389) at a dilution of 1:1000 in 5% milk, phospho-p53 (Ser15) (Cell Signaling, cat. no. 9284S) at a dilution of 1:1000 in 5% milk, cleaved PARP (Asp214) (Cell Signaling; cat. no. 5625S) at a dilution of 1:1000 in 5% milk, and UCK2 (proteinTech; cat. no. 10511-1-AP) at a dilution of 1:1000 in 5% milk. Blots were incubated with the primary antibody overnight at 4° C. Anti-GAPDH (Santa-Cruz; cat. no. sc-365062) at a dilution of 1:20,000 in 5% milk, or anti-alpha tubulin (Sigma-Aldrich; cat. no. T6199) at a dilution of 1:20,000 in 5% milk was used as a loading control. Membranes were washed at room temperature three times (20 mins each) before they were incubated in secondary antibodies for an hour at room temperature. HRP goat anti-mouse (Bio-Rad; cat. no. 1706516) at 1:20,000 was used for tubulin, p53 (ab1101), MDM4, and GAPDH blots while HRP goat anti-rabbit (Abeam; cat. no. ab6721) at 1:20,000 was used for all other primary antibodies. Membranes were washed three times again (20 min each) and developed using ProtoGlow ECl (National Diagnostics; cat. no. CL-300) and autoradiographic film (Lab Scientific; XAR ALF 2025).

### Chromosome Engineering: Chromosome Elimination: Redact-Ns

### Generation of Selection Cassette

A centromere-proximal CRISPR gRNA was designed and cloned into the Lenti-Cas9-gRNA-GFP vector (Addgene #124770) for integration of the selection cassette. Homology arms for cassette integration were designated as the 180 bp immediately upstream and downstream of the guide RNA targeting site. Adapters for PCR were added to the 3′ end of the 180 bp homology arms (Forward: GACATTGATTATTGACTAGT (SEQ ID NO:1); Reverse: CCATAGAGCCCACCGCATCC (SEQ ID NO:2)), and the resulting 200 bp ultramers were obtained from IDT. PCR for selection cassette production was performed with SeqAmp DNA Polymerase (Takara Bio, cat. no. 63850) using the ultramers as primers and AAT-PB-CD2APtk (Addgene #86004) or AAT-PB-PG2APtk (84) (Addgene #195124) as the template. The PCR products were purified and concentrated with the QIAquick PCR Purification kit (Qiagen, cat. no. 28106). Homology arm and ultramer sequences are listed in Table 5.

Knocking-In Cassettes with CRISPR-Mediated HDR

Cells were transfected with the PCR-purified selection cassette and integration gRNA CRISPR plasmid using Lipofectamine 3000 (Invitrogen, cat. no. L3000015) for A2780, AGS & HCT116 cells or FuGENE HD (Promega, cat. no. E2311) for A2058 cells. Integration of the cassette was selected with puromycin (InvivoGen, cat. no. ant-pr-1). Following selection, dsRed positive cells (when using Addgene #86004) or GFP positive cells (when using Addgene #195124) were single cell sorted onto 96-well plates, and clonal cell lines were established. Successful cassette integration was confirmed through PCR and sequencing. Integrant clones were subject to karyotypic validation through SMASH-Seq prior to inducing chromosome arm loss.

### Inducing Chromosome Arm Loss

A centromere targeting CRISPR gRNA was designed and cloned into the Lenti-Cas9-gRNA-GFP vector (Addgene #124770) for targeted chromosome elimination. Integrant clones were transfected with the centromere targeting gRNA CRISPR plasmid using Lipofectamine 3000 or FuGENE HD. Chromosome arm loss was selected for with 10 μM ganciclovir (Sigma Aldrich, cat. no. G2536). Following ganciclovir selection, dsRed or GFP negative cells were single cell sorted onto 96-well plates, and clonal cell lines were established. Clones were screened for targeted chromosome arm loss with TaqMan copy number assays as described below and chromosome elimination was confirmed through SMASH karyotyping. The aneuploidy-loss cell lines generated using this method are listed in Table 2.

### Chromosome Elimination: ReDACT-TR

### Generation of the Artificial Telomere Construct

The ReDACT-TR technique was motivated by the use of a telomere seed sequence to generate monosomic cells as described in ref (Taylor, et al., Cancer Cell. 33, 676-689.e3 (2018)). To enhance the efficacy of this approach, plasmids linking a telomere seed sequence with a puromycin selection marker were created, which allowed us to enrich for stably-transfected aneuploidy-loss cells using drug selection. These EF1a-Puro-Telo vectors (Addgene #195138 and #195139) were generated by introducing a puromycin selection marker to a telomere seed sequence previously described in ref (Taylor, et al., Cancer Cell. 33, 676-689.e3 (2018)). This vector was digested with FastDigest KpnI (Thermo Fisher Scientific, cat. no. FD0524) and FastDigest BstZ17I (Thermo Fisher Scientific, cat. no. FD0704), and gel purified to obtain the artificial telomere construct.

Inducing Chromosome Arm-Loss with CRISPR-Mediated NHEJ

Cells were transfected with the purified artificial telomere construct and centromere-targeting gRNA CRISPR plasmids using Lipofectamine 3000 for AGS cells, or Fugene HD for MCF10A cells. Telomere replacement was selected for with puromycin, and puromycin resistant cells were single cell sorted onto 96-well plates. Clonal cell lines were established and screened for targeted chromosome arm loss with TaqMan copy number assays. Chromosome elimination was confirmed through SMASH karyotyping. The aneuploidy-loss cell lines generated using this method are listed in Table 2.

### Chromosome Elimination: ReDACT-CO

Cells were transfected with centromere targeting gRNA CRISPR plasmids using Lipofectamine 3000 or FuGENE HD. GFP+ cells were single cell sorted onto 96-well plates. Clonal cell lines were established and screened for targeted chromosome arm loss with TaqMan copy number assays. Chromosome elimination was confirmed through SMASH karyotyping. Chr1q_Centromere_Targeting_gRNA (Addgene #195125) and Chr1q_Cassette-Integration_gRNA (Addgene #195126) were used together to target chromosome 1q, Chr7_Centromere-Targeting_gRNA (Addgene #195129) was used to target chromosome 7p, and Chr8q_Centromere-Targeting_gRNA (Addgene #195128) was used to target chromosome 8q (Table 4A). The aneuploidy-loss cell lines generated using this method are listed in Table 2.

### Choice of ReDACT Techniques for Each Cell Line

No underlying reasons motivated the selection of a specific ReDACT technique for each cell line. The first approach, ReDACT-NS, was driven by the historical use of thymidine kinase for genomic modification in embryonic stem cells (Mansour, et al., Nature. 336, 348-352 (1988), Li, et al., Cell Stem Cell. 11, 615-619 (2012)). Several reports were published demonstrating accidental CRISPR-mediated chromosome loss without drug selection (Zuccaro, et al., Cell. 183, 1650-1664.e15 (2020), Papathanasiou, et al., Nat. Commun. 12, 5855 (2021)). ReDACT-TR and ReDACT-CO techniques were also attempted. Consequently, for most cell lines described, multiple ReDACT approaches were initiated at the same time, and then focused on characterizing the clones in which it was possible to verify aneuploidy-loss first. Over time, it was discovered that each technique had certain benefits and drawbacks. For instance, with ReDACT-NS, once a clone was isolated containing on-target integration of the HSV-TK cassette, it was relatively straightforward to produce a large number of aneuploidy-loss clones. In contrast, ReDACT-CO required only one round of single-cell cloning and was much faster than ReDACT-NS, but the overall efficiency of ReDACT-CO tended to be lower than ReDACT-NS. Importantly, consistent results on the effects of aneuploidy-loss regardless were obtained of the ReDACT technique that was ultimately used, underscoring the conclusion that aneuploidy-loss itself is the root cause of the observed phenotypes.

### Generation of DLD1 Trisomy 1 Cells

The DLD1 trisomy 1 clone was generated as previously described in ref (Vasudevan, et al., Dev. Cell. 52, 413-428.e6 (2020)). In brief, DLD1 cells were transiently treated with 1 μM AZ3146 (Selleck Chemicals; cat. no. S2731) for 24 hours, and then the drug was washed out and the cells were allowed to recover for 24 hours. Following the recovery passage, single-cell sorting was performed to isolate individual aneuploid clones. SMASH-Seq was used to identify a clone harboring a trisomy of chromosome 1.

### Phenotypic Characterization of Aneuploidy-Loss Cells: Proliferation Assays

Cells were seeded in 6-well plates at 100,000 cells per well. After 72 hours, cells were harvested, counted, and 100,000 cells were re-plated in fresh medium on 6-well plates. Cells were passaged ten times, and population doublings were calculated at each passage.

### Soft Agar Assays

To assay colony formation in soft agar, a solution of 1.0% Difco Agar Noble (VWR Scientific, USA, cat. no. 90000-772) in sterile water was prepared. The 1% agar solution was mixed 1:1 with the base cell culture medium supplemented with 20% FBS, 4 mM glutamine, and 200 U/mL penicillin/streptomycin, or for MCF10A, supplemented with 10% horse serum, 40 ng/mL EGF, 1 ng/mL hydrocortisone, 200 ng/mL cholera toxin, 20 μg/mL insulin, 10 mM transferrin, and 200 U/mL penicillin streptomycin. 1 mL of this mixture was plated on each well of a 6-well plate and allowed to solidify at room temperature to form a base layer of 0.5% agar. Cells were then harvested and counted. For A2780 and A2058, 20,000 cells were seeded in 0.35% agar (1:1 mixture of 0.7% agar in sterile water and 2x supplemented growth medium). For AGS and MCF10A, 10,000 cells were seeded in 0.35% agar. For HCT116, 35,000 cells were seeded in 0.30% agar. Plates were left at room temperature to solidify and then placed in a humidified incubator at 37° C. and 5% CO2. 1 mL of normal growth medium was added the next day, and every three days after. For soft agar assays involving doxycycline-inducible expression, cells were seeded in agar as described above, with the addition of 1 μg/mL doxycycline (Sigma-Aldrich, cat. no. D3072) to the base cell culture medium and to the normal growth medium replenished every three days. After 10-14 days, cells were fixed with 100% methanol, and stained with 0.01% crystal violet dissolved in 25% methanol. Colony formation was quantified by capturing z-stacks of several fields of view per well from at least three wells on a LSM 710 confocal microscope (Zeiss) or a Cytation5 imaging reader (BioTek Instrument, inc.), under either 4x, 5×, or 6× magnification. The average number of colonies was calculated by counting total number of colonies per field of view from multiple fields of view from at least three wells.

### Xenograft Assays

To assay tumor formation, cells were harvested and resuspended in cold PBS. For A2780 xenograft experiments, 3 million cells were injected in each flank of NU/J mice (Jackson Laboratory, cat. no. 002019). For A2058 xenograft experiments, 2 million cells were injected in each flank in NU/J mice. For HCT116 xenograft experiments, 4 million cells were injected in J:NU mice (Jackson Laboratory, cat. no. 007850). For MCF10A cells expressing HRASG12V, 10 million cells were injected in each flank in NU/J mice. For AGS xenograft experiments, the following conditions were tried: 5 million cells resuspended in PBS in each flank of NU/J mice (Jackson Laboratory, cat. no. 002019); 4 million cells resuspended in PBS in each flank of NGS mice (Jackson Laboratory, cat. no. 005557); and 15 million cells resuspended in a 1:1 PBS:Matrigel mixture in each flank of J:NU mice (Jackson Laboratory, cat. no. 007850). Cells were subcutaneously injected using a 1 mL 25G×⅝ syringe (BD, cat. no. 309626). Mice were visually monitored for tumor formation routinely following injection. Once a tumor was visible, it was measured every three days by calipers. Tumor volume was calculated using the formula V=½ (longer axis)(shorter axis)\hat{ }2. The mice were euthanized if their total tumor burden reached 2000 mm3. The sample sizes for each mouse experiment are included in the “Sample Sizes and Statistical Techniques” section. In certain circumstances, mice were excluded from analysis for ethical reasons: some mice were euthanized before the end of the experiment and excluded from the tumor size calculations because their tumors ulcerated or grew too rapidly and compromised their wellbeing. In almost all cases in which a mouse was removed from the experiment and euthanized prematurely, it was due to the growth of the aneuploid cell line and not the aneuploidy-loss cell line. All mouse protocols were approved by the CSHL and Yale Institutional Animal Care and Use Committees.

### Derivation and Characterization of Cell Lines Post-Xenograft

To derive post-xenograft cell lines, the tumors were cut out using a sterilized pair of scissors. The tumors were placed into a chilled conical tube containing 2-3 mL of TrypLE Express Enzyme (Gibco; cat. no. 1260413) and transported on ice to a biosafety hood. The tumors were minced using sterilized scalpels and allowed to dissociate for 20-30 minutes in a 37° C. water bath. Cell culture medium was prepared using 2× the regular concentration of penicillin-streptomycin, and TrypLE was neutralized using an equivalent volume of this cell culture medium. The mixture was filtered through a cell strainer tube (Corning; cat. no. 352235) and plated onto a tissue culture treated 10 cm dish, with cell culture medium added to a total of 10 mL. The following day, upon cell adherence to the dish, the medium was replaced. When cells reached 70-80% confluence, they were frozen down in medium containing 2× the regular concentration of FBS and 10% DMSO. Cell pellets were simultaneously taken for downstream analyses, including TaqMan copy number assays and SMASH karyotyping. For subsequent phenotypic characterization, a fresh vial of cells was thawed and used in soft agar assays as described above.

KRAS genotyping

The sequence of KRAS exon 2 was obtained from GRCh38 using Benchling. Primers were designed to amplify the genomic region surrounding exon 2 (Table 5). PCR was performed with SeqAmp DNA Polymerase (Takara Bio; cat. no. 63850), and PCR products purified and concentrated with the QIAquick PCR Purification kit (Qiagen; cat. no. 28106). PCR products were sent for Sanger sequencing with both forward and reverse primers to Azenta Biosciences. The ratio of wild type to mutant base (G to A) to assess allelic ratio of KRASWT to KRASG13D was assessed using EditR (Kluesner, et al., CRISPR J. 1, 239-250 (2018)).

### Chromosome 1q SNP Genotyping

Heterozygous SNPs on chromosome 1q were obtained from DepMap for A2058 and AGS, and the sequences around these loci were obtained from GRCh38 using Benchling. Primers were designed to amplify the genomic region surrounding these SNPs in OLFML2B and PPFIA4 in A2058, and ASH1L and LGR6 in AGS respectively (Table 5). PCR was performed with SeqAmp DNA Polymerase, and the PCR products were purified and concentrated with the QIAquick PCR Purification kit. PCR products were sent for Sanger sequencing with both forward and reverse primers to Azenta Biosciences. It was validated that these SNPs were heterozygous in the parental 1q trisomy lines and it was discovered that these SNPs had underwent LOH in the 1q disomy lines. In samples with chromosome 1q regain, sequencing was performed at these loci to differentiate between endogenous and exogenous regain. The regain event was determined to be endogenous if the loss of heterozygosity was maintained, whereas exogenous regain events were marked by the re-emergence of heterozygosity. Two representative genes, OLFML2B in A2058, and ASH1L in AGS.

### Cell Cycle Analysis

One day prior to cell cycle analysis, 1.5-2 million cells were plated onto a 10 cm dish. Cells were harvested by trypsinization with TrypLE, followed by centrifugation at 1,000 rpm for 5 minutes, aspiration of the media, and resuspension of the cell pellet in 1 mL PBS. Cells were added dropwise to 4 mL of ice cold 100% ethanol and fixed at −20° C. for 5-15 minutes. Fixed cells were pelleted by centrifugation at 1,000 rpm for 5 minutes, resuspended in PBS containing 0.05% Triton X-100, 10 g/mL RNAse A (Invitrogen, cat no. A32078), and 20 g/mL propidium iodide (Life Technologies, cat. no. P3566), filtered through a cell strainer FACS tube cap. Cells were incubated for 30 minutes at room temperature in the dark. Cell cycle analysis was measured by flow cytometry (Miltenyi Biotech).

### Senescence Assay

Senescence-associated β-galactosidase staining was performed using a senescence β-galactosidase staining kit (Cell Signaling Technology, cat. no. 9860) according to the manufacturer's instructions. Briefly, in a 6-well plate, the medium was discarded, and the cells were rinsed once in PBS. The cells were then fixed in a fixative reagent for 15 minutes at room temperature. The cells were then rinsed twice in PBS and incubated overnight at 37° C. without CO2 in the β-galactosidase staining solution. The cells were imaged and analyzed under a light microscope (20× magnification, Olympus CKX53).

### Nomination of Candidate Genes on Chromosome 1q

A set of 16 genes encoded on chromosome 1q that could potentially contribute to the “1q aneuploidy addiction” phenotype was selected based on three primary criteria. First, it was reasoned that dosage-sensitive drivers of chromosome 1q gains may sometimes also drive selection for focal or sub-chromosomal amplifications. Selected genes were therefore located in or near segments of chromosome 1q that are recurrently subject to focal or sub-chromosomal amplification, as reported in ref. (Beroukhim, et al., Nature. 463, 899-905 (2010)). Second, it was reasoned that candidate driver genes should cause a loss in cell fitness when the expression of that gene is ablated, and so only genes that had negative gene effect scores in CRISPR screens reported on DepMap were considered (Meyers, et al., Nat. Genet. 49, 1779-1784 (2017)). Third, literature searches were conducted on candidate genes, and cellular testing genes that had previously been associated with tumorigenesis were prioritized for. These analyses led us to produce a list of 16 candidate genes encoded on chromosome 1q (Table 4A). In parallel, an analysis of gene expression and mutual exclusivity associated with chromosome 1q indicated that the p53 pathway was affected by 1q-loss, causing us to first focus on MDM4 as a candidate 1q driver.

### CRISPRi Competition Assays

CRISPRi competition assays for candidate 1q driver genes were performed as described in ref (Girish & Sheltzer, Bio-Protoc. 10, e3682-e3682 (2020)). In brief, A2780 cells were transduced with a dCas9-KRAB construct (Addgene #85969). dCas9-KRAB expressing TagBFP+ cells were purified using flow cytometry. For competition assays, dCas9-KRAB expressing cells were transduced with guides in vectors co-expressing mCherry that targeted the candidate genes. Three days post-transduction, cells were subject to flow cytometry analysis (Miltenyi Biotech) to assess the starting percentage of mCherry+ cells. Cells were re-plated, and timepoints were taken every three days through the conclusion of the assay at the fifth timepoint. To normalize for differences in starting percentages of mCherry+ cells, fold change, defined as (starting % positive)/(% positive at timepoint), was calculated at each timepoint. CRISPRi gRNA sequences are listed in Table 4B.

### Assessing Cellular Sensitivity to UCK2 Poisons and Other Anti-Cancer Agents

A 72-hour drug assay was performed to assess cellular sensitivity to different anti-cancer compounds. One day prior to the addition of the drug, 25,000 cells were plated into individual wells of a 6-well plate. The following day, upon cell adherence to the plate, the medium was discarded and replaced with drug-containing medium at varying concentrations. The cells were grown in the presence of the drug for 72 hours. Following this, the cells were harvested and counted. The following drugs were tested: RX-3117 (MedChemExpress; cat. no. HY-15228), 3-deazauridine (Cayman Chemical; cat. no. 23125), doxorubicin (Selleck Chemicals; cat. no. S1208), gemcitabine (Selleck Chemicals; cat. no. S1714), olaparib (Selleck Chemicals; cat. no. S1060), and CGS-15943 (Tocris; cat. no. 1699).

### Other Genetic Manipulations

Generation of Control Clones: Rosa26 gRNA Clones

A CRISPR guide was designed to target the noncoding Rosa26 locus and cloned into the Lenti-Cas9-gRNA-GFP vector (Addgene #124770) as described above. Cells were transfected with the Rosa26 targeting gRNA CRISPR plasmid using Lipofectamine 3000, and GFP+ cells were single cell sorted onto 96-well plates. Clonal cell lines were established and subject to karyotypic validation through SMASH karyotyping prior to phenotypic characterization.

1q, 7p, and 8q gRNA clones: Cells were transfected with centromere-targeting gRNA CRISPR plasmids using Lipofectamine 3000 and clonal cell lines were established as described above for ReDACT-CO (Table 4A). Clones that maintained an extra copy of the targeted chromosome, as revealed by TaqMan copy number assays and SMASH-Seq, were used as control clones.

Olfactory gene deletions: CRISPR guides were designed to flank the coding sequence of the targeted olfactory gene and cloned into the Lenti-Cas9-gRNA-GFP vector (Addgene #124770) as described above. Cells were transfected with the olfactory gene deletion guides using Lipofectamine 3000, and single GFP+ cells were sorted onto 96-well plates. Clonal cell lines were established and screened for single copy deletions of the targeted olfactory genes with TaqMan copy number assays as described above. To exclude the possibility of a chromosome truncation event, additional TaqMan copy number assays were performed with probes telomeric to the targeted gene. Clones with single copy deletions of the targeted olfactory gene were subject to karyotypic validation through SMASH-Seq prior to phenotypic characterization.

Generation of Cassette Deletion Clones: A2780 and AGS integrant clones were co-transfected with a centromere targeting gRNA and a cassette proximal gRNA using Lipofectamine 3000. Cassette deletion was selected for with 10 μM ganciclovir (Sigma Aldrich, cat. no. G2536). Following ganciclovir selection, dsRed or GFP negative cells were single cell sorted onto 96-well plates, and clonal cell lines were established. Clones were screened for cassette loss with PCR primers spanning the deletion region (Table 5) and cassette deletion independent of any other karyotypic alterations was confirmed through SMASH karyotyping.

### Deletion of a Single Copy of MDM4 or UCK2

CRISPR guides were designed to flank the coding sequence of the targeted gene and cloned into the Lenti-Cas9-gRNA-GFP vector (Addgene #124770) as described above. For dual guide plasmids, dual guide gene blocks were ordered from IDT and cloned into the Lenti-Cas9-gRNA-GFP vector using NEBuilder HiFi DNA Assembly (New England Biolabs, cat. no. E2621L). CRISPR gRNA sequences are listed in Table 4A. Cells were transfected with the gene deletion guides using Lipofectamine 3000, and GFP+ cells were single cell sorted onto 96-well plates. Clonal cell lines were established and screened for single copy deletions of targeted genes with TaqMan copy number assays as described above. To exclude the possibility of a chromosome truncation event, additional TaqMan copy number assays were performed with probes telomeric to the targeted gene. Clones with single copy deletions of the targeted genes were subject to karyotypic validation through SMASH karyotyping prior to phenotypic characterization.

Transformation of MCF10A with HRASG12V

pBABE-HRASV12-Hyg (Addgene #195143) was generated by cloning hRASV12 from pBABE-HRASV12-puro (Addgene #9051) into pBABE-hyg (Addgene #1765) by digestion of both plasmids with BamHI and SalI, gel purification of digested DNA, ligation, and transformation into Stbl3 E. coli. The plasmid sequence was verified by sequencing. Viral preparation and transduction were performed as described above.

### Derivation of TP53-Knockout Clones

TP53-targeting gRNAs were designed and cloned into the Lenti-Cas9-gRNA-GFP vector (Addgene #124770) for generating TP53-KO clones. In parallel, an AAVS1-targeting gRNA was utilized to generate isogenic TP53-WT control clones. A2780 cells were transfected with these CRISPR gRNA plasmids and GFP+ cells were single cell sorted onto 96-well plates. Clonal cell lines were established and screened for TP53-KO through western blotting. Clones were subject to karyotypic validation through SMASH karyotyping prior to phenotypic characterization.

### Generation of HAP1 UCK2-KO Cells

HAP1 cells harboring a CRISPR-induced frameshift mutation in the UCK2 gene were purchased from Horizon Discovery (cat. no. HZGHC007067c006). Loss of UCK2 expression was verified by western blotting.

### Generation of A2780 Cells Constitutively Overexpressing Chromosome 1q Genes

A2780 cells were transduced with pLV-Bsd-CMV-hUCK (Addgene #195141), pLV-Bsd-CMV-MDM4 (Addgene #202669), pLV-Neo-CMV-MCL1 (Addgene #202668), or pLV-Hyg-CMV-BCL9 (Addgene #202667) and selected with 4 μg/mL blasticidin (InvivoGen; cat. no. ant-bl-1), 200 μg/ml G418 (InvivoGen: cat. no. ant-gn-1), or 100 μg/ml hygromycin (InvivoGen: cat. no. ant-hg-1). The plasmid sequences were verified by sequencing. Viral preparation and transduction were performed as described above.

### Sample Sizes and Statistical Techniques

For all soft agar experiments, the data displayed represent colony counts from at least 15 independent fields of view. All soft agar experiments were conducted at least twice (two or more independent biological replicates), and representative experiments are shown. Boxplots for soft agar assays display the 25th, 50th, and 75th percentiles of colonies per field, and the whiskers display the 10th and 90th percentiles.

For all drug sensitivity experiments, cells were plated in duplicate or triplicate and counted twice. All drug sensitivity experiments were performed at least three times (three or more independent biological replicates), with the exception of the control experiments, which were performed once. Bar graphs in drug sensitivity assays display the means±SEM.

For the proliferation experiments, two independent biological replicates were performed, and the average of both replicates are displayed.

For the competition experiment, two independent experiments were performed: one when cells were mixed at an 80-20 ratio and one when cells were mixed at a 90:10 ratio. Similar results were obtained in both experiments.

The number of mice used in each xenograft experiment are as follows: 2E: n=10 mice for A2058 xenografts; n=5 mice for A2780 1q-loss c1 xenografts; n=6 mice for A2780 1q-loss c2 xenografts. 2H: n=10 mice for 1q-loss c2 xenografts; n=9 mice for 1q-loss c3 xenografts. 3D: n=8 mice for 7p-loss c1 xenografts; n=15 mice for 7p-loss c2 and 8q-loss c1 xenografts; n=9 mice for 8q-loss c2 xenografts. 3H: n=9 mice for each experiment. 4A: n=8 mice. 4D: n=9 mice. 4E: n=10 mice. 4G: n=9 mice. S5A: n=10 mice. S5B: n=10 mice. S5C: n=10 mice. S11A: n=7 mice. Tumor growth curves in xenograft assays display the means±SEM.

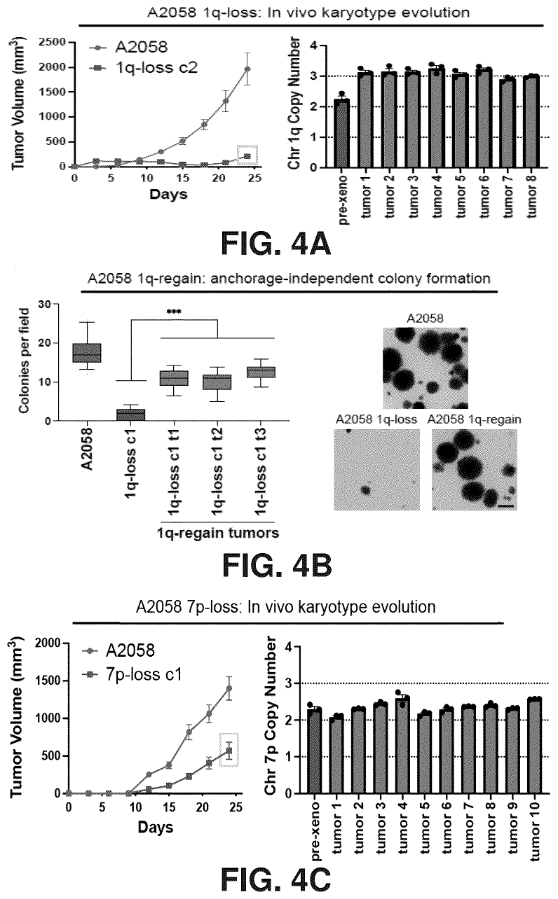

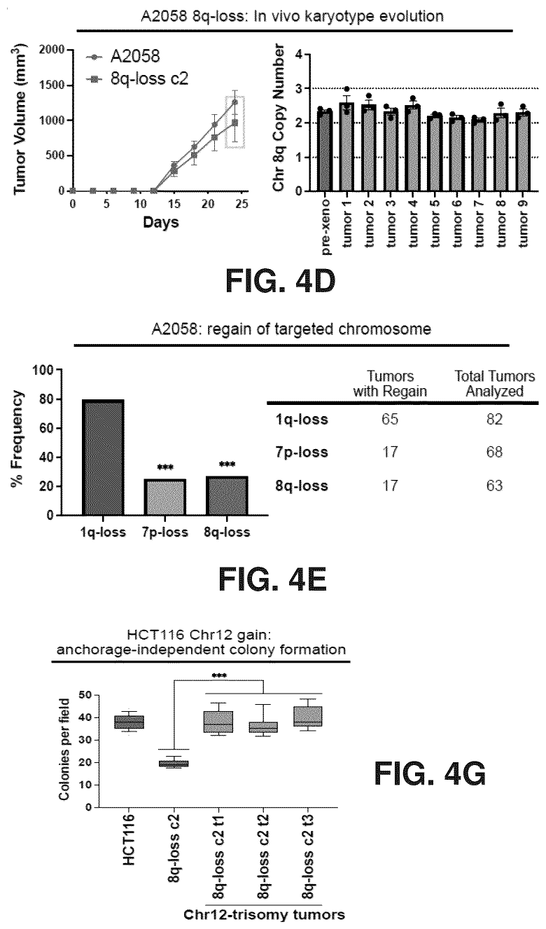

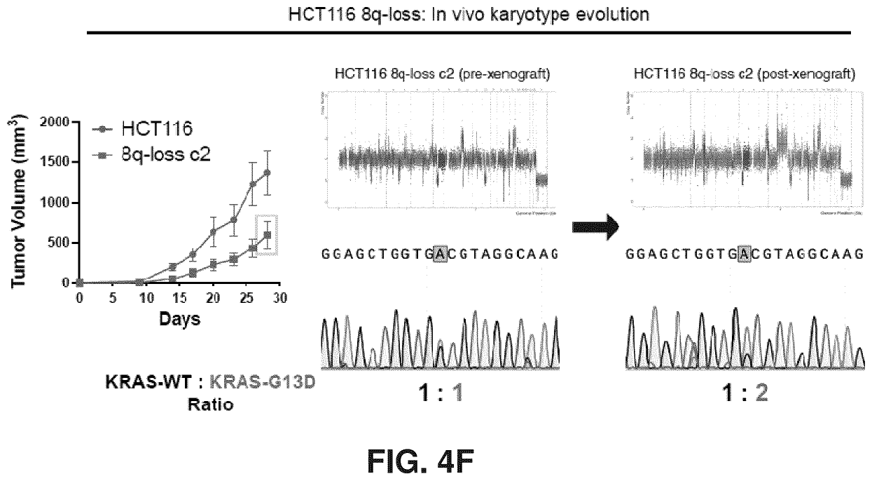

Data in most figures were analyzed by unpaired t-tests in the following figures: Data were analyzed by chi-square tests in FIG. 4E.

### Computational Approaches: Copy Number Timing Analysis

Raw whole genome sequencing (WGS) data in Barn or Fastq formats were downloaded from public databases provided by the original publications (Yates, et al., Cancer Cell. 32, 169-184.e7 (2017), Paterson, Proc. Natl. Acad. Sci. 117, 20681-20688 (2020), Kawazu, et al., PLoS Genet. 13, e1006853 (2017)). ith.Variant pipeline (available at world wide web address: “//github.com/SunPathLab/ith.Variant”) was used to call somatic copy number alterations (SCNA) and point mutations and to determine the clonality of these somatic variants (Maeser, et al., STAR Protoc. 4, 101927 (2023)). In total, 38 breast cancer samples (BRCA) from 21 patients and 37 melanoma (MEL) primary tumor samples (all paired with normal control samples) passed the WGS data quality control and were included in the analysis. To reconstruct the evolutionary history of SCNAs in patient tumors, BUTTE was applied to infer the initiation time of clonal CN gains. The initiation time was defined as the time fraction when the first gain occurs for a clonal SCNA. In brief, BUTTE estimates the initiation time of complex gains (or gains involving multiple steps) by modeling the quantitative relationship between point mutations and paths of copy number events. To do so, it first adopts the expectation-maximization (EM) algorithm to find the allele state distribution of point mutations. BUTTE then either directly solves the timing for SCNAs with identifiable CN history matrices (Purdom, et al., Bioinforma. Oxf Engl. 29, 3113-3120 (2013)) or adopts linear programming to calculate the upper bounds of the initiation time if the underlying linear system is underdetermined (multiple history matrices exist for an SCNA)(Wang, et al., Evolving copy number gains promote tumor expansion and bolster mutational diversification (2022)). Clustered gains were identified by clustering the inferred timing via nonparametric density estimation (Azzalini & Menardi, J. Stat. Softw. 57, 1-26 (2014)). Genome doubling was identified as the cluster containing more than 40% of the segments. The accession numbers for the datasets are as follows: JGAD000095, EGAD00001002696, and EGAD00001003388.

### Detecting Recurrent Early Gains

To identify genomic regions exhibiting recurrent early gains across patients, the hg38 genome was scanned with bins of 1 million bp in size and ranked the bins in each sample according to the timing of the respective initiating gain. The timing values were jittered to avoid ties. The middle rank of the respective sample was then subtracted from each bin. The middle rank was the expectation value of the ranks under the assumption that the null hypothesis holds: no regions show recurrent early gain across patients. For each tumor type, the resulting ranks were summed across patients for each bin. Division of the rank sums by its standard deviation yielded normalized rank sums which approximately follow a standard normal distribution if the null hypothesis is fulfilled. A large negative normalized rank sum would reject the null hypothesis and indicate recurrent early initiating gains. To evaluate the frequency of gains for each genomic bin across patients, the segment mean (read depth ratio between the tumor and normal samples) were ranked as done for the timing values, and then the same rank sum normalization was performed. A large positive normalized rank sum for the segment mean would suggest frequent gains across patients.

### Analysis of Mutual Exclusivity Between Aneuploidy and Oncogenic Mutations

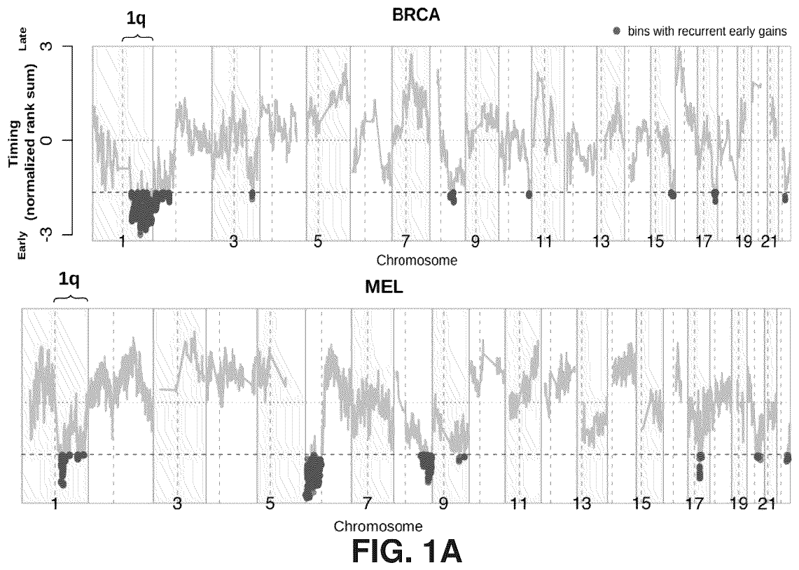

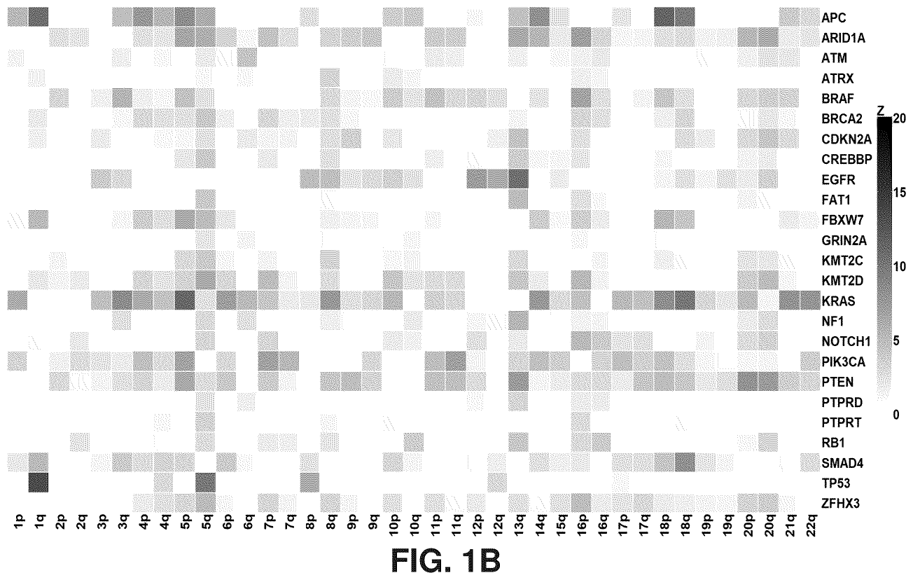

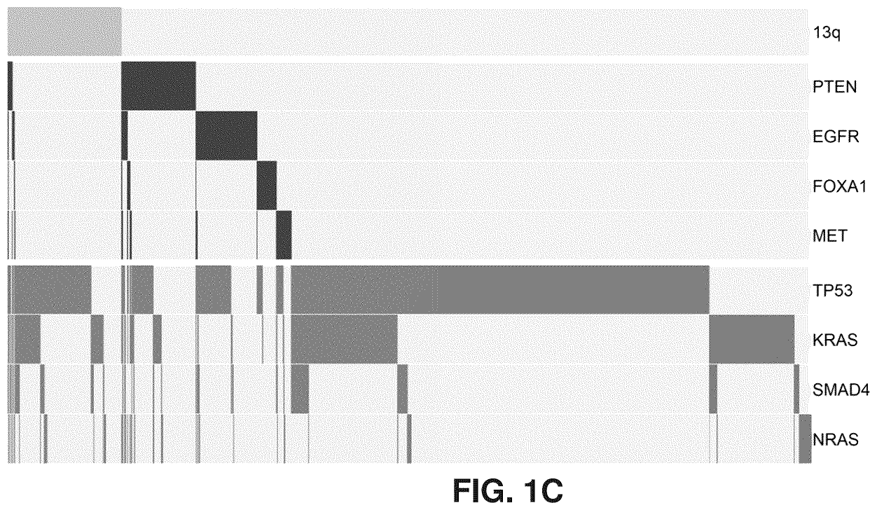

To analyze the mutual exclusivity between aneuploidy and oncogenic mutation, the MSK-MET (Nguyen, et al., Cell. 185, 563-575.ell (2022)) dataset available via the public cBioPortal datahub was used as a starting point. In order for a given chromosomal arm and oncogene to be compared, the data were required to pass a set of quality filters. All cancer types were required to have >25 patients and selected a representative sample for each patient based on available mutation and arm gain data. sets of genes and arms were built by cancer type by applying additional requirements by cancer. The sets of genes that were analyzed started from the IMPACT-505 geneset and each gene was required to be mutated in >2% of patients by cancer type. The sets of arms that were analyzed started from all previously generated arm gain data by sample and each arm was required to exceed a minimum of 100 patients or >2% of patients experiencing arm gain by cancer type. Once sets of genes and arms by cancer were cleaned, contingency tables were calculated for each combination and conducted a two-sided Fisher's exact test to compute p-values and odds ratios for each gene X arm combination. These p-values were converted into Z scores for further comparisons and applied the sign of the negative log 2 of the odds ratio to represent the direction of the relationship; therefore, a large positive Z would suggest significant mutual exclusivity, while a large negative Z would suggest significant co-occurrence. This process was repeated this in a pancancer analysis by utilizing the same cleaned data and pooling all patients, genes, and arms into a single cohort. All mutual exclusivity analysis was automated using Nextflow (Di Tommaso, et al., Nat. Biotechnol. 35, 316-319 (2017)) and Conda (Anaconda—The World's Most Popular Data Science Platform. Anaconda, (available at anaconda.com/)), starting from downloading MSK-MET data through to generation of FIGS. 1D-E and Tables 3A-3B.

### Analysis of Copy Number Alterations and Patient Survival

To examine the relationship between copy number gains, mutations, and cancer patient outcomes in TCGA, univariate Cox proportional hazards models were used as described in refs (Smith & Sheltzer, eLife. 7, e39217 (2018), Smith & Sheltzer, Cell Rep. 38, 110569 (2022)). In brief, TCGA copy number data (broad.mit.edu_PANCAN_Genome_Wide_SNP_6_whitelisted.seg, available at world wide web address: “//gdc.cancer.gov/about-data/publications/pancanatlas”) and TCGA mutation data (mc3.v0.2.8.PUBLIC.maf.gz, available at world wide web address: “//gdc.cancer.gov/about-data/publications/pancanatlas”) were combined with patient outcomes data described in ref (Liu, et al., Cell. 173, 400-416.ell (2018)). Selection of the clinical endpoint for each cancer type was based on the recommendations provided by ref (Liu, et al., Cell. 173, 400-416.ell (2018)) based on data quality, cohort size, and the number of events that were observed. TCGA copy number data was generated as relative copy number values for particular chromosomal intervals. This data was translated to produce a single copy number value on a per-gene basis, based on the observed copy number at each gene's transcription start site. This annotation was performed using mapping data from GENCODE v32. Z scores for copy number changes and for mutations in common cancer driver genes were calculated using Cox proportional hazards regression. For the copy number analysis, genes were mapped back to chromosome bands, and the top-scoring gene (based on the pan-cancer Stouffer's Z score) was identified for each band. For the mutation analysis, a gene was considered to be mutated if there was a single non-synonymous mutation at any codon within the gene. Non-synonymous mutations included: missense, nonsense, frameshift deletion, splice site, frameshift insertion, in-frame deletion, translation start site, nonstop mutation, and in-frame insertion. Additional clinical data were downloaded from cBioportal.

### Analysis of Aneuploidy-Associated Gene Expression

Chromosome copy number data for cancers from TCGA were downloaded from ref (Taylor, et al., Cancer Cell. 33, 676-689.e3 (2018)). Chromosome copy number data for the Cancer Cell Line Encyclopedia was downloaded from ref (Cohen-Sharir, et al., Nature. 590, 486-491 (2021)). Gene expression data for cancers from TCGA was downloaded from the TCGA PanCanAtlas (EBPlusPlusAdjustPANCAN_IlluminaHiSeq_RNASeqV2.geneExp.tsv (available at world wide web address: “//gdc.cancer.gov/about-data/publications/pancanatlas”). Gene expression data for the Cancer Cell Line Encyclopedia was downloaded from DepMap (available at world wide web address: “depmap.org”).

### Data Visualization

Scientific illustrations were generated with Biorender. Most graphs were generated using GraphPad Prism. Boxplots display the 25th, 50th, and 75th percentiles of colonies per field, while the whiskers represent the 10th and 90th percentiles. Unless otherwise indicated, bar graphs and XY plots display the means±SEM.

### Results

### Specific Chromosome Gains Recurrently Occur Early in Cancer Development

A computational approach to leverage multi-sample tumor sequencing data was established to determine the relative timing of somatic copy number alterations (SCNAs) in cancer evolution (Wang, et al., Evolving copy number gains promote tumor expansion and bolster mutational diversification (2022)). This tool was applied to investigate the timing of aneuploidy events in a cohort of patients with breast cancer (BRCA) or melanoma (MEL)(Yates, et al., Cancer Cell. 32, 169-184.e7 (2017), Hayward, et al., Nature. 545, 175-180 (2017)).

Relative timing of SCNAs in whole genome sequences from these tumor samples were modeled by assuming that:
(1) somatic point mutations accumulate over time at a rate that is proportional to DNA copy number; and
(2) the multiplicity of early point mutations increases with copy number gains.

It was found that specific chromosome copy number changes are consistently observed early in tumor development (FIGS. 1A-1B).

It was observed that chromosome 1q gains are recurrently the first copy number alterations that occur in breast cancer evolution, and these gains are also among the first alterations in melanoma evolution. In general, it was found that common aneuploidies arose earlier in tumor development than less-common aneuploidies, in agreement with the assumption that early somatic alterations are likely to be fitness-driving events (Paterson, Proc. Natl. Acad. Sci. 117, 20681-20688 (2020)). However, the correlation between frequency and timing was not maintained across all chromosomes. For instance, in breast cancer, chromosome 8q gains and chromosome 1q gains occurred with similar frequencies, but it was found that 1q gains consistently arose earlier during tumor development than 8q gains. As previously observed with oncogenic point mutations (McGranahan, et al., Sci. Transl. Med. 7, (2015)), specific chromosome gains occur in a defined temporal order, and aneuploidies that are consistently gained early during tumorigenesis may enhance cancer fitness.

Specific Chromosome Gains are Associated with Altered Mutational Patterns and Cancer Progression

In instances where two oncogenes converge to activate the same pathway, cancers frequently acquire mutations in either of the genes, but not both (Cisowski, et al., Small GTPases. 8, 187-192 (2017)). If chromosome gains play an oncogene-like role supporting cancer growth, then specific aneuploidies may also exhibit mutual exclusivity with individual oncogenic mutations. To investigate this possibility, patterns of mutual exclusivity between chromosome arm gains and mutations were calculated across 23,544 cancer patients (Nguyen, et al., Cell. 185, 563-575.el1 (2022), Hagenson, Sheltzer-lab/aneuploidy-addictions: v1.0.0 (2023)). Several hundred instances in which aneuploidies and mutations co-occur less often than expected were detected by chance both within individual cancer types and in a pan-cancer analysis (FIGS. 1C-1D), and Tables 3A-3B. For instance, KRAS mutations are mutually exclusive with chromosome 18q gains in pancreatic cancer (Table 3A), while BRAF mutations are mutually exclusive with chromosome 20q gains in colorectal cancer (Table 3B). These results are consistent with the hypothesis that specific chromosome gains can play an oncogene-like role in cancer, thereby making the acquisition of certain oncogenic mutations redundant in the presence of that aneuploidy.

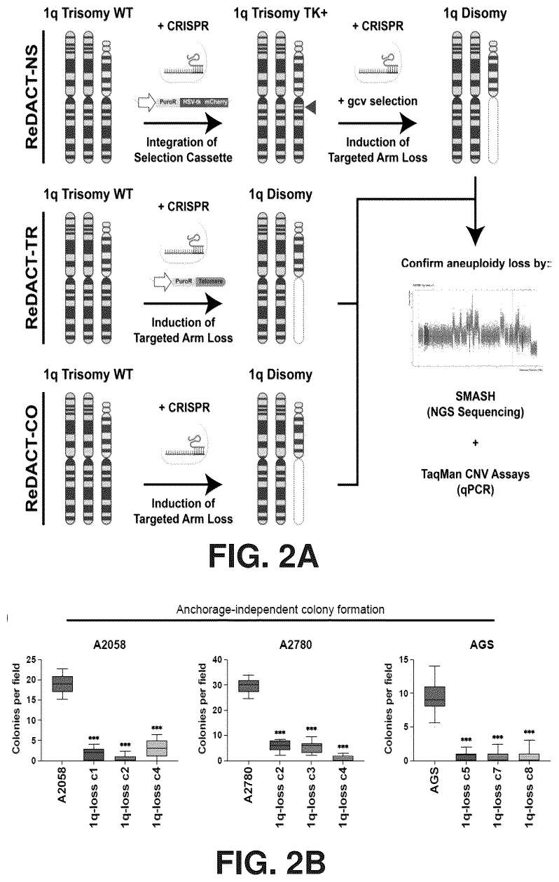

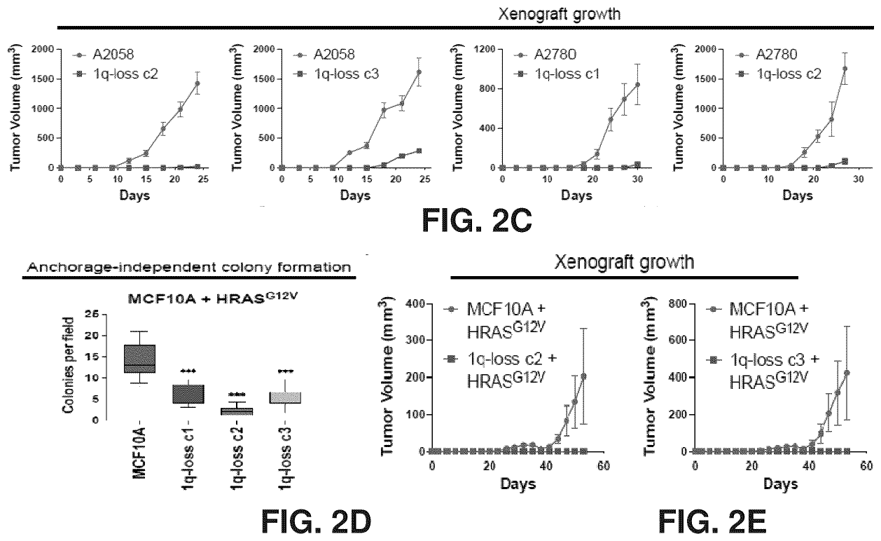

High levels of aneuploidy are generally associated with poor cancer patient outcomes (Stopsack, et al., Proc. Natl. Acad. Sci. 116, 11390-11395 (2019), Hieronymus, et al., eLife. 7, e37294 (2018), Lukow, et al., Dev. Cell. 56, 2427-2439.e4 (2021)). However, it is less clear whether specific copy number changes drive tumor progression, or whether the aneuploid state itself represents a universal risk factor. the association between patient outcome and copy number gains affecting every chromosome band were calculated across 10,884 patients and 33 cancer types from The Cancer Genome Atlas (TCGA)(Smith, Genome-wide identification and analysis of prognostic features in human cancers (2022)). It was determined that certain copy number alterations were commonly prognostic across multiple cancer types, particularly gains affecting chromosome 1q (FIG. 2A). The strong association between 1q gains and disease progression was robust to the inclusion of multiple clinical variables, including patient age, sex, tumor stage, and tumor grade. 1q copy number correlated with hallmarks of aggressive disease in genetically-diverse cancer types, including with Gleason score in prostate adenocarcinoma and with thrombocytopenia in acute myeloid leukemia.

A similar analysis was performed for cancer-associated mutations, and it was found that the only gene for which mutations were prognostic in more than four cancer types was TP53. These results illustrate that specific chromosome gain events, particularly involving regions of chromosome 1q, are robust pan-cancer markers for the risk of disease progression.

TABLE 3A
Pancreatic cancer

| Gene | 18q |
|---|---|
| ARID1A | 0.20706246 |
| ATM | −0.34383414 |
| ATRX | −10.5886883 |
| BRCA2 | 0 |
| CDKN2A | 4.49348983 |
| DAXX | −9.18184836 |
| GNAS | 0 |
| KDM6A | 0.90877917 |
| KMT2C | 0 |
| KMT2D | 1.36368 |
| KRAS | 13.8590105 |
| MEN1 | −14.5779463 |
| PIK3CA | 1.11435991 |
| PTPRT | 0 |
| RB1 | −0.79204316 |
| RBM10 | 0.35834459 |
| RNF43 | 0.2590655 |
| SETD2 | −4.91361137 |
| SMAD4 | 5.42034829 |
| TGFBR1 | 1.12898269 |
| TGFBR2 | 1.56806174 |
| TP53 | 8.41199883 |
| TSC2 | −6.42793718 |

TABLE 3B
Colorectal cancer

| Gene | 20q |
|---|---|
| ABL1 | 5.666204577 |
| ALK | 3.658353937 |
| AMER1 | 3.585952119 |
| ANKRD11 | 6.340742646 |
| APC | −10.824061 |
| AR | 4.50570215 |
| ARID1A | 8.433996164 |
| ARID1B | 6.280679971 |
| ARID2 | 5.638525717 |
| ARID5B | 5.166788991 |
| ASXL1 | 4.888737554 |
| ASXL2 | 4.224025531 |
| ATM | 5.927443273 |
| ATR | 5.345565333 |
| ATRX | 3.613495368 |
| AXIN1 | 5.821333657 |
| AXIN2 | 5.521772927 |
| AXL | 6.005244389 |
| B2M | 8.754659164 |
| BCOR | 6.785194087 |
| BLM | 4.654167525 |
| BRAF | 8.777224838 |
| BRCA1 | 5.442994138 |

### Loss of Trisomy 1q Blocks Malignant Growth in Human Cancers

The computational analyses described above highlighted several similarities between chromosome copy number gains and driver mutations, raising the possibility that these aneuploidies could represent oncogene-like cancer addictions. The oncogene addiction paradigm was first established by developing genetic techniques to eliminate individual genes from established cancer cell lines (Shirasawa, et al., Science. 260, 85-88 (1993), Weinstein, Science. 297, 63-64 (2002)). In order to conduct comparable assays with aneuploidy, a set of approaches collectively called ReDACT (Restoring Disomy in Aneuploid cells using CRISPR Targeting) was created (FIG. 2A).

In the first approach, called ReDACT-NS (Negative Selection), a copy of herpesvirus thymidine kinase (HSV-TK) is integrated onto an aneuploid chromosome of interest. Then, the cells are transfected with a gRNA that cuts between the integrant and the centromere and treated with ganciclovir, which is toxic to cells that express HSV-TK (Fillat, et al., Curr. Gene Ther. 3, 13-26 (2003)). Loss of the aneuploid chromosome harboring HSV-TK allows cells to survive ganciclovir selection.

In the second approach, called ReDACT-TR (Telomere Replacement), cells are co-transfected with a gRNA that cuts near the centromere of an aneuploid chromosome along with a cassette encoding ˜100 repeats of the human telomere seed sequence (Uno, et al., Sci. Rep. 7, 12739 (2017)). CRISPR cleavage coupled with integration of the telomeric seed sequence leads to loss of an aneuploid chromosome arm and formation of a de novo telomere.

In the third approach, called ReDACT-CO (CRISPR Only), prior reports demonstrating that in rare circumstances CRISPR cleavage by itself is sufficient to trigger chromosome loss were considered (Zuccaro, et al., Cell. 183, 1650-1664.e15 (2020), Papathanasiou, et al., Nat. Commun. 12, 5855 (2021)), and cells were transfected with a gRNA targeting an aneuploid chromosome arm without any other selection markers.

All three approaches were successfully applied to create clones derived from human cell lines that had lost specific aneuploid chromosomes.

Aneuploidies of chromosome 1q were initially analyzed, as it was found that 1q gains were an early event in multiple cancer types and were strongly associated with disease progression (FIGS. 1A-1D). The 1q trisomy in the A2058 melanoma cell line, AGS gastric cancer cell line, and A2780 ovarian cancer cell line were targeted. Multiple independent derivatives of each line were generated in which a single copy of chromosome 1q had been eliminated, thereby producing cell lines that were disomic rather than trisomic for chromosome 1q. Loss of the 1q trisomy and the absence of any other chromosome copy number changes were verified using SMASH-Seq, a sequencing-based approach to determine DNA copy number (Wang, et al., Genome Res. 26, 844-851 (2016)), and by G-banding analysis of metaphase spreads (Table 2). Loss of the 1q trisomy decreased the expression of genes encoded on chromosome 1q by an average of 26% at the RNA level and 21% at the protein level. These results indicated that chromosome loss causes a substantial downregulation of genes encoded on an aneuploid chromosome, though these effects can be buffered to some extent by cellular dosage compensation (Schukken & Sheltzer, Genome Res. 32, 1254-1270 (2022)).

Next, whether losing the 1q trisomy affects malignant growth in cancer cells was tested. Toward that end, anchorage-independent colony formation, an in vitro proxy for malignant potential (Shin, Proc. Natl. Acad. Sci. 72, 4435-4439 (1975)), was quantified in the 1q-trisomic and 1q-disomic cells. While 1q-trisomic A2058, A2780, and AGS cells displayed robust colony formation, multiple independent 1q-disomic clones derived from each cell line exhibited minimal anchorage-independent growth (FIG. 2B).

Contralateral subcutaneous injections were then performed with each cell line to test whether aneuploidy-loss affected tumor growth in vivo. Consistent with the colony formation assays, it was observed that 1q-trisomic A2058 and A2780 cells rapidly formed large tumors, while 1q-disomic cells displayed minimal tumor growth (FIG. 2C). At the end of these assays, the trisomic cells had formed tumors that were on average 25-fold larger than the tumors formed by the 1q-disomic cells. For the AGS cancer cell line, neither the trisomic nor the disomic cells formed tumors following subcutaneous injection. Finally, proliferation assays were performed to measure the doubling time of the 1q-trisomic and the 1q-disomic cells in culture. The aneuploidy-loss clones divided more slowly in vitro compared to the 1q-trisomic cells, though the difference in doubling time (˜35%) was substantially less than the differences observed in the soft agar and xenograft assays.

Taken together, these results indicate that multiple human cancer cell lines are dependent on the presence of a third copy of chromosome 1q to support malignant growth, and elimination of this aneuploid chromosome compromises their tumorigenic potential. Furthermore, this phenotypic pattern, in which aneuploidy loss causes a moderate effect on in vitro doubling but a severe effect on anchorage-independent growth and xenograft formation, resembles the previously-reported consequences of eliminating bona fide oncogene addictions (Shirasawa, et al., Science. 260, 85-88 (1993), Chin, et al., Nature. 400, 468-472 (1999)).

### Loss of Trisomy 1q Prevents Malignant Transformation

1q gains were commonly the first copy number alteration to occur during breast tumor development (FIGS. 1A-1B). It was hypothesized that, in addition to being required for cancer growth, aneuploidy of chromosome 1q may directly promote cellular transformation. To test this, chromosome engineering was performed in MCF10A, an immortal but non-tumorigenic mammary epithelial cell line. SMASH-Seq revealed that this cell line harbors a trisomy of chromosome 1q, and ReDACT-CO and ReDACT-TR were successfully applied to generate derivatives of MCF10A with two copies rather than three copies of 1q (Table 2). Transformation of the 1q-trisomic and 1q-disomic cells was then attempted by transducing them with a retrovirus encoding the HRASG12V oncogene.

HRASG12V expression was sufficient to transform trisomic MCF10A, as these cells were able to form colonies in soft agar and grow as xenografts in nude mice (FIGS. 2D-2E). In contrast, 1q-disomic MCF10A clones expressing HRASG12V exhibited impaired colony formation and were unable to produce tumors in vivo, demonstrating that loss of the trisomic chromosome prevented cellular transformation. These results are consistent with the finding that 1q gains are an early event during breast cancer development and demonstrate that specific aneuploidies can cooperate with oncogenes to transform non-malignant cells.

### Robust Anchorage-Independent Growth in Human Cancer Cell Lines Subjected to CRISPR Cutting and Ganciclovir Selection

In order to confirm that loss of cellular transformation was a specific consequence of aneuploidy loss, a series of control clones subjected to various CRISPR manipulations that did not induce loss of the 1q trisomy were generated and tested. These control clones included:
1) Cell lines harboring a CRISPR-mediated integration of the HSV-TK cassette that were not subjected to selection for 1q-loss;
2) Cell lines in which the HSV-TK cassette was deleted using two gRNAs followed by ganciclovir selection;
3) Cell lines transfected with a 1q-targeting gRNA in which the genetic cleavage site was repaired without inducing chromosome loss;
4) Cell lines transfected with a gRNA targeting the non-coding Rosa26 locus;
5) Cell lines in which dual CRISPR guides were used to generate segmental deletions of genes encoding olfactory receptors on chromosome 1q; and
6) Cell lines in which CRISPR was used to delete a terminal segment on chromosome 1q, eliminating the telomere and decreasing the copy number of 26 out of 968 protein-coding genes on the chromosome.

SMASH-Seq was performed on each control clone generated and it was confirmed that each clone retained an extra copy of chromosome 1q. The effects of these manipulations on anchorage-independent growth were then measured. It was found that every control clone maintained the ability to form colonies in soft agar, with some variability between independent clones. Across the three cancer cell lines and 37 different control clones, it was observed that the 1q-disomic clones exhibited worse anchorage-independent growth than every control clone that was generated. These results indicate that the deficiencies in malignant growth exhibited by the 1q-disomic clones are not a result of the use of CRISPR or ganciclovir selection, per se.

### Eliminating Different Cancer Aneuploidies Produces Distinct Phenotypic Consequences

To further investigate the consequences of inducing aneuploidy loss, ReDACT was used to eliminate the trisomy of either chromosome 7p or 8q from A2058 melanoma cells. SMASH-Seq confirmed the desired aneuploidy-loss events without other karyotypic changes (Table 2).

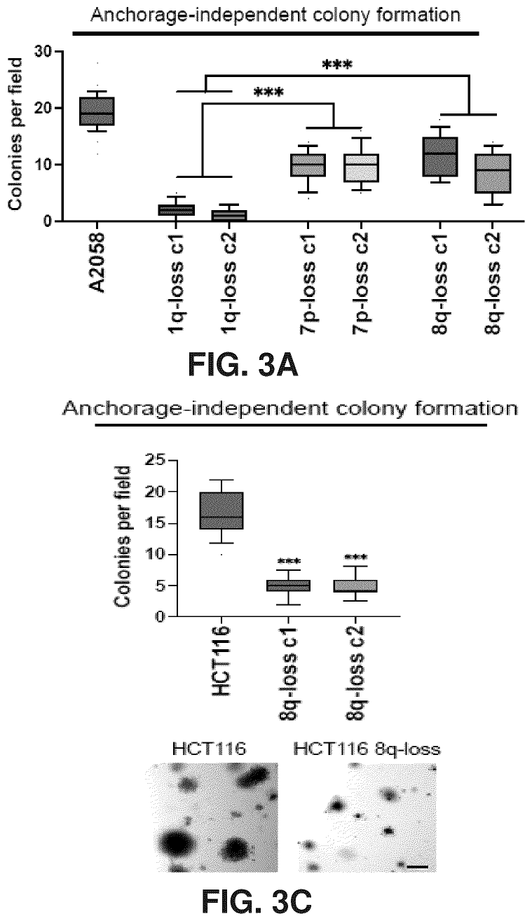

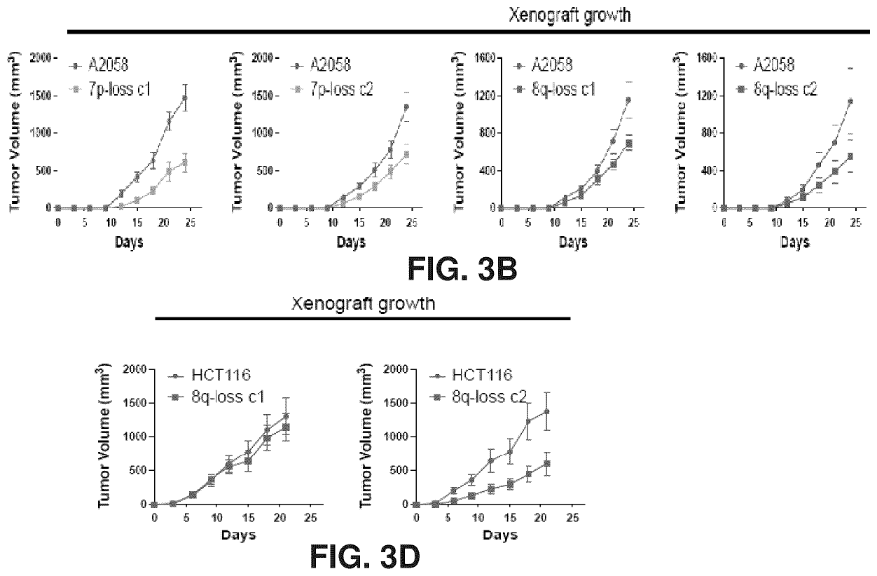

Loss of either trisomy 7p or trisomy 8q resulted in a decrease in the expression of genes encoded on the affected chromosomes. 7p-disomic and 8q-disomic clones exhibited impaired anchorage-independent growth compared to a panel of control clones, though this defect was not as severe as the defect observed among A2058 1q-disomic clones (FIG. 3A).

In vitro doubling times were also closer to wild-type levels for 7p-disomic and 8q-disomic cells compared to 1q-disomic cells. Finally, subcutaneous injections of the 7p-disomic and the 8q-disomic cells were performed in nude mice, and it was found that loss of either the 7p or the 8q trisomy resulted in a moderate decrease in tumor growth (FIG. 3B). At the end of the assay, the wild-type tumors were on average two-fold larger than the tumors formed by either 7p-disomic or 8q-disomic cells, compared to a 30-fold difference between A2058 wild-type and 1q-disomic tumors (FIG. 2C). In total, these results indicate that A2058 melanoma cells exhibit a greater degree of “addiction” to the 1q trisomy compared to the trisomies of chromosome 7p or 8q.

To explore the consequences of losing chromosome 8q aneuploidy in a distinct cancer type, the 8q trisomy from the colorectal cancer cell line HCT116 was eliminated (Table 2).

TABLE 2
Diploid-like cell-lines prepared from parental aneuploid cell lines

| Parental cell line | Clone name | Method |
|---|---|---|
| A2058 | 1q-loss c1 | ReDACT-CO |
| A2058 | 1q-loss c2 | ReDACT-CO |
| A2058 | 1q-loss c3 | ReDACT-CO |
| A2058 | 1q-loss c4 | ReDACT-CO |
| A2780 | 1q-loss c1 | ReDACT-NS |
| A2780 | 1q-loss c2 | ReDACT-NS |
| A2780 | 1q-loss c3 | ReDACT-NS |
| A2780 | 1q-loss c4 | ReDACT-NS |
| A2780 | 1q-loss c5 | ReDACT-NS |
| A2780 | 1q-loss c6 | ReDACT-NS |
| A2780 | 1q-loss c8 | ReDACT-NS |
| AGS | 1q-loss c1 | ReDACT-NS |
| AGS | 1q-loss c4 | ReDACT-NS |
| AGS | 1q-loss c5 | ReDACT-NS |
| AGS | 1q-loss c6 | ReDACT-NS |
| AGS | 1q-loss c7 | ReDACT-NS |
| AGS | 1q-loss c8 | ReDACT-NS |
| AGS | 1q-loss c11 | ReDACT-TR |
| AGS | 1q-loss c12 | ReDACT-TR |
| MCF10A | 1q-loss c1 | ReDACT-TR |
| MCF10A | 1q-loss c2 | ReDACT-TR |
| MCF10A | 1q-loss c3 | ReDACT-TR |
| MCF10A | 1q-loss c4 | ReDACT-CO |
| A2058 | 7p-loss c1 | ReDACT-CO |
| A2058 | 7p-loss c2 | ReDACT-CO |
| A2058 | 7p-loss c3 | ReDACT-CO |
| A2058 | 8q-loss c1 | ReDACT-CO |
| A2058 | 8q-loss c2 | ReDACT-CO |
| HCT116 | 8q-loss c1 | ReDACT-CO |
| HCT116 | 8q-loss c2 | ReDACT-CO |

Consistent with the observations in A2058, loss of the 8q trisomy decreased but did not fully prevent anchorage-independent growth in HCT116 (FIG. 3C). Xenograft formation was then tested in the HCT116 8q-disomic cells, and it was observed that one 8q-disomic clone exhibited a moderate defect in tumor growth while a second clone was able to form tumors at levels comparable to the trisomic parental line (FIG. 3D).

These results demonstrate that eliminating aneuploid chromosomes has variable effects, depending on the identity of the chromosome and the genetic background of the cancer.

Karyotype Evolution and 1q Trisomy Restoration after Aneuploidy Loss

A hallmark of oncogene addictions is that loss or inhibition of a driver oncogene results in strong and rapid selection to re-establish oncogenic signaling (Torti & Trusolino, EMBO Mol. Med. 3, 623-636 (2011)). For instance, when EGFR-driven lung cancers are treated with an EGFR inhibitor, these cancers evolve to acquire specific mutations, including EGFRT790M (which restores EGFR activity) and KRASG13D (which activates a parallel oncogenic pathway)(Westover, et al., Ann. Oncol. 29, i10-i19 (2018)).

Whether elimination of an “aneuploidy addiction” would also result in evolutionary pressure to restore the lost aneuploidy was investigated.

1q-disomic A2058 cells were injected into nude mice and then the copy number of chromosome 1q in the resulting xenografts were determined using qPCR (FIG. 4A). It was discovered that 65 out of 82 1q-disomic xenografts re-acquired an extra copy of chromosome 1q, demonstrating strong selective pressure to regain the initial 1q aneuploidy. 20 of these post-xenograft clones were subjected to SMASH-Seq, and it was found that chromosome 1q-regain was the only detectable chromosome-scale copy number change. No gross karyotypic changes were observed when the parental 1q-trisomic cells were grown as xenografts. If loss of the chromosome 1q trisomy compromises malignant potential, then it may be that regaining 1q aneuploidy would restore cell fitness. Consistent with this, it was found that cells that had re-acquired the 1q trisomy exhibited increased clonogenicity compared to 1q-disomic cells when grown under anchorage-independent conditions (FIG. 4B).

Next, karyotype evolution was assessed following in vivo growth of A2058 7p-disomic and 8q-disomic clones. Interestingly, 17 out of 68 7p-disomic xenografts and 17 out of 63 8q-disomic xenografts were found to exhibit 7p and 8q trisomy regain, respectively (FIGS. 4C-4D). These rates of chromosome re-gain were significantly lower than the rates that were observed for chromosome 1q (P<0.0001, chi-square test; FIG. 4E).

These results indicate that there is moderate selective pressure to restore 7p and 8q trisomies and stronger selective pressure to restore 1q trisomy in A2058.

Whether evolutionary pressure to restore chromosome 1q aneuploidy was present when 1q-disomic cells are grown in vitro was then assessed.

A2058, A2780, and AGS 1q-trisomic and 1q-disomic cancer cell lines were passaged for thirty days in culture and their karyotypes assessed. Similar to the in vivo results, multiple instances in which 1q-disomic cells independently regained an extra copy of chromosome 1q were uncovered over the course of the assay. It was reasoned that there were two possible sources for the regained chromosome:
(i) the third copy of 1q could result from the miss-segregation of an endogenous copy of chromosome 1q; or
(ii) the disomic cell line could have been out-competed by an exogenous population of trisomic cells (for instance, from metastatic colonization when 1q-disomic and 1q-trisomic xenografts were grown in the same mouse).

To differentiate between these possibilities, SNPs on 1q that were heterozygous in the parental cell line and that exhibited loss-of-heterozygosity (LOH) following 1q-elimination were identified. It was reasoned that if LOH was maintained following 1q-regain, that would be evidence for an endogenous miss-segregation event, while the reappearance of heterozygosity would be evidence of an exogenous cell population.

Sanger sequencing analysis revealed that both potential causes of 1q-regain were observed during these evolution experiments. In both cases, 1q-regain correlated with restored anchorage-independent growth relative to the 1q-disomic clones, further verifying the link between 1q copy number and cell fitness.

Finally, karyotype evolution in xenografts produced by 8q-disomic HCT116 cells was assessed (FIG. 4F).

It was found that 0 out of 13 tumors regained the trisomy of chromosome 8q, but 7 out of 13 tumors gained a de novo trisomy of chromosome 12. HCT116 cells are driven by a heterozygous KRASG13D mutation (Shirasawa, et al., Science. 260, 85-88 (1993)), and KRAS is encoded on chromosome 12. Sanger sequencing analysis revealed that every chromosome 12-trisomic tumor had amplified the copy of chromosome 12 harboring the mutant KRASG13D allele (FIG. 4G). Increasing dosage of mutant KRAS has previously been associated with enhanced tumor fitness (Burgess, et al., Cell. 168, 817-829.e15 (2017)), and it was observed that these chromosome 8q-disomic/chromosome 12-trisomic cells exhibited superior anchorage-independent growth relative to the chromosome 8q-disomic/chromosome 12-disomic pre-xenograft population (FIG. 4G).

Together, these results indicate that aneuploidy loss creates strong selective pressure for karyotype evolution, and the effects of aneuploidy loss can be suppressed in cis (by regaining the lost chromosome) or in trans (by acquiring a beneficial secondary alteration).

### Chromosome 1q Aneuploidy Suppresses p53 Signaling by Increasing MDM4 Expression

The biological mechanism underlying the addiction to chromosome 1q aneuploidy were investigated.

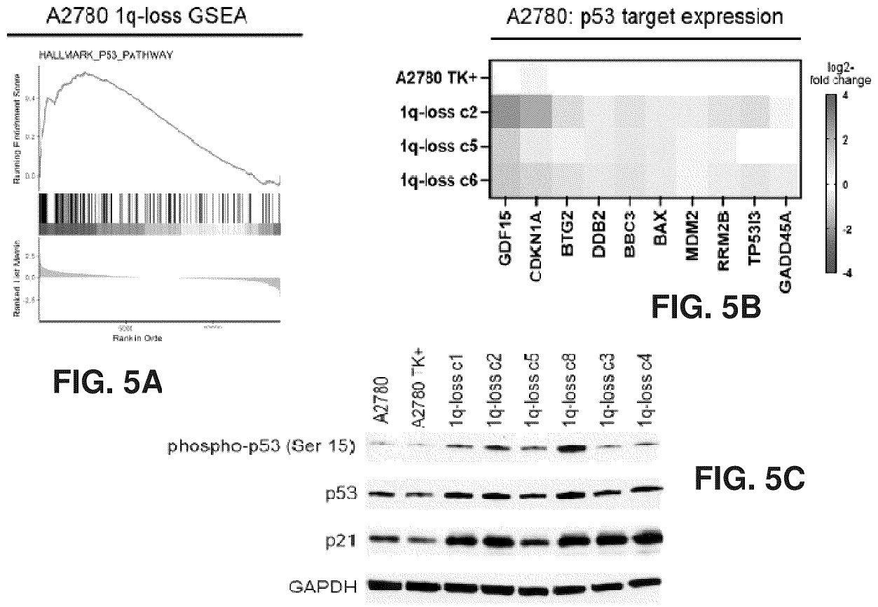

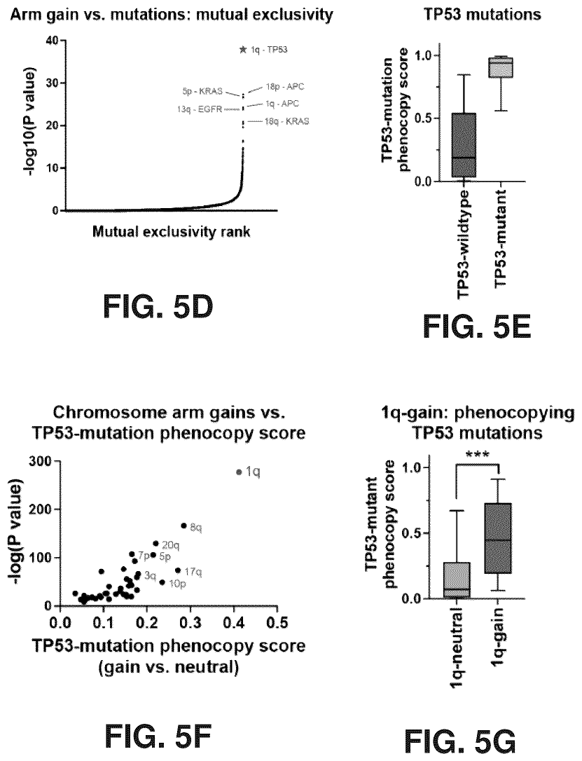

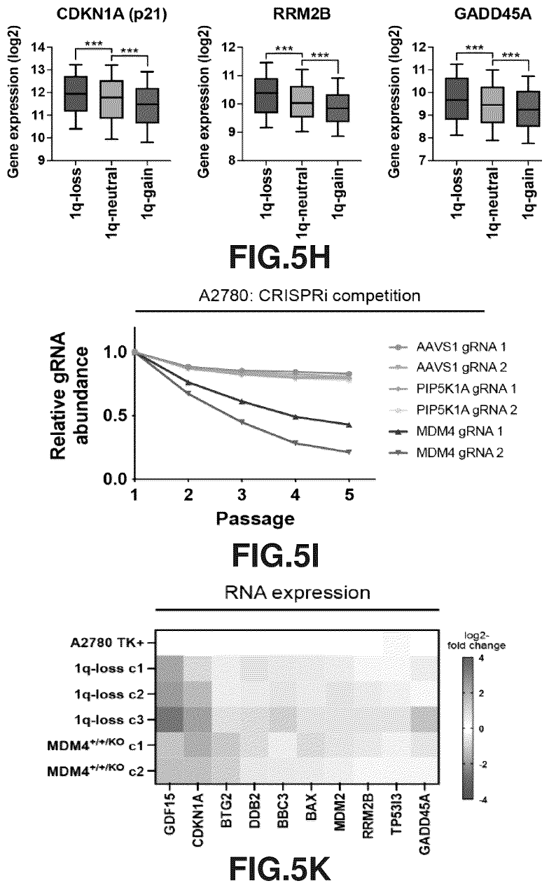

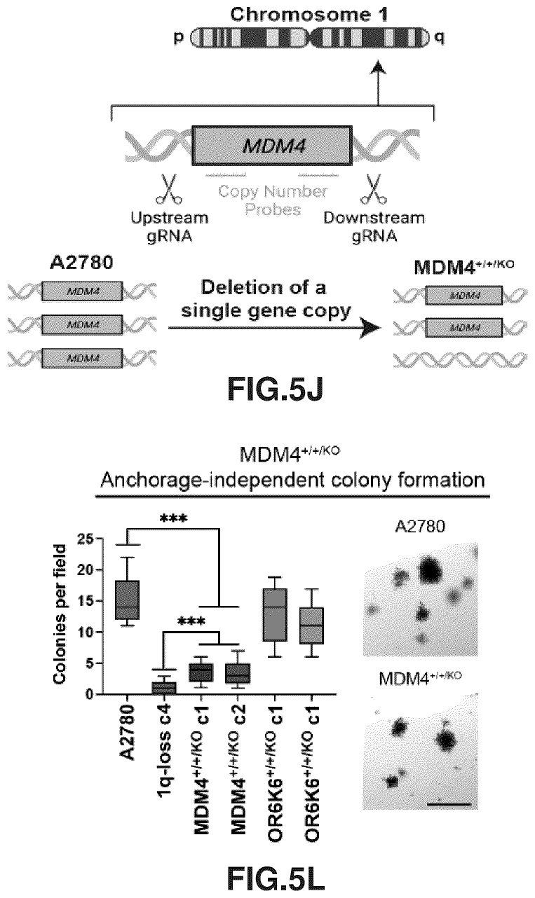

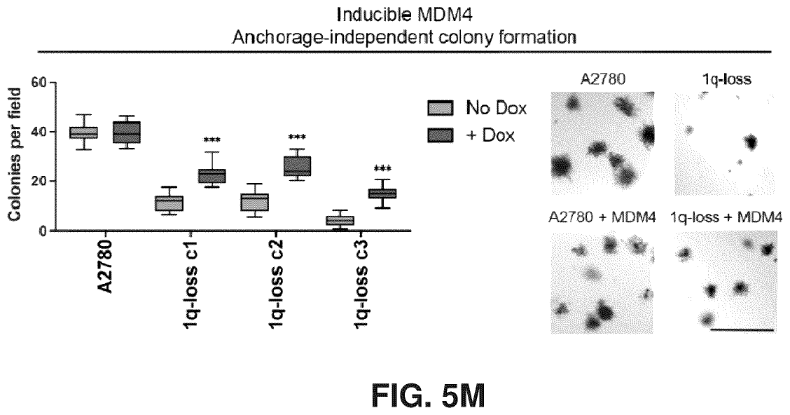

RNA-seq analysis revealed that elimination of the 1q trisomy caused upregulation of tumor suppressor p53 target genes in A2780 and MCF10A, which are both wild-type for TP53 (FIG. 5A-5B). Western blotting confirmed that 1q-disomic clones exhibited increased phosphorylation of p53 at serine-15 and increased expression of the canonical p53 target p21 (FIG. 5C)(Fischer, Oncogene. 36, 3943-3956 (2017)).

These results were not a by-product of CRISPR mutagenesis, as A2780 cells harboring a CRISPR-mediated integration of HSV-TK did not display evidence of p53 activation (FIG. 5B-5C).

Additionally, 1q-disomic A2780 and MCF10A cells exhibited a delay in the G1 phase of the cell cycle and increased senescence-associated beta-galactosidase staining, both of which are associated with p53-mediated tumor suppression (Sugrue, et al., Proc. Natl. Acad. Sci. 94, 9648-9653 (1997)).

Together, these results indicate that the chromosome 1q trisomy inhibits p53 signaling, and elimination of this trisomy antagonizes malignant growth at least in part by triggering p53 activation.

To explore whether p53 inhibition is a common consequence of chromosome 1q gains, the prior analysis of aneuploidy-mutation mutual exclusivity was examined in cancer genomes. Out of 14,383 aneuploidy-gene mutation pairs, the single strongest instance of mutual exclusivity was between 1q gains and TP53 mutations (FIG. 5D).

A classification algorithm capable of predicting cancers that lack p53 function based on their transcriptional profiles was then applied (Fito-Lopez, et al., Prevalence, causes and impact of TP53-loss phenocopying events in human tumors (2022)). Cancers from TCGA with non-synonymous TP53 mutations scored higher with this classifier than cancers with wild-type TP53 (FIG. 5E).

Considering only tumors with wild-type TP53, the association between the p53 status classifier and every possible chromosome arm gain in TCGA was calculated. Across all chromosomes, 1q gains exhibited the strongest correlation with the p53-loss signature (FIG. 5F-5G). Among tumors with wild-type TP53, gains of chromosome 1q were associated with lower expression of the p53 target genes CDKN1A (p21), GADD45A, and RRM2B (FIG. 5H)(Fischer, Oncogene. 36, 3943-3956 (2017)).

Together, these results indicate that gaining chromosome 1q phenocopies the effects of p53 mutations and suppresses p53 activity in human tumors.

The gene(s) on chromosome 1q responsible for inhibiting p53 signaling were investigated. MDM4, a negative regulator of p53 activity, is located on 1q32 (Karni-Schmidt, et al., Annu. Rev. Pathol. Mech. Dis. 11, 617-644 (2016)). MDM4 expression increased with chromosome 1q copy number and higher MDM4 expression correlated with the p53-loss transcriptional signature.

To uncover whether MDM4 is directly responsible for the 1q-aneuploidy addiction observed in A2780, CRISPR-interference (CRISPRi) was used to downregulate MDM4 expression without fully ablating it (Horlbeck, et al., eLife. 5, e19760 (2016)). In A2780 competition assays, downregulating MDM4 impaired cell fitness relative to A2780 cells in which AAVS1 or PIP5K1A, an unrelated gene on chromosome 1q, were downregulated (FIG. 5I)(Girish & Sheltzer, Bio-Protoc. 10, e3682-e3682 (2020)).

Next, a two-guide strategy was used to delete a single copy of MDM4 in an otherwise trisomic background (FIG. 5J).

The subsequent A2780 MDM4+/+/KO clones downregulated MDM4 and upregulated p53 target genes, comparable to the effects observed in cells lacking the entire 1q trisomy (FIG. 5K). The colony formation ability of MDM4+/+/KO clones was then tested, and it was discovered that losing a single copy of MDM4 decreased anchorage-independent growth (FIG. 5L).

Subsequently, the converse experiment was performed: MDM4 cDNA was cloned under the control of a doxycycline-inducible promoter and the construct was transduced into both 1q-trisomic and 1q-disomic cells. Moderate (1.7-fold) overexpression of MDM4 was sufficient to cause an increase in anchorage-independent growth in the 1q-disomic cells, while this same treatment did not affect the 1q-trisomic cells (FIG. 5M).

Finally, to investigate the role of p53 as a mediator of 1q-aneuploidy addiction from an orthogonal approach, CRISPR was used to delete the TP53 gene in A2780 1q-disomic and 1q-trisomic cells. Loss of TP53 rescued the G1 delay and enhanced anchorage-independent growth in 1q-disomic cells. The magnitude of the increase in colony formation was significantly greater in the 1q-disomic cells compared to the 1q-trisomic cells (4-fold vs. 1.5-fold; P<0.0001, t-test).

Together, these results indicate that chromosome 1q gains are a mechanism by which TP53-wildtype cancers can suppress p53 activity, and this suppression occurs due to the increased expression of MDM4.

BCL9 Promotes the Growth of 1q-Aneuploid Cancers Via a p53-Independent Mechanism

The deletion of TP53 and the overexpression of MDM4 in 1q-disomic clones did not fully restore anchorage-independent growth to 1q-trisomic levels (FIG. 5M). It was hypothesized that there were additional dosage-sensitive genes encoded on chromosome 1q that promote the fitness of 1q-aneuploid cancers. To uncover these genes, a panel of 16 1q genes that have previously been associated with tumorigenesis was assembled, including MDM4 as a positive control, and CRISPRi competition assays were conducted to assess the effects of downregulating each gene in 1q-trisomic A2780 cells. gRNAs targeting six genes, including MDM4, exhibited a mean depletion >1.75-fold in cellular competitions. cDNAs for these six genes were cloned into doxycycline-inducible vectors and transduced them into A2780 1q-disomic cells. Overexpression of three of these genes increased anchorage-independent growth: MDM4, the anti-apoptotic gene MCL1, and the Wnt/β-catenin signaling gene BCL9. Two further analyses were then conducted to test whether the effects of the latter two genes were independent of the MDM4/p53 pathway:
first, 1q-disomic cells were co-transduced with vectors to overexpress both MDM4 and BCL9 or MDM4 and MCL1; and
second, the effects of overexpressing BCL9 or MCL1 in TP53-KO 1q-disomic cells were assessed.

MCL1 had no effect on anchorage-independent growth in 1q-disomic cells that lacked TP53, and co-expressing MCL1 and MDM4 in p53-wildtype 1q-disomic cells did not increase clonogenicity beyond the effects of expressing MCL1 alone.

As both MCL1 and TP53 control apoptosis, it was considered that the overexpression of MCL1 and MDM4 are to some extent epistatic with one another (Leu, et al., Nat. Cell Biol. 6, 443-450 (2004)).

In contrast, BCL9 expression enhanced anchorage-independent growth in TP53-KO cells and BCL9-MDM4 co-expression resulted in more colony formation compared to BCL9 alone. BCL9 encodes an adaptor protein that binds to nuclear P-catenin and enhances β-catenin-mediated transcriptional activity (Mani, et al., Cancer Res. 69, 7577-7586 (2009)). Eliminating the trisomy of chromosome 1q reduced the expression of BCL9 as well as AXIN2 and LGR5, which are canonical targets of the Wnt/β-catenin pathway (Nusse, et al., Cell. 169, 985-999 (2017)). GSEA revealed a general decrease in the expression of transcripts associated with the Wnt/β-catenin pathway in 1q-disomic vs. 1q-trisomic cells.

Ectopic overexpression of BCL9 increased the expression of AXIN2 and LGR5. In human cancers, chromosome 1q gains were associated with higher levels of BCL9 expression, and high BCL9 was associated with the upregulation of AXIN2 and LGR5. Mutations in CTNNB1, which encodes P-catenin, were over-represented in cancers with 1q gains. Finally, 1q aneuploidy was associated with higher P-catenin activity in several cancer types, including ovarian cancer, hepatocellular carcinoma, and rectal adenocarcinoma.

These results indicate that chromosome 1q aneuploidy can enhance oncogenic Wnt/β-catenin signaling via the upregulation of BCL9, and increased BCL9 expression promotes cancer cell fitness in a p53-independent manner.

### Example 2: System to Discover Aneuploidy-Selective Cancer Therapies

### Strategy

A CRISPR-based approach that allows genetic elimination of aneuploid chromosomes from cancer cells to discover aneuploidy-selective cancer therapies has been established. Therefore, isogenic “aneuploid” and “aneuploidy-loss” cancer cells can be screened to identify aneuploidy-selective therapies. The platform can serve as a discovery tool for the nomination of both drug candidates, as well as synergistic drug combinations targeting dosage-sensitive genes located on aneuploid chromosomes.

### Methods

A platform for the successful discovery of aneuploidy-specific vulnerabilities was developed: first, several CRISPR-based tools for the targeted manipulation of individual chromosomal arms were developed and, using these, isogenic cell lines that differ in the copy number of a single aneuploid chromosomal arm in a near-diploid background were generated, thereby providing models to study the fitness contributions of a single aneuploid chromosomal arm; and
second, anchorage-independent growth assays were identified as a robust system for the detection of fitness deficits conferred by loss of aneuploid chromosomes (FIG. 2B)

By running various in vitro & in vivo assays in parallel, including proliferation assays, soft agar assays, and xenografts, it was demonstrated that aneuploidy of chromosome 1q, and to a lesser extent that of chromosome 8q, acts as a driver for diverse cancer types, including ovarian, gastric, and colorectal cancer.

Using these assays, it was demonstrated that not all aneuploidies have this effect—for example, aneuploidy of chromosome 7p in melanoma does not make a tangible contribution to the fitness of these cells.

Thirdly, both unbiased screening as well as hypothesis-driven testing approaches were leveraged to identify the dosage-sensitive genes that act as drivers for a particular aneuploidy. Computational pipelines that leverage publicly-available data to verify and nominate dosage-sensitive candidate genes on the aneuploid chromosome are also implemented.

By knocking down candidate genes in the parental, aneuploid setting, and by overexpressing them in the non-aneuploid, engineered lines, several candidates whose change in dosage is partially necessary and sufficient for the fitness benefit of the aneuploid chromosome were identified.

Consequently, a platform for the discovery of genes whose overexpression increases sensitivity to specific anti-cancer compounds (such as drug importer pumps and enzymes that catalyze rate-limiting steps in essential biosynthesis pathways) has been developed and tested to facilitate discovery of aneuploidy-specific vulnerabilities across all cancer types.

Positive-negative selection is a two-step strategy. In the first step, a cassette containing several selectable markers is integrated at a centromere proximal location on the aneuploid chromosome. In the second step, arm loss is induced by creating a dsDNA break in the region between the cassette and the centromere, and negatively selecting for cells that have lost the aneuploid chromosome. The artificial telomere strategy achieves chromosomal arm loss in a single step by capping the induced dsDNA break using an artificial telomere, thereby facilitating chromosomal arm loss.

### Results

### Aneuploidy Addictions Create Collateral Therapeutic Vulnerabilities

The oncogene addiction hypothesis is the conceptual foundation for the use of targeted therapies in cancer (Torti & Trusolino, EMBO Mol. Med. 3, 623-636 (2011)). Correspondingly, whether aneuploidy addictions could also represent a therapeutic vulnerability for certain cancers was investigated.

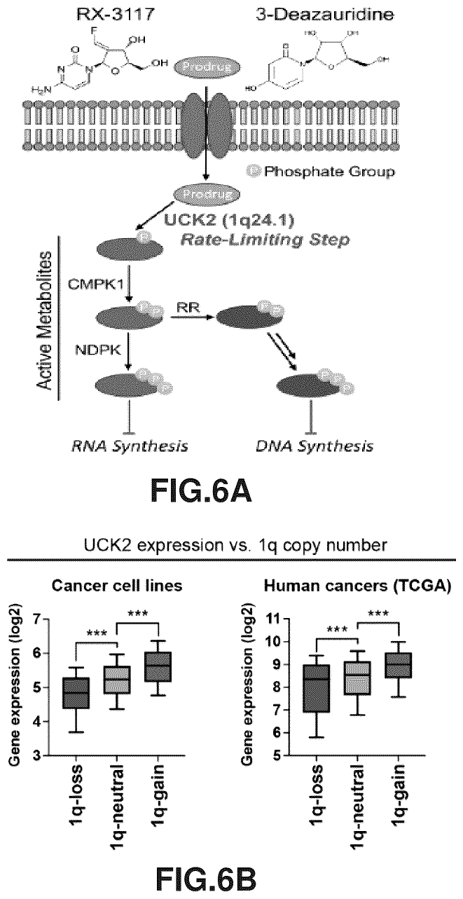

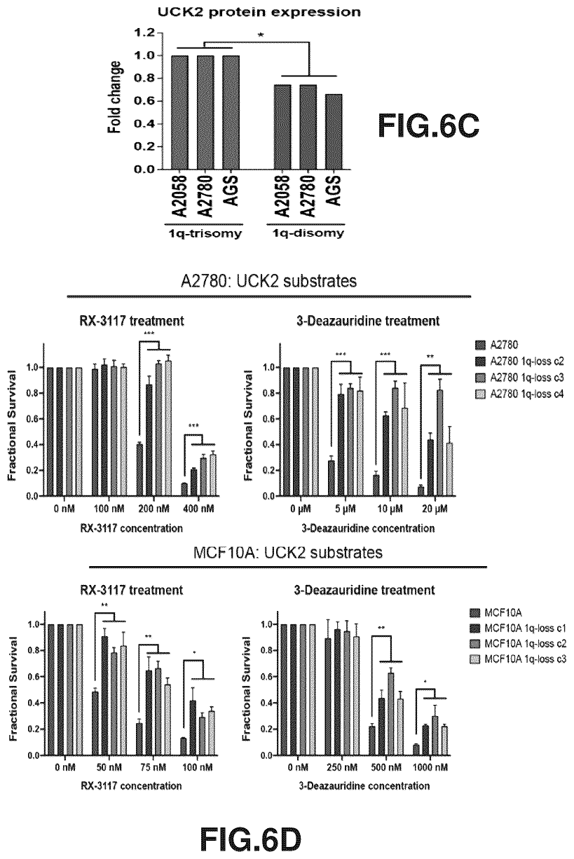

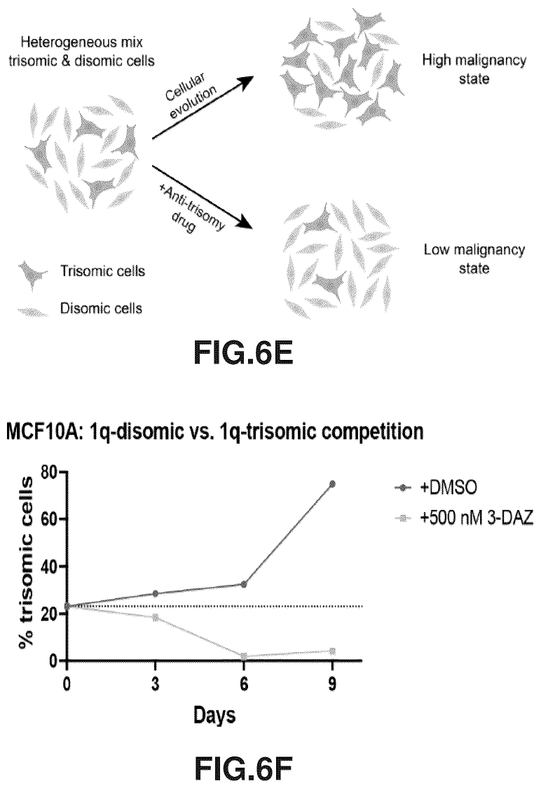

Chromosome 1q harbors the UCK2 gene, which encodes a pyrimidine salvage kinase that controls a rate-limiting step in the activation of certain toxic nucleotide analogs, including RX-3117 and 3-deazauridine (FIG. 6A)(Van Rompay, et al., 59, 1181-1186 (2001), Sarkisjan, et at., PLOS ONE. 11, e0162901 (2016)). UCK2 is overexpressed in human cancers that contain extra copies of chromosome 1q, and elimination of the chromosome 1q trisomy consistently decreased UCK2 protein expression in the engineered cell lines (FIG. 6B-6C). Therefore, whether gaining chromosome 1q could create a collateral sensitivity to UCK2-dependent nucleotide analogs was investigated.

First, as the mechanism of many cancer drugs is poorly understood (Lin, et al., Sci. Transl. Med. 11 (2019), Lin & Sheltzer, Nat. Rev. Genet. 21, 671-682 (2020)),whether the cytotoxicity of RX-3117 and 3-deazauridine requires UCK2 expression was assessed. CRISPR was used to delete UCK2 in the haploid HAP1 cell line, and it was confirmed that UCK2-knockout cells were highly resistant to both compounds.

Next, the effects of RX-3117 and 3-deazauridine were tested in an engineered 1q-trisomic and 1q-disomic cell line. It was found that A2780 and MCF10A cells harboring a trisomy of chromosome 1q were more sensitive to both compounds compared to isogenic cells containing two copies of chromosome 1q (FIG. 6D). This effect was specific for UCK2 substrates, as the 1q-trisomic cells did not exhibit greater sensitivity to UCK2-independent nucleotide poisons and other cancer drugs. Furthermore, deletion of a single copy of UCK2 in 1q-trisomic A2780 cells was sufficient to decrease sensitivity to RX-3117, while ectopic overexpression of UCK2 cDNA in 1q-disomic A2780 cells was sufficient to increase sensitivity to RX-3117. However, a difference in RX-3117 sensitivity between 1q-trisomic and 1q-disomic A2058 and AGS cells was not evident. As A2780 and MCF10A harbor wild-type TP53, while AGS and A2058 exhibit compromised p53 signaling, it was suspected that TP53 status or the expression of other related proteins may also influence the response to UCK2 substrates.

To determine whether 1q copy number changes generated without using ReDACT could also increase sensitivity to UCK2 substrates, near-diploid DLD1 colon cancer cells were transiently treated with an inhibitor of the spindle checkpoint kinase Mps1, and a clone that harbored a trisomy of chromosome 1 was isolated. DLD1 trisomy-1 cells were significantly more sensitive to RX-3117 compared to the parental DLD1 cells (P<0.005; t-test).

Finally, RX-3117 and 3-deazauridine have been screened across the NCI-60 cell line panel, and it was found that higher UCK2 expression correlates with greater sensitivity to both compounds (Luna, et al., Nucleic Acids Res. 49, D1083-D1093 (2021)).

These results indicate that 1q gains induce a collateral sensitivity to certain nucleotide analogs by increasing the expression of UCK2.

Whether the greater sensitivity of 1q-trisomic cells to UCK2 substrates could be used to re-direct cellular evolution away from aneuploidy and towards a disomic state with lower malignant potential was assessed (FIG. 6E). Fluorescently-labeled 1q-trisomic and 1q-disomic MCF10A cells were mixed at a ratio of 20:80 and then co-cultured the two cell populations. After nine days of growth in drug-free medium, the trisomic cells had expanded to include 75% of the culture, but when the same cell populations were grown in the presence of 500 nM 3-deazauridine, the trisomic population decreased to include only 4% of the final culture (FIG. 6F).

How the trisomy to diploidy approach described for 1q could be generalized to identify compounds that exhibit selective toxicity against other aneuploidies was investigated. To nominate drugs that could be used to target chromosome 7p, the PRISM dataset of 4,518 compounds tested against 578 cancer cell lines was assessed (Corsello, et al., Nat. Cancer. 1, 235-248 (2020)).

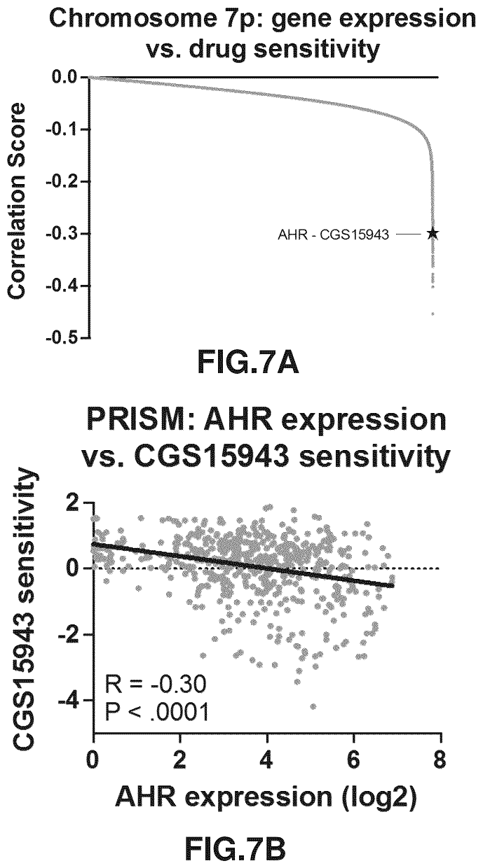

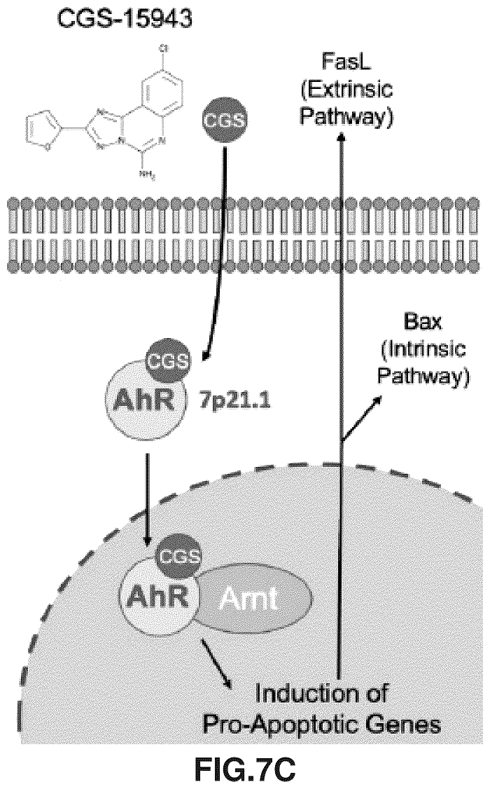

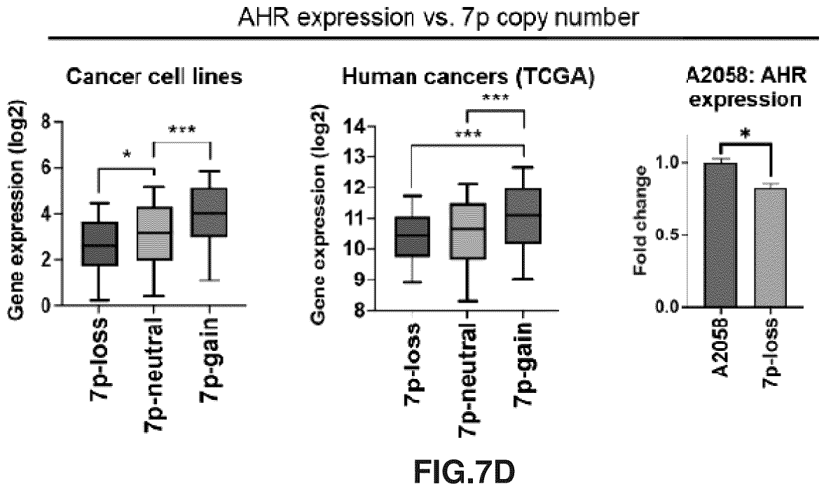

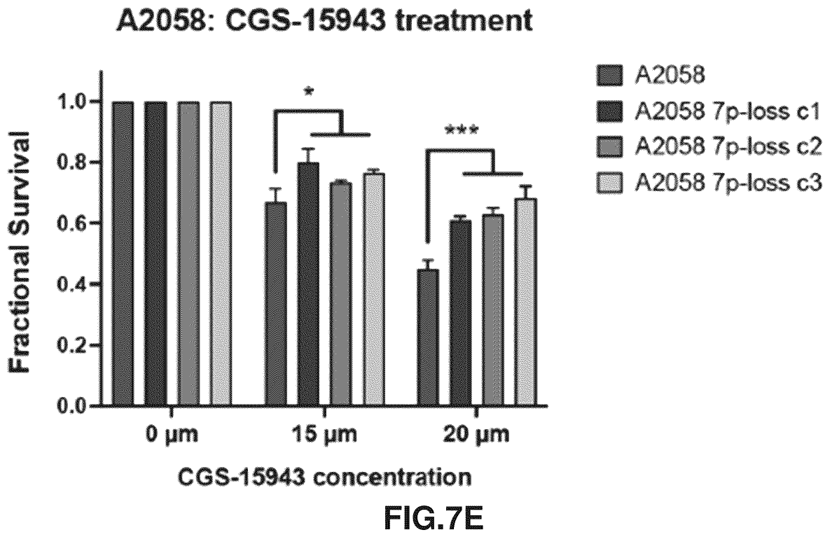

The correlation between the expression of every gene encoded on chromosome 7p and the sensitivity to each drug was determined (FIG. 7A). One of the strongest relationships found was between expression of the gene AHR, which encodes a ligand-activated transcription factor, and sensitivity to the drug CGS-15943 (FIG. 7B).

It has previously been reported that CGS-15943 binds to AHR and causes it to upregulate the expression of pro-apoptotic genes, providing a potential mechanistic explanation for this result (FIG. 7C) (O'Donnell, et al., Apoptosis Int. J. Program. Cell Death. 26, 307-322 (2021)). AHR expression was upregulated in human cancer cell lines and tumors that are trisomic for chromosome 7p, while eliminating the 7p trisomy in the A2058 cell line model decreased the expression of AHR (FIG. 7D).

Consistent with this, A2058 cells with a 7p trisomy were moderately but significantly more sensitive to CGS-15943 compared to 7p-disomic A2058 cells (P<0.0005; t-test;) (FIG. 7E).

Accordingly, it was concluded that trisomy-selective compounds can be used to manipulate cellular evolution to prevent the outgrowth of malignant aneuploid cells in a pre-malignant setting.

It was concluded that, by using a combination of computational and experimental approaches, compounds that exhibit greater activity toward cancer cells with specific aneuploidies can be identified.

### Example 3: 1q Aneuploidy in Cancers is a Biomarker to Predict Therapeutic Efficacy of Chemotherapy

### Strategy

Cancer cell lines harboring extra copies of Chr1q, which encodes the UCK2 gene, exhibit hyper-sensitivity to nucleotide poisons that are activated by UCK2. Whether Chr1q aneuploidy can serve as a biomarker to predict sensitivity to UCK2-activated chemotherapy drugs, including RX-3117 and 3-deazauridine was assessed.

### Methods

A proof-of-principle for this approach in a 1q aneuploidy setting is described in Examples 1 and 2.

Uridine-Cytidine Kinase 2, or UCK2, is a gene located on chromosome 1q that encodes a pyrimidine ribonucleoside kinase which catalyzes the rate-limiting step in the phosphorylation of uridine and cytidine to uridine monophosphate and cytidine monophosphate respectively, prior to their incorporation into nucleic acids.

It has been established that 1q aneuploid cells are more sensitive to drugs targeting UCK2, compared to isogenic, engineered 1q diploid cells using two independent UCK2-targeting drugs, RX-3117 and 3-deazauridine: RX-3117 is a prodrug activated by UCK2, which, upon incorporation into RNA or DNA, induces apoptotic cell death; 3-deazauridine is also phosphorylated by UCK2 and inhibits the biosynthesis of Cytidine-5′-Triphosphate by competitive inhibition of Cytidine Triphosphate synthetase (Mariconi et al., 1986). chromosome 1q aneuploidy has therefore been identified as a biomarker for therapeutic vulnerability to UCK2-targeting chemotherapies.

### Results

The results indicated that 1q Aneuploidy is a Biomarker for Sensitivity to UCK2-Targeting Compounds.

A2780 WT cells are 1q trisomic; C1-C4 are engineered 1q disomic clones. 1q disomic cells are less sensitive to RX-3117 compared to 1q trisomic cells. 1q disomic cells are less sensitive to 3-deazauridine than 1q trisomic cells.

### SUMMARY

Most cancers exhibit aneuploidy, but its functional significance in tumor development has been unknown.

This study describes the development and validation of a set of chromosome engineering tools that enable the elimination of specific aneuploidies from cancer genomes, collectively termed “ReDACT” (Restoring Disomy in Aneuploid cells using CRISPR Targeting).

Using ReDACT, a panel of isogenic cells that have or lack common aneuploidies were created, and it was demonstrated that trisomy of chromosome 1q is required for malignant growth in cancers harboring this alteration. Mechanistically, gaining chromosome 1q increases the expression of MDM4 and suppresses p53 signaling, and it was shown that TP53 mutations are mutually-exclusive with 1q aneuploidy in human cancers. Thus, tumor cells can be dependent on specific aneuploidies, raising the possibility that these “aneuploidy addictions” could be targeted as a therapeutic strategy.

In this work, endogenous aneuploidies were eliminated from established cancer cell lines, and it was demonstrated that the removal of trisomic chromosomes compromises cancer-like growth. These phenotypes are due specifically to the loss of the aneuploid chromosome and are not a by-product of CRISPR selection or the elimination of point mutations encoded on the targeted chromosome (Girish, et al., Supplemental Material).

Due to the similarity between the observations and the previously-described oncogene addiction phenomenon (Weinstein, Science. 297, 63-64 (2002)), it is likely that certain cancers may also be “addicted” to the aneuploidy found in their genomes and that, during tumor evolution, certain aneuploidies can provide context-specific benefits that enhance tumorigenesis. For instance, the data showed that chromosome 1q gains are an early event during cancer development, and it was demonstrated that MDM4 and BCL9 are dosage-sensitive genes on 1q that enhance malignant growth. In cells that already harbor TP53 mutations or in cancer types that are not driven by WNT signaling, the beneficial effects of gaining chromosome 1q may be outweighed by the detrimental effects of overexpressing hundreds of other 1q genes.

MDM4 and many other genes have tumor-promoting properties when highly overexpressed (Santarius, et al., Nat. Rev. Cancer. 10, 59-64 (2010), Hüllein, et al., Cancer Res. 79, 3125-3138 (2019)). For instance, MDM4 is focally amplified in ˜1% of cancers in TCGA, and strong overexpression of MDM4 via retrovirus immortalizes primary cells and renders them sensitive to Ras-mediated transformation (Danovi, et al., Mol. Cell. Biol. 24, 5835-5843 (2004)). The data demonstrated that a single extra copy of MDM4 is sufficient to suppress the expression of p53 target genes and promote oncogenic growth. The results are consistent with the notion that low-level overexpression of MDM4 can enhance fitness in hematopoietic cell competitions (Sebert, et al., Cell Stem Cell. 30, 153-170.e9 (2023)).

The overlap between single-copy dosage-sensitive genes like MDM4 and genes found to have tumor-promoting properties when highly overexpressed is at present unknown. Furthermore, this work established that MDM4 overexpression alone is insufficient to fully recapitulate the oncogenic effects of 1q aneuploidy, and that BCL9 is a second dosage-sensitive 1q gene that enhances fitness in a TP53-independent manner. As arm-length aneuploidy is more common in tumor genomes than focal gene amplifications (Zack, et al., Nat. Genet. 45, 1134-1140 (2013)), it is likely that recurrently-gained chromosomes harbor multiple independent loci like MDM4 and BCL9 that cooperate to drive tumor development.

Finally, these results raise the exciting possibility that “aneuploidy addictions” may represent a therapeutic vulnerability in cancer. Previous attempts to target aneuploidy have focused on phenotypes that are shared across highly-aneuploid cells, such as alterations in spindle geometry (Quinton, et al., Nature. 590, 492-497 (2021), Cohen-Sharir, et al., Nature. 590, 486-491 (2021)). Here, an approach to take advantage of the genes that are encoded on an aneuploid chromosome was developed, thereby allowing chromosome-specific targeting. In particular, experiments were designed to test if overexpression of specific genes—for instance, drug-importer pumps or enzymes required for a prodrug's activation—could sensitize cancers to compounds that are otherwise better-tolerated in euploid tissue. It was demonstrated that gaining chromosome 1q creates a collateral vulnerability to the nucleotide analogs RX-3117 and 3-deazauridine due to the overexpression of the kinase UCK2. Notably, RX-3117 has been tested in phase 2A clinical trials, but without the use of any genomic biomarkers for patient selection (Salgia, et al., J. Clin. Oncol. 37, 455-455 (2019)). High UCK2 expression has been proposed as a potential sensitivity biomarker for RX-3117, and it was speculated that patients whose tumors harbor gains of chromosome 1q may exhibit strong responses due to the constitutive overexpression of UCK2 (El Hassouni, et al., Anticancer Res. 39, 3609-3614 (2019)). More broadly, compounds whose anti-cancer function is enhanced by genes encoded on aneuploid chromosomes could be used to direct cellular evolution away from certain aneuploidies and toward the lower-malignancy diploid state.

### REFERENCES

1. Taylor A. M. et al. Genomic and Functional Approaches to Understanding Cancer Aneuploidy. Cancer Cell 33, 676-689.e3 (2018).
2. Gordon D. J., Resio B. & Pellman D. Causes and consequences of aneuploidy in cancer. Nat. Rev. Genet. 13, 189-203 (2012).
3. Boveri T. Concerning the Origin of Malignant Tumours by Theodor Boveri. Translated and annotated by Henry Harris. J. Cell Sci. 121, 1-84 (2008).
4. Hardy P. A. & Zacharias H. Reappraisal of the Hansemann-Boveri hypothesis on the origin of tumors. Cell Biol. Int. 29, 983-992 (2005).
5. Vasudevan A. et al. Aneuploidy as a promoter and suppressor of malignant growth. Nat. Rev. Cancer 21, 89-103 (2021).
6. Ben-David U. & Amon A. Context is everything: aneuploidy in cancer. Nat. Rev. Genet. 21, 44-62 (2020).
7. Sheltzer J. M. & Amon A. The aneuploidy paradox: costs and benefits of an incorrect karyotype. Trends Genet. TIG 27, 446-453 (2011).
8. Weaver B. A. A. & Cleveland D. W. Does aneuploidy cause cancer?Curr. Opin. Cell Biol. 18, 658-667 (2006).
9. Davoli T. et al. Cumulative Haploinsufficiency and Triplosensitivity Drive Aneuploidy Patterns to Shape the Cancer Genome. Cell 155, 948-962 (2013).
10. Sack L. M. et al. Profound Tissue Specificity in Proliferation Control Underlies Cancer Drivers and Aneuploidy Patterns. Cell 173, 499-514.e23 (2018).
11. Zimonjic D., Brooks M. W., Popescu N., Weinberg R. A. & Hahn W. C. Derivation of Human Tumor Cells in Vitro without Widespread Genomic Instability. Cancer Res. 61, 8838-8844 (2001).
12. Satgé D. et al. A tumor profile in Down syndrome. Am. J. Med. Genet. 78, 207-216 (1998).
13. Hasle H., Friedman J. M., Olsen J. H. & Rasmussen S. A. Low risk of solid tumors in persons with Down syndrome. Genet. Med. 18, 1151-1157 (2016).
14. Bister K. Discovery of oncogenes: The advent of molecular cancer research. Proc. Natl. Acad. Sci. U.S.A 112, 15259-15260 (2015).
15. O'Loughlin T. A. & Gilbert L. A. Functional Genomics for Cancer Research: Applications In Vivo and In Vitro. Annu. Rev. Cancer Biol. 3, 345-363 (2019).
16. Bunz F. et al. Requirement for p53 and p21 to sustain G2 arrest after DNA damage. Science 282, 1497-1501 (1998).
17. Shirasawa S., Furuse M., Yokoyama N. & Sasazuki T. Altered growth of human colon cancer cell lines disrupted at activated Ki-ras. Science 260, 85-88 (1993).
18. Weinstein I. B. Addiction to Oncogenes—the Achilles Heal of Cancer. Science 297, 63-64 (2002).
19. Waters A. M. & Der C. J. KRAS: The Critical Driver and Therapeutic Target for Pancreatic Cancer. Cold Spring Harb. Perspect. Med. 8, (2018).
20. Sharma S. V. & Settleman J. Oncogene addiction: setting the stage for molecularly targeted cancer therapy. Genes Dev. 21, 3214-3231 (2007).
21. Ono M. et al. Sensitivity to gefitinib (Iressa, ZD1839) in non-small cell lung cancer cell lines correlates with dependence on the epidermal growth factor (EGF) receptor/extracellular signal-regulated kinase 1/2 and EGF receptor/Akt pathway for proliferation. Mol. Cancer Ther. 3, 465-472 (2004).
22. Chapman P. B. et al. Improved Survival with Vemurafenib in Melanoma with BRAF V600E Mutation. N. Engl. J. Med. 364, 2507-2516 (2011).
23. Knouse K. A., Davoli T., Elledge S. J. & Amon A. Aneuploidy in Cancer: Seq-ing Answers to Old Questions. Annu. Rev. Cancer Biol. 1, 335-354 (2017).
24. Beroukhim R. et al. The landscape of somatic copy-number alteration across human cancers. Nature 463, 899-905 (2010).
25. Zack T. I. et al. Pan-cancer patterns of somatic copy number alteration. Nat. Genet. 45, 1134-1140 (2013).
26. McGranahan N. et al. Clonal status of actionable driver events and the timing of mutational processes in cancer evolution. Sci. Transl. Med. 7, 283ra54-283ra54 (2015).
27. Wang Z. et al. Evolving copy number gains promote tumor expansion and bolster mutational diversification. 2022.06.14.495959 Preprint at 10.1101/2022.06.14.495959 (2022).
28. Yates L. R. et al. Genomic Evolution of Breast Cancer Metastasis and Relapse. Cancer Cell 32, 169-184.e7 (2017).
29. Hayward N. K. et al. Whole-genome landscapes of major melanoma subtypes. Nature 545, 175-180 (2017).
30. Paterson C., Clevers H. & Bozic I. Mathematical model of colorectal cancer initiation. Proc. Natl. Acad. Sci. 117, 20681-20688 (2020).
31. Cisowski J. & Bergo M. O. What makes oncogenes mutually exclusive?Small GTPases 8, 187-192 (2017).
32. El Tekle G. et al. Co-occurrence and mutual exclusivity: what cross-cancer mutation patterns can tell us. Trends Cancer 7, 823-836 (2021).
33. Gorden A. et al. Analysis of BRAF and N-RAS Mutations in Metastatic Melanoma Tissues. Cancer Res. 63, 3955-3957 (2003).
34. Nguyen B. et al. Genomic characterization of metastatic patterns from prospective clinical sequencing of 25,000 patients. Cell 185, 563-575.ell (2022).
35. Stopsack K. H. et al. Aneuploidy drives lethal progression in prostate cancer. Proc. Natl. Acad. Sci. 116, 11390-11395 (2019).
36. Hieronymus H. et al. Tumor copy number alteration burden is a pan-cancer prognostic factor associated with recurrence and death. eLife 7, e37294 (2018).
37. Lukow D. A. et al. Chromosomal instability accelerates the evolution of resistance to anti-cancer therapies. Dev. Cell 56, 2427-2439.e4 (2021).
38. Luo J., Solimini N. L. & Elledge S. J. Principles of Cancer Therapy: Oncogene and Non-oncogene Addiction. Cell 136, 823-837 (2009).
39. Fillat C., Carrio M., Cascante A. & Sangro B. Suicide Gene Therapy Mediated by the Herpes Simplex Virus Thymidine Kinase Gene/Ganciclovir System: Fifteen Years of Application. Curr. Gene Ther. 3, 13-26 (2003).
40. Uno N. et al. CRISPR/Cas9-induced transgene insertion and telomere-associated truncation of a single human chromosome for chromosome engineering in CHO and A9 cells. Sci. Rep. 7, 12739 (2017).
41. Zuccaro M. V. et al. Allele-Specific Chromosome Removal after Cas9 Cleavage in Human Embryos. Cell 183, 1650-1664.e15 (2020).
42. Papathanasiou S. et al. Whole chromosome loss and genomic instability in mouse embryos after CRISPR-Cas9 genome editing. Nat. Commun. 12, 5855 (2021).
43. Wang Z. et al. SMASH, a fragmentation and sequencing method for genomic copy number analysis. Genome Res. 26, 844-851 (2016).
44. Schukken K. M. & Sheltzer J. Extensive protein dosage compensation in aneuploid human cancers. Genome Res. gr.276378.121 (2022) doi: 10.1101/gr.276378.121.
45. McShane E. et al. Kinetic Analysis of Protein Stability Reveals Age-Dependent Degradation. Cell 167, 803-815.e21 (2016).
46. Shin S. I., Freedman V. H., Risser R. & Pollack R. Tumorigenicity of virus-transformed cells in nude mice is correlated specifically with anchorage independent growth in vitro. Proc. Natl. Acad. Sci. 72, 4435-4439 (1975).
47. Hamburger A. W. & Salmon S. E. Primary bioassay of human tumor stem cells. Science 197, 461-463 (1977).
48. Chin L. et al. Essential role for oncogenic Ras in tumour maintenance. Nature 400, 468-472 (1999).
49. Soule H. D. et al. Isolation and characterization of a spontaneously immortalized human breast epithelial cell line, MCF-10. Cancer Res. 50, 6075-6086 (1990).
50. Irion S. et al. Identification and targeting of the ROSA26 locus in human embryonic stem cells. Nat. Biotechnol. 25, 1477-1482 (2007).
51. Torti D. & Trusolino L. Oncogene addiction as a foundational rationale for targeted anti-cancer therapy: promises and perils. EMBO Mol. Med. 3, 623-636 (2011).
52. Westover D., Zugazagoitia J., Cho B. C., Lovly C. M. & Paz-Ares L. Mechanisms of acquired resistance to first- and second-generation EGFR tyrosine kinase inhibitors. Ann. Oncol. 29, i10-i19 (2018).
53. Burgess M. R. et al. KRAS Allelic Imbalance Enhances Fitness and Modulates MAP Kinase Dependence in Cancer. Cell 168, 817-829.el5 (2017).
54. Mueller S. et al. Evolutionary routes and KRAS dosage define pancreatic cancer phenotypes. Nature 554, 62-68 (2018).
55. Fischer M. Census and evaluation of p53 target genes. Oncogene 36, 3943-3956 (2017).
56. Sugrue M. M., Shin D. Y., Lee S. W. & Aaronson S. A. Wild-type p53 triggers a rapid senescence program in human tumor cells lacking functional p53. Proc. Natl. Acad. Sci. 94, 9648-9653 (1997).
57. Fito-Lopez B., Salvadores M., Alvarez M.-M. & Supek F. Prevalence, causes and impact of TP53-loss phenocopying events in human tumors. 2022.11.01.514743 Preprint at 10.1101/2022.11.01.514743 (2022).
58. Karni-Schmidt O., Lokshin M. & Prives C. The Roles of MDM2 and MDMX in Cancer. Annu. Rev. Pathol. Mech. Dis. 11, 617-644 (2016).
59. Gilbert L. A. et al. Genome-Scale CRISPR-Mediated Control of Gene Repression and Activation. Cell 159, 647-661 (2014).
60. Horlbeck M. A. et al. Compact and highly active next-generation libraries for CRISPR-mediated gene repression and activation. eLife 5, e19760 (2016).
61. Girish V. & Sheltzer J. M. A CRISPR Competition Assay to Identify Cancer Genetic Dependencies. Bio-Protoc. 10, e3682-e3682 (2020).
62. Fu Y. et al. The Metabolic and Non-Metabolic Roles of UCK2 in Tumor Progression. Front. Oncol. 12, 904887 (2022).
63. Van Rompay A. R., Norda A., Lindén K., Johansson M. & Karlsson A. Phosphorylation of uridine and cytidine nucleoside analogs by two human uridine-cytidine kinases. Mol. Pharmacol. 59, 1181-1186 (2001).
64. van Kuilenburg A. B. P. & Meinsma R. The pivotal role of uridine-cytidine kinases in pyrimidine metabolism and activation of cytotoxic nucleoside analogues in neuroblastoma. Biochim. Biophys. Acta BBA—Mol. Basis Dis. 1862, 1504-1512 (2016).
65. Sarkisjan D. et al. The Cytidine Analog Fluorocyclopentenylcytosine (RX-3117) Is Activated by Uridine-Cytidine Kinase 2. PLOS ONE 11, e0162901 (2016).
66. Peters G. J. Novel Developments in the Use of Antimetabolites. Nucleosides Nucleotides Nucleic Acids 33, 358-374 (2014).
67. Lin A. et al. Off-target toxicity is a common mechanism of action of cancer drugs undergoing clinical trials. Sci. Transl. Med. 11, (2019).
68. Lin A. & Sheltzer J. M. Discovering and validating cancer genetic dependencies: approaches and pitfalls. Nat. Rev. Genet. 21, 671-682 (2020).
69. Luna A. et al. CellMiner Cross-Database (CellMinerCDB) version 1.2: Exploration of patient-derived cancer cell line pharmacogenomics. Nucleic Acids Res. 49, D1083-D1093 (2021).
70. Michel L. S. et al. MAD2 haplo-insufficiency causes premature anaphase and chromosome instability in mammalian cells. Nature 409, 355-359 (2001).
71. Schvartzman J.-M., Sotillo R. & Benezra R. Mitotic chromosomal instability and cancer: mouse modelling of the human disease. Nat Rev Cancer 10, 102-115 (2010).
72. Simon J. E., Bakker B. & Foijer F. CINcere Modelling: What Have Mouse Models for Chromosome Instability Taught Us? in Chromosomal Instability in Cancer Cells (eds. Ghadimi B. M. & Ried T.) 39-60 (Springer International Publishing, 2015). doi: 10.1007/978-3-319-20291-4_2.
73. Weaver B. A. A., Silk A. D., Montagna C., Verdier-Pinard P. & Cleveland D. W. Aneuploidy Acts Both Oncogenically and as a Tumor Suppressor. Cancer Cell 11, 25-36 (2007).
74. Silk A. D. et al. Chromosome missegregation rate predicts whether aneuploidy will promote or suppress tumors. Proc. Natl. Acad. Sci. 110, E4134-E4141 (2013).
75. Baker D. J., Jin F., Jeganathan K. B. & van Deursen J. M. Whole Chromosome Instability Caused by BubI Insufficiency Drives Tumorigenesis through Tumor Suppressor Gene Loss of Heterozygosity. Cancer Cell 16, 475-486 (2009).
76. Schukken K. M. & Foijer F. CIN and Aneuploidy: Different Concepts, Different Consequences. BioEssays 40, 1700147 (2018).
77. Bakhoum S. F. & Cantley L. C. The Multifaceted Role of Chromosomal Instability in Cancer and Its Microenvironment. Cell 174, 1347-1360 (2018).
78. Bakhoum S. F. et al. Chromosomal instability drives metastasis through a cytosolic DNA response. Nature 553, 467-472 (2018).
79. Campbell M. S., Chan G. K. & Yen T. J. Mitotic checkpoint proteins HsMAD1 and HsMAD2 are associated with nuclear pore complexes in interphase. J. Cell Sci. 114, 953-963 (2001).
80. Choi E., Zhang X., Xing C. & Yu H. Mitotic Checkpoint Regulators Control Insulin Signaling and Metabolic Homeostasis. Cell 166, 567-581 (2016).
81. Singh N. & Bhalla N. Moonlighting Proteins. Annu. Rev. Genet. 54, 265-285 (2020).
82. Stingele S. et al. Global analysis of genome, transcriptome and proteome reveals the response to aneuploidy in human cells. Mol. Syst. Biol. 8, (2012).
83. Doherty A. M. O. & Fisher E. M. C. Microcell-mediated chromosome transfer (MMCT): small cells with huge potential. Mamm. Genome 14, 583-592 (2003).
84. Passerini V. et al. The presence of extra chromosomes leads to genomic instability. Nat. Commun. 7, ncomms10754 (2016).
85. Sheltzer J. M. et al. Single-chromosome Gains Commonly Function as Tumor Suppressors. Cancer Cell 31, 240-255 (2017).
86. Santaguida S., Vasile E., White E. & Amon A. Aneuploidy-induced cellular stresses limit autophagic degradation. Genes Dev. 29, 2010-2021 (2015).
87. Donnelly N., Passerini V., Dürrbaum M., Stingele S. & Storchovi Z. HSF1 deficiency and impaired HSP90-dependent protein folding are hallmarks of aneuploid human cells. EMBO J. 33, 2374-2387 (2014).
88. Donnelly N. & Storchová Z. Aneuploidy and proteotoxic stress in cancer. Mol. Cell. Oncol. 2, e976491 (2015).
89. Ohashi A. et al. Aneuploidy generates proteotoxic stress and DNA damage concurrently with p53-mediated post-mitotic apoptosis in SAC-impaired cells. Nat. Commun. 6, 7668 (2015).
90. Santarius T., Shipley J., Brewer D., Stratton M. R. & Cooper C. S. A census of amplified and overexpressed human cancer genes. Nat. Rev. Cancer 10, 59-64 (2010).
91. Danovi D. et al. Amplification of Mdmx (or Mdm4) Directly Contributes to Tumor Formation by Inhibiting p53 Tumor Suppressor Activity. Mol. Cell. Biol. 24, 5835-5843 (2004).
92. Quinton R. J. et al. Whole-genome doubling confers unique genetic vulnerabilities on tumour cells. Nature 590, 492-497 (2021).
93. Cohen-Sharir Y. et al. Aneuploidy renders cancer cells vulnerable to mitotic checkpoint inhibition. Nature 590, 486-491 (2021).
94. Salgia N. et al. Activity of RX-3117, an oral antimetabolite nucleoside, in subjects with advanced urothelial cancer: Preliminary results of a phase IIa study. J. Clin. Oncol. 37, 455-455 (2019).
95. Chang K., Marran K., Valentine A. & Hannon G. J. Packaging shRNA retroviruses. Cold Spring Harb. Protoc. 2013, 734-737 (2013).
96. Giuliano C. J., Lin A., Girish V. & Sheltzer J. M. Generating Single Cell-Derived Knockout Clones in Mammalian Cells with CRISPR/Cas9. Curr. Protoc. Mol. Biol. 128, e100 (2019).
97. Sanson K. R. et al. Optimized libraries for CRISPR-Cas9 genetic screens with multiple modalities. Nat. Commun. 9, (2018).
98. Di Tommaso P. et al. Nextflow enables reproducible computational workflows. Nat. Biotechnol. 35, 316-319 (2017).
99. Andrews P. A. et al. MUMdex: MUM-based structural variation detection. bioRxiv 078261 (2016) doi: 10.1101/078261.
100. Wickham H. et al. Welcome to the Tidyverse. J. Open Source Softw. 4, 1686 (2019).
101. Mayo P. et al. CNV Analysis Using TaqMan Copy Number Assays. Curr. Protoc. Hum. Genet. 67, 2.13.1-2.13.10 (2010).
102. Vasudevan A. et al. Single-Chromosomal Gains Can Function as Metastasis Suppressors and Promoters in Colon Cancer. Dev. Cell 52, 413-428.e6 (2020).
103. Liu Y. et al. Multi-omic measurements of heterogeneity in HeLa cells across laboratories. Nat. Biotechnol. 37, 314-322 (2019).
104. Collins B. C. et al. Multi-laboratory assessment of reproducibility, qualitative and quantitative performance of SWATH-mass spectrometry. Nat. Commun. 8, 291 (2017).
105. Mehnert M., Li W., Wu C., Salovska B. & Liu Y. Combining Rapid Data Independent Acquisition and CRISPR Gene Deletion for Studying Potential Protein Functions: A Case of HMGN1. Proteomics 19, e1800438 (2019).
106. Bruderer R. et al. Optimization of Experimental Parameters in Data-Independent Mass Spectrometry Significantly Increases Depth and Reproducibility of Results. Mol. Cell. Proteomics MCP 16, 2296-2309 (2017).
107. Bruderer R. et al. Analysis of 1508 Plasma Samples by Capillary-Flow Data-Independent Acquisition Profiles Proteomics of Weight Loss and Maintenance. Mol. Cell. Proteomics MCP 18, 1242-1254 (2019).
108. Bruderer R. et al. Extending the limits of quantitative proteome profiling with data-independent acquisition and application to acetaminophen-treated three-dimensional liver microtissues. Mol. Cell. Proteomics MCP 14, 1400-1410 (2015).
109. Tsou C.-C. et al. DIA-Umpire: comprehensive computational framework for data-independent acquisition proteomics. Nat. Methods 12, 258-264, 7 p following 264 (2015).
110. Smyth G. K. limma: Linear Models for Microarray Data. in Bioinformatics and Computational Biology Solutions Using R and Bioconductor (eds. Gentleman R., Carey V. J., Huber W., Irizarry R. A. & Dudoit S. 397-420 (Springer, 2005). doi: 10.1007/0-387-29362-0_23.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nucleic acid sequence is disclosed and discussed and a number of modifications that can be made to a number of molecules including the nucleic acid sequence are discussed, each and every combination and permutation of the nucleic acid sequence and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a “an”, and “the” include plural reference unless the context clearly dictates otherwise. Thus, for example, reference to “a nucleic acid sequence” includes a plurality of such nucleic acids, reference to “the nucleic acids” is a reference to one or more nucleic acid and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicate an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated, and considered disclosed is the range from the one particular value and/or to the other particular value unless the context specifically indicates otherwise. Similarly, when values are expressed as approximations, by use of the antecedent “about,” it will be understood that the value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. All of the individual values and sub-ranges of values contained within an explicitly disclosed range are also specifically contemplated and should be considered disclosed unless the context specifically indicates otherwise. Finally, all ranges refer both to the recited range as a range and as a collection of individual numbers from and including the first endpoint to and including the second endpoint. In the latter case, any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents form part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different gene targets does not indicate that the listed gene targets are obvious one to the other, nor is it an admission of equivalence or obviousness.

Every component disclosed herein is intended to be and should be considered to be specifically disclosed herein. Further, every subgroup that can be identified within this disclosure is intended to be and should be considered to be specifically disclosed herein. As a result, it is specifically contemplated that any component, or subgroup of components can be either specifically included for or excluded from use or included in or excluded from a list of components.

Those skilled in the art will recognize, or be able to ascertain using no more than routine experimentation, many equivalents to the specific embodiments of the method and compositions described herein. Such equivalents are intended to be encompassed by the following claims.

We claim:

1. A method for creating a viable isogenic cell or tissue or organoid having an ablated aneuploid chromosome or a fragment thereof from a precursor aneuploid cell or tissue or organoid, comprising:
(i) introducing into a precursor cell or tissue or organoid a gene editing composition configured to cleave a target chromosome or target chromosome arm and optionally a nucleic acid configured to integrate into the target chromosome or target chromosome arm to form an isogenic cell or tissue comprising a cleaved target chromosome or cleaved target chromosome arm; or
and
(ii) optionally isolating the isogenic cell or tissue or organoid,
optionally wherein the cleaved aneuploid chromosome arm is not present in the isogenic the cell or tissue or organoid.

2. The method of claim 1, wherein the precursor cell or tissue or organoid is an aneuploid cell or tissue or organoid.

3. The method of claim 1, wherein the target chromosome is an aneuploid chromosome or a fragment thereof.

4. The method of claim 1, wherein the viable isogenic cell or tissue or organoid is a viable isogenic diploid-like cell or tissue or organoid.

5. The method of claim 1, wherein the viable isogenic cell or tissue or organoid is disomic for the aneuploid arm.

6. The method of claim 1, wherein the gene editing composition is selected from the group consisting of Clustered Regularly Interspaced Short Palindromic Repeats/CRISPR associated protein (CRISPR/Cas), zinc finger nucleases (ZFN), and transcription activator-like effector nucleases (TALEN), optionally wherein the gene editing composition comprises a CRISPR/Cas composition optionally comprising a guide RNA (gRNA) configured to cleave nucleotides proximal to a centromere in the target chromosome arm.

7. The method of claim 6, wherein the gene editing composition comprises a CRISPR/Cas composition,
wherein the nucleic acid configured to integrate into the target chromosome arm comprises a reporter gene,
wherein the CRISPR/Cas composition is configured to cleave the target chromosome arm between the integrated reporter gene and a centromere of the target chromosome arm; and
wherein isolating the isogenic cell or tissue comprises selecting a cell or tissue lacking the reporter gene.

8. The method of claim 6, wherein the gene editing composition comprises a CRISPR/Cas composition,
wherein the nucleic acid configured to integrate into the target chromosome arm comprises a telomere repeat sequence,
wherein the CRISPR/Cas composition is configured to cleave nucleotides proximal to a centromere in the target chromosome arm and integrate the telomere repeat sequence to provide a de novo telomere; and
wherein isolating the isogenic cells or tissue comprises selecting cells or tissue comprising the de novo telomere.

9. The method of claim 1, wherein the cell or tissue or organoid is a human cell or tissue, and
wherein the target chromosome is selected from the group consisting of chromosome 1, 2, 3, 4, 5, 6, 7, 8, 9, 10, 11, 12, 13, 14, 15, 16, 17, 18, 19, 20, 21, 22, X, or Y;
or
wherein the target chromosome arm is selected from the group consisting of arm 1q, 1p, 2q, 2p, 3q, 3p, 4q, 4p, 5q, 5p, 6q, 6p, 7p, 7q, 8p, 8q, 9p, 9q, 10p, 10q, lip, 11q, 12p, 12q, 13p, 13q, 14q, 14p, 15q, 15p, 16p, 16q, 17p, 17q, 18p, 18q, 19q, 19q, 20q, 20p, or 21q, 21p, 22q, 22p, Xp, Xq, Yp, or Yq.

10. The method of claim 1, wherein the target chromosome or target chromosome arm expresses one or more genes selected from the group consisting of CYP2J2, MFSD2A, UCK1, UCK2, SLC16A1, UGT1A10, DCK, SLC26A2, SLC29A1, ACTB, ABCB1, SLC39A4, ADK, FADS2, SLC35F2, PDE3A, ABCC1, SULT1A1, NQO1, SLC47A1, SLFN11, SLFN12, DCAF15, SLC6A16, SLC19A1, and AHR.

11. The method of claim 1, wherein the precursor cell or tissue is a cancer cell or cancer tissue or cancer organoid,
optionally wherein the cancer cell or tissue is an aneuploid cancer cell or an aneuploid cancer tissue,
optionally wherein the aneuploid cancer cell or an aneuploid cancer tissue is derived from a subject with cancer.

12. The method of claim 11, further comprising prior to step (i), obtaining the aneuploid cancer cell or tissue from the subject.

13. The method of claim 11, wherein the cancer is selected from the group consisting of anal cancer, bladder cancer, breast cancer, cervical cancer, colorectal cancer, endometrial cancer, esophagogastric cancer, gastrointestinal neuro-endo-carcinoma, gastrointestinal stromal tumor, head and neck cancer, hepatobiliary cancer, melanoma, mesothelioma, non-small cell lung cancer, ovarian cancer, prostate cancer, pancreatic cancer, renal carcinoma, salivary gland cancer, non-melanoma skin cancer, small bowel cancer, small cell lung cancer, soft-tissue sarcoma, thyroid cancer, uterine cancer, acute myeloid leukemia, and adrenocortical carcinoma.

14. The method of claim 1, wherein the precursor cell or tissue is or is part of a tissue graft, a prosthesis or an organoid.

15. An isogenic cell or tissue or organoid formed from a precursor cell or tissue by the method of claim 1.

16. A method of treating an aneuploid cancer in a subject in need thereof, comprising administering to the subject a therapeutic agent in an amount effective to reduce or prevent one or more symptoms of the aneuploid cancer in the subject,
wherein the therapeutic agent reduces the viability of aneuploid cancer cells more than the viability of isogenic control cells created from the aneuploid cancer cells according to the method of claim 1.

17. A method of treating a disease or disorder associated with an aneuploidy in a subject in need thereof, comprising
(i) screening one or more candidate active agents for selective toxicity against an aneuploid cell or aneuploid tissue obtained from the subject,
wherein the candidate active agents(s) is selected as being selectively toxic to the aneuploid cell or aneuploid tissue when the agent reduces viability or reproduction of the aneuploid cell or aneuploid tissue to a greater extent than a viable isogenic diploid-like cell or tissue or organoid prepared according to the method of claim 1, and
(ii) administering the selected candidate active agent(s) to the subject in an amount effective to treat the disease or disorder associated with an aneuploidy in the subject.

18. A method for screening of one or more candidate active agent(s) for selective cytotoxicity to aneuploid cancer cells or aneuploid cancer tissue, comprising:
(a) contacting a viable aneuploid cancer cell or aneuploid cancer tissue with one or more active agents under conditions suitable for cell or tissue culture,
wherein the aneuploid cancer cell or aneuploid cancer tissue comprises an additional chromosome or chromosome arm relative to an isogenic control cell or tissue, and
(b) selecting the one or more candidate active agent(s) as selectively toxic to the aneuploid cancer cell or aneuploid cancer tissue when the one or more candidate active agent(s) reduces the viability of the aneuploid cancer cell or aneuploid cancer tissue to a greater extent than the reduction in viability of the isogenic control cell or tissue contacted by the same candidate active agent(s) under the same conditions.

19. A mutant cell, wherein the mutant cells is
(i) a mutant A2058 cell comprising loss of a trisomic chromosome relative to an A2058 parental cell, optionally loss of 1q trisomy, 7p trisomy, 8q trisomy, or a combination thereof;
(ii) a mutant A2780 cell comprising loss of a trisomic chromosome relative to an A2780 parental cell, optionally loss of 1q trisomy;
(iii) a mutant AGS cell comprising loss of a trisomic chromosome relative to an AGS parental cell, optionally loss of 1q trisomy;
(iv) a mutant MCF10A cell comprising loss of a trisomic chromosome relative to an MCF10A parental cell, optionally loss of 1q trisomy; or
(v) a mutant HCT116 cell comprising loss of a trisomic chromosome relative to an HCT116 parental cell, optionally loss of 8q trisomy.

20. The cell of claim 19, selected from

| Parental cell line | Clone name | Method |
|---|---|---|
| A2058 | 1q-loss c1 | ReDACT-CO |
| A2058 | 1q-loss c2 | ReDACT-CO |
| A2058 | 1q-loss c3 | ReDACT-CO |
| A2058 | 1q-loss c4 | ReDACT-CO |
| A2780 | 1q-loss c1 | ReDACT-NS |
| A2780 | 1q-loss c2 | ReDACT-NS |
| A2780 | 1q-loss c3 | ReDACT-NS |
| A2780 | 1q-loss c4 | ReDACT-NS |
| A2780 | 1q-loss c5 | ReDACT-NS |
| A2780 | 1q-loss c6 | ReDACT-NS |
| A2780 | 1q-loss c8 | ReDACT-NS |
| AGS | 1q-loss c1 | ReDACT-NS |
| AGS | 1q-loss c4 | ReDACT-NS |
| AGS | 1q-loss c5 | ReDACT-NS |
| AGS | 1q-loss c6 | ReDACT-NS |
| AGS | 1q-loss c7 | ReDACT-NS |
| AGS | 1q-loss c8 | ReDACT-NS |
| AGS | 1q-loss c11 | ReDACT-TR |
| AGS | 1q-loss c12 | ReDACT-TR |
| MCF10A | 1q-loss c1 | ReDACT-TR |
| MCF10A | 1q-loss c2 | ReDACT-TR |
| MCF10A | 1q-loss c3 | ReDACT-TR |
| MCF10A | 1q-loss c4 | ReDACT-CO |
| A2058 | 7p-loss c1 | ReDACT-CO |
| A2058 | 7p-loss c2 | ReDACT-CO |
| A2058 | 7p-loss c3 | ReDACT-CO |
| A2058 | 8q-loss c1 | ReDACT-CO |
| A2058 | 8q-loss c2 | ReDACT-CO |
| HCT116 | 8q-loss c1 | ReDACT-CO |
| HCT116 | 8q-loss c2 | ReDACT-CO. |


SEQUENCE LISTING (48 sequences)

SEQ ID NO: 1 - DNA, 20 bp, synthetic construct
  source 1..20: mol_type = other DNA
gacattgattattgactagt

SEQ ID NO: 2 - DNA, 20 bp, synthetic construct
  source 1..20: mol_type = other DNA
ccatagagcccaccgcatcc

SEQ ID NO: 3 - DNA, 20 bp, synthetic construct
  source 1..20: mol_type = other DNA
actgttgacggcggcgatgt

SEQ ID NO: 4 - DNA, 18 bp, synthetic construct
  source 1..18: mol_type = other DNA
agaacagttgaagtcctt

SEQ ID NO: 5 - DNA, 19 bp, synthetic construct
  source 1..19: mol_type = other DNA
gtaacaacatgtagtaccg

SEQ ID NO: 6 - DNA, 19 bp, synthetic construct
  source 1..19: mol_type = other DNA
caggtgggttacaaaagac

SEQ ID NO: 7 - DNA, 20 bp, synthetic construct
  source 1..20: mol_type = other DNA
agtaaactccaacagacctg

SEQ ID NO: 8 - DNA, 20 bp, synthetic construct
  source 1..20: mol_type = other DNA
tgattcaattggaagccaat

SEQ ID NO: 9 - DNA, 19 bp, synthetic construct
  source 1..19: mol_type = other DNA
caggtgggttacaaaagac

SEQ ID NO: 10 - DNA, 20 bp, synthetic construct
  source 1..20: mol_type = other DNA
cagcttctgaggctctaccg

SEQ ID NO: 11 - DNA, 20 bp, synthetic construct
  source 1..20: mol_type = other DNA
ctaggttacagatagtctcg

SEQ ID NO: 12 - DNA, 20 bp, synthetic construct
  source 1..20: mol_type = other DNA
tcaatggtaaaatagatcga

SEQ ID NO: 13 - DNA, 20 bp, synthetic construct
  source 1..20: mol_type = other DNA
agctatcgggttgcactgtg

SEQ ID NO: 14 - DNA, 20 bp, synthetic construct
  source 1..20: mol_type = other DNA
caaatgacccacttgtccga

SEQ ID NO: 15 - DNA, 20 bp, synthetic construct
  source 1..20: mol_type = other DNA
tgttcaatggagatgtcccg

SEQ ID NO: 16 - DNA, 20 bp, synthetic construct
  source 1..20: mol_type = other DNA
gtaggtatagcagaccactg

SEQ ID NO: 17 - DNA, 20 bp, synthetic construct
  source 1..20: mol_type = other DNA
tgccccacattgtaccacag

SEQ ID NO: 18 - DNA, 20 bp, synthetic construct
  source 1..20: mol_type = other DNA
gattttggtagtcatgacga

SEQ ID NO: 19 - DNA, 20 bp, synthetic construct
  source 1..20: mol_type = other DNA
attatacccagccattcccg

SEQ ID NO: 20 - DNA, 20 bp, synthetic construct
  source 1..20: mol_type = other DNA
gcggaatcctaatgaccagg

SEQ ID NO: 21 - DNA, 20 bp, synthetic construct
  source 1..20: mol_type = other DNA
atgatgccacaatcacgtga

SEQ ID NO: 22 - DNA, 20 bp, synthetic construct
  source 1..20: mol_type = other DNA
acagcaagttgtctaacccg

SEQ ID NO: 23 - DNA, 19 bp, synthetic construct
  source 1..19: mol_type = other DNA
agatggccatggcgcggac

SEQ ID NO: 24 - DNA, 19 bp, synthetic construct
  source 1..19: mol_type = other DNA
gcatgggcggcatgaaccg

SEQ ID NO: 25 - DNA, 20 bp, synthetic construct
  source 1..20: mol_type = other DNA
actgttgacggcggcgatgt

SEQ ID NO: 26 - DNA, 20 bp, synthetic construct
  source 1..20: mol_type = other DNA
gctgataccgtcggcgttgg

SEQ ID NO: 27 - DNA, 20 bp, synthetic construct
  source 1..20: mol_type = other DNA
ctcccccgccgagcccgggt

SEQ ID NO: 28 - DNA, 20 bp, synthetic construct
  source 1..20: mol_type = other DNA
caaaatgctgccgaggccct

SEQ ID NO: 29 - DNA, 20 bp, synthetic construct
  source 1..20: mol_type = other DNA
ggttaggaaggacggagaag

SEQ ID NO: 30 - DNA, 20 bp, synthetic construct
  source 1..20: mol_type = other DNA
ttaacaggccgtggttagga

SEQ ID NO: 31 - DNA, 200 bp, synthetic construct
  source 1..200: mol_type = other DNA
tttcatagacgttcttccttaagtcagcttgaataagtagtaggaggaggctcaggaaaacatttattaatgttttcataaccaaatgtgacatagtcatctttgcgaggtacacaattgagccaagctctaagagcctatccctgtatctaggttttgtccttacatgtatataacctagacattgattattgactagt

SEQ ID NO: 32 - DNA, 200 bp, synthetic construct
  source 1..200: mol_type = other DNA
tcaccatagctatcctctgaccatcacctgcctggacaatatatctgtctcttatggaaaagaaaggcaagaactgggaaggaacaaattcaagtgggcttttagcatagtaagagaacactgtccttcagtctgatcatttcagggtggcagggtatttttgtactactggagaggaaaccatagagcccaccgcatcc

SEQ ID NO: 33 - DNA, 23 bp, synthetic construct
  source 1..23: mol_type = other DNA
tgattgtcgaagaaccatttcgg

SEQ ID NO: 34 - DNA, 23 bp, synthetic construct
  source 1..23: mol_type = other DNA
tgcagggatcaaaaagtttggag

SEQ ID NO: 35 - DNA, 22 bp, synthetic construct
  source 1..22: mol_type = other DNA
ttttgatccctgcaactcagtg

SEQ ID NO: 36 - DNA, 22 bp, synthetic construct
  source 1..22: mol_type = other DNA
gtctcgtggtcttttctcacat

SEQ ID NO: 37 - DNA, 20 bp, synthetic construct
  source 1..20: mol_type = other DNA
tgcaccaccaactgcttagc

SEQ ID NO: 38 - DNA, 21 bp, synthetic construct
  source 1..21: mol_type = other DNA
ggcatggactgtggtcatgag

SEQ ID NO: 39 - DNA, 28 bp, synthetic construct
  source 1..28: mol_type = other DNA
gagctagctgctgttttctaatgtttgc

SEQ ID NO: 40 - DNA, 24 bp, synthetic construct
  source 1..24: mol_type = other DNA
cataaaaggaaactttcggagcgc

SEQ ID NO: 41 - DNA, 21 bp, synthetic construct
  source 1..21: mol_type = other DNA
tcagttagcctcccccatctc

SEQ ID NO: 42 - DNA, 23 bp, synthetic construct
  source 1..23: mol_type = other DNA
gtaatgacaagcgcccagataac

SEQ ID NO: 43 - DNA, 21 bp, synthetic construct
  source 1..21: mol_type = other DNA
ggactctcagcaattaaactg

SEQ ID NO: 44 - DNA, 20 bp, synthetic construct
  source 1..20: mol_type = other DNA
cctgtattctccactctacc

SEQ ID NO: 45 - DNA, 24 bp, synthetic construct
  source 1..24: mol_type = other DNA
cgtcgatggaggagtttgtaaatg

SEQ ID NO: 46 - DNA, 22 bp, synthetic construct
  source 1..22: mol_type = other DNA
gcacagagagtgaacatcatgg

SEQ ID NO: 47 - DNA, 23 bp, synthetic construct
  source 1..23: mol_type = other DNA
cgatacacgtctgcagtcaactg

SEQ ID NO: 48 - DNA, 29 bp, synthetic construct
  source 1..29: mol_type = other DNA
gacatactcccaaggaaagtaaagttccc